(19) United States
(12) Patent Application Publication
(10) Pub. No.: US 2024/0052012 A1
(43) Pub. Date: Feb. 15, 2024

(54) PLATELET ALPHA-GRANULES FOR DELIVERY OF MULTIPLE PROTEINS

(71) Applicant: CSTS Health Care Inc., Toronto (CA)

(72) Inventors: Giannoula Lakka KLEMENT, Toronto (CA); Qian LIU, Toronto (CA)

(21) Appl. No.: 18/356,688

(22) Filed: Jul. 21, 2023

Related U.S. Application Data
(63) Continuation of application No. PCT/US2022/014107, filed on Jan. 27, 2022.
(60) Provisional application No. 63/142,402, filed on Jan. 27, 2021.

(51) Int. Cl. C07K 14/705; C07K 14/755; A61K 47/69; A61P 35/00

(52) U.S. Cl. CPC C07K 14/70564; C07K 14/755; A61K 47/6901; A61P 35/00

(57) ABSTRACT

The present disclosure provides compositions and methods comprising platelets loaded with at least two agents, with each agent being loaded into a distinct α-granule type of the platelet. Agents loaded into platelets are generally protected from degradation and the subject is protected from toxicity, if any, from the agent. These benefits, coupled with the platelets' natural ability to home to sites of injury, inflammation, and/or angiogenesis, helps ensure that a therapeutically effective amounts of the agents are delivered to a target site.

### CROSS-REFERENCE TO RELATED APPLICATIONS

This application is a Continuation of International Application No. PCT/US2022/014107 filed Jan. 27, 2022, which claims priority to U.S. 63/142,402, filed Jan. 27, 2021, the contents of which is incorporated by reference in its entirety.

### SEQUENCE LISTING

The instant application contains a Sequence Listing which has been submitted electronically in XML format and is hereby incorporated by reference in its entirety. Said XML copy, created on June 27, 2023, is named 58533702301.xml and is 18,238 bytes in size.

### BACKGROUND

Therapeutic compounds that are systemically administered can degrade prior to arrival to their target site; thus, if they arrive at all, their dose may be too low to achieve a therapeutic effect. Platelets naturally home to sites of injury, inflammation, and/or angiogenesis and are known to transport native cargos to these sites. If exogenous therapeutic agents could be loaded into platelets, the agents should be protected from the degradation that would occur following the agent's systemic administration. However, no mechanisms for loading exogenous, therapeutic agents into platelet's alpha granules has been described. Thus, there is an unmet need for loaded platelets that can deliver exogenous therapeutic agents to sites of injury, inflammation, and/or angiogenesis.

### SUMMARY

An aspect of the present disclosure is a composition, e.g., for treating a disease or disorder. The composition comprises a first compound comprising a first agent and a first polypeptide, wherein the first polypeptide comprises a first glycosaminoglycan (GAG)-binding peptide which is capable of binding a GAG in a first alpha granule type of a platelet; and a second compound comprising a second agent and a second polypeptide, wherein the second polypeptide comprises a second glycosaminoglycan (GAG)-binding peptide which is capable of binding a GAG in a second alpha granule type of the platelet.

In embodiments, the first GAG-binding peptide preferentially binds to chondroitin sulfate (CS) and the second GAG-binding peptide preferentially binds to heparan sulfate (HS).

In various embodiments, the first GAG-binding peptide preferentially binds to chondroitin sulfate A (CSA) and does not preferably bind to heparan sulfate (HS).

In some embodiments, the first alpha granule type is a P-selectin associated granule and the second alpha granule type von Willebrand factor (VWF) associated granule.

In embodiments, the contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin derivative such as tissue plasminogen activator (tPA).

In various embodiments, the contents of the first alpha granule type are released at a lower concentration of thrombin than the concentration of thrombin needed to provide release of the contents of the second alpha granule type.

In some embodiments, the contents first alpha granule type is released before the contents of the second alpha granule type are released.

In embodiments, the first and the second GAG-binding peptides are each between about 8 amino acids and about 14 amino acids in length.

In various embodiments, one or both of the first and the second GAG-binding peptides comprises at least one charged amino acid. In some cases, both of the first and the second GAG-binding peptides comprise at least one charged amino acid.

In some embodiments, one or both of the first and the second GAG-binding peptides comprises at least one proline, arginine, and/or isoleucine. In some cases, both of the first and the second GAG-binding peptides comprise at least at least one proline, arginine, and/or isoleucine.

In embodiments, the first and the second GAG-binding peptides independently comprise an amino acid sequence that is at least about 70% identical to one of SEQ ID NO: 1 to SEQ ID NO: 13.

In various embodiments, the first and the second GAG-binding peptides independently comprise an amino acid sequence that is at least about 80% identical to one of SEQ ID NO: 1 to SEQ ID NO: 13.

In some embodiments, the first and the second GAG-binding peptides independently comprise an amino acid sequence that is at least about 90% identical to one of SEQ ID NO: 1 to SEQ ID NO: 13.

In embodiments, the first and the second GAG-binding peptides independently comprise a charged amino acid at position 1, position 4, position 7, or position 9 with respect to any one of SEQ ID NO: 1 to SEQ ID NO: 13.

In various embodiments, the first and the second GAG-binding peptides independently comprise a proline, arginine, and/or isoleucine at position 1, position 4, position 7, and/or position 9 with respect to any one of SEQ ID NO: 1 to SEQ ID NO: 13.

In some embodiments, the first and the second GAG-binding peptides independently comprise at least amino acids.

In embodiments, the first and/or the second GAG-binding peptides independently comprise 11 amino acids.

In various embodiments, the first and the second GAG-binding peptides independently consist of 11 amino acids.

In some embodiments, the first and the second GAG-binding peptides independently comprise the amino acid sequence of one of SEQ ID NO: 1 to SEQ ID NO: 13.

In embodiments, the first GAG-binding peptide comprises an amino acid sequence that is at least about 90% identical to SEQ ID NO: 1 and the second GAG-binding peptide comprises an amino acid sequence that is at least about 90% identical to SEQ ID NO: 2. In some cases the first GAG-binding peptide comprises the amino acid sequence of SEQ ID NO: 1 and the second GAG-binding peptide comprises the amino acid sequence of SEQ ID NO: 2. The first GAG-binding peptide may consist of the amino acid sequence of SEQ ID NO: 1 and the second GAG-binding peptide may consist of the amino acid sequence of SEQ ID NO: 2.

In various embodiments, the first polypeptide consists of the first GAG-binding peptide and the second polypeptide consists of the second GAG-binding peptide.

In some embodiments, the N-terminal of the first polypeptide is directly or indirectly linked to the first agent and/or the N-terminal of the second polypeptide is directly or indirectly linked to second first agent.

In embodiments, the C-terminal of the first polypeptide is directly or indirectly linked to the first agent and/or the C-terminal of the second polypeptide is directly or indirectly linked to second first agent.

In various embodiments, the first agent is indirectly linked to the first polypeptide via a first linker and/or the second agent is indirectly linked to the second polypeptide via a second linker. In some cases, the first linker and/or the second each comprise one or more atoms. The first linker and/or the second may each comprise a polymer of repeating units. The first linker and/or the second linker may each comprise a chain of amino acids.

In some embodiments, the first agent is directly linked to the first polypeptide and/or the second agent is directly linked to the second polypeptide.

In embodiments, the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

In various embodiments, the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet. In some cases, the first agent and/or the second agent comprises an antibody or a fluorescent moiety.

In some embodiments, the first agent and/or the second agent is harmful to mammalian cells and/or is toxic to a subject and/or the first agent and/or the second agent is susceptible to degradation when administered directly into the bloodstream of a subject.

In embodiments, the first compound and/or the second compound further comprises a fluorescent moiety.

In various embodiments, the first GAG-binding peptide and/or the second GAG-binding peptide also preferentially binds serglycin, perlecan, dermatan sulfate, keratan sulfate, and/or GPIIb/IIIa.

In some embodiments, the composition further comprises a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

Another aspect of the present disclosure is an isolated platelet. The isolated platelet comprises at least one copy of a first compound comprising a first agent and a first polypeptide, wherein the first polypeptide comprises a first glycosaminoglycan (GAG)-binding peptide which is capable of binding a GAG in a first alpha granule type of a platelet; and at least one copy of a second compound comprising a second agent and a second polypeptide, wherein the second polypeptide comprises a second glycosaminoglycan (GAG)-binding peptide which is capable of binding a GAG in a second alpha granule type of the platelet.

In embodiments, the platelet is a synthetic, an allogeneic, an autologous, or a modified heterologous platelet.

In various embodiments, the platelet is an autologous platelet.

In some embodiments, the platelet is an allogeneic platelet.

In embodiments, the platelet is obtained from platelet rich plasma.

In various embodiments, the platelet comprises 1 to 1000 copies of the first compound and 1 to 1000 copies of the second compound. In some cases, the 1 to 1000 copies of the first compound are loaded into a first alpha granule type of a platelet and the 1 to 1000 copies of the second compound are loaded into a second alpha granule type of the platelet. The least one copy of the first compound may be loaded into a second alpha granule type of a platelet and at least one copy of the second compound may be loaded into a first alpha granule type of the platelet.

In some embodiments, the first GAG-binding peptide preferentially binds to chondroitin sulfate (CS) and the second GAG-binding peptide preferentially binds to heparan sulfate (HS).

In embodiments, the first GAG-binding peptide preferentially binds to chondroitin sulfate A (CSA) In various embodiments, the first alpha granule type is a P-selectin associated granule and the second alpha granule type von Willebrand factor (VWF) associated granule.

In some embodiments, the contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin such as tissue plasminogen activator (tPA).

In embodiments, the contents of the first alpha granule type are released at a lower concentration of thrombin than the concentration of thrombin needed to provide release of the contents of the second alpha granule type.

In various embodiments, the contents first alpha granule type is released before the contents of the second alpha granule type are released.

In some embodiments, the first and the second GAG-binding peptides are each between about 8 amino acids and about 14 amino acids in length. In some cases, or both of the first and the second GAG-binding peptides comprises at least one charged amino acid. Both the first and the second GAG-binding peptides may comprise at least one charged amino acid.

In embodiments, one or both of the first and the second GAG-binding peptides comprises at least one proline, arginine, and/or isoleucine. In some cases, both of the first and the second GAG-binding peptides comprise at least at least one proline, arginine, and/or isoleucine.

In various embodiments, the first and the second GAG-binding peptides independently comprise an amino acid sequence that is at least about 70% identical to one of SEQ ID NO: 1 to SEQ ID NO: 13.

In some embodiments, the first and the second GAG-binding peptides independently comprise an amino acid sequence that is at least about 80% identical to one of SEQ ID NO: 1 to SEQ ID NO: 13.

In embodiments, the first and the second GAG-binding peptides independently comprise an amino acid sequence that is at least about 90% identical to one of SEQ ID NO: 1 to SEQ ID NO: 13.

In various embodiments, the first and the second GAG-binding peptides independently comprise a charged amino acid at position 1, position 4, position 7, or position 9 with respect to any one of SEQ ID NO: 1 to SEQ ID NO: 13.

In some embodiments, the first and the second GAG-binding peptides independently comprise a proline, arginine, and/or isoleucine at position 1, position 4, position 7, and/or position 9 with respect to any one of SEQ ID NO: 1 to SEQ ID NO: 13.

In embodiments, the first and the second GAG-binding peptides independently comprise at least 10 amino acids.

In various embodiments, the first and the second GAG-binding peptides independently comprise 11 amino acids.

In some embodiments, the first and the second GAG-binding peptides independently consist of 11 amino acids.

In embodiments, the GAG-binding peptide consists of the amino acid sequence of one of SEQ ID NO: 1 to SEQ ID NO: 13.

In various embodiments, the first GAG-binding peptide comprises an amino acid sequence that is at least about 90% identical to SEQ ID NO: 1 and the second GAG-binding peptide comprises an amino acid sequence that is at least about 90% identical to SEQ ID NO: 2.

In some embodiments, the first GAG-binding peptide comprises the amino acid sequence of SEQ ID NO: 1 and the second GAG-binding peptide comprises the amino acid sequence of SEQ ID NO: 2.

In embodiments, the first GAG-binding peptide consists of the amino acid sequence of SEQ ID NO: 1 and the second GAG-binding peptide consists of the amino acid sequence of SEQ ID NO: 2.

In various embodiments, the first polypeptide consists of the first GAG-binding peptide and the second polypeptide consists of the second GAG-binding peptide.

In some embodiments, the N-terminal of the first polypeptide is directly or indirectly linked to the first agent and/or the N-terminal of the second polypeptide is directly or indirectly linked to second first agent.

In embodiments, the C-terminal of the first polypeptide is directly or indirectly linked to the first agent and/or the C-terminal of the second polypeptide is directly or indirectly linked to second first agent.

In various embodiments, the first agent is indirectly linked to the first polypeptide via a first linker and/or wherein the second agent is indirectly linked to the second polypeptide via a second linker. In some cases, the first linker and/or the second each comprise one or more atoms. The first linker and/or the second may each comprise a polymer of repeating units. The first linker and/or the second may each comprise a chain of amino acids.

In some embodiments, the first agent is directly linked to the first polypeptide and/or the second agent is directly linked to the second polypeptide.

In embodiments, the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

In various embodiments, the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet. In some cases, the first agent and/or the second agent comprises an antibody and/or comprises a fluorescent moiety.

In some embodiments, the first agent and/or the second agent is harmful to mammalian cells and/or is toxic to a subject and/or the first agent and/or the second agent is susceptible to degradation when administered directly into the bloodstream of a subject.

In embodiments, the first compound and/or the second compound further comprises a fluorescent moiety.

In various embodiments, the first GAG-binding peptide and/or the second GAG-binding peptide also preferentially binds serglycin, perlecan, dermatan sulfate, keratan sulfate, and/or GPIIb/IIIa.

In some embodiments, the isolated platelet further comprises at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

Yet another aspect of the present disclosure is a pharmaceutical composition comprising an isolated platelet of any herein disclosed aspect or embodiment and one or more pharmaceutically acceptable excipients.

In embodiments, the pharmaceutical composition further comprises a second isolated platelet comprising at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

In various embodiments, the pharmaceutical composition further comprises a second isolated platelet comprising at least one copy of the first compound or further comprising a third isolated platelet comprising at least one copy of a second compound.

In some embodiments, the pharmaceutical composition further comprises a second isolated platelet comprising at least one copy of the first compound and comprising a third isolated platelet comprising at least one copy of a second compound.

In an aspect, the present disclosure provides a use of any herein disclosed pharmaceutical composition for treating a disease or a disorder. In embodiments, the disease or disorder is a cancer.

In another aspect, the present disclosure provides a use of any herein disclosed isolated platelet of any or any herein disclosed pharmaceutical composition in the manufacture of a medicament for treating a disease or disorder. In various embodiments, the disease or disorder is a cancer.

In yet another aspect, the present disclosure provides a method for treating a disease or disorder in a subject in need thereof. The method comprises a step of administering to the subject a therapeutically effective amount of any herein disclosed pharmaceutical composition.

An aspect of the present disclosure is a method for treating a disease or disorder in a subject in need thereof. The method comprising a step of administering to the subject a therapeutically effective amount of any herein disclosed composition.

In some embodiments of the above methods, the contents of the first alpha granule type is released at a target site before the contents of second alpha granule type is released.

In embodiments of the above methods, the method further comprises a step of administering to the subject a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase. In some cases, the second pharmaceutical composition promotes release of a first compound from a first alpha granule type and the third pharmaceutical composition promotes release of a second compound from a second alpha granule type. The second pharmaceutical composition and/or the third pharmaceutical composition may be administered after the pharmaceutical composition is administered. The pharmaceutical composition may be administered at least twice before the second pharmaceutical composition and/or the third pharmaceutical composition is administered.

In various embodiments of the above methods, the disease or disorder is a cancer.

In some embodiments of the above methods, the disease of disorder is inflammation.

In various embodiments of the above methods, the disease of disorder is a side effect of an implant, graft, stent, or prosthesis.

In embodiments of the above methods, the disease of disorder is caused by a defective gene.

In some embodiments of the above methods, the disease of disorder is an injury.

Another aspect of the present disclosure is a method for manufacturing a loaded platelet. The method comprising steps of: obtaining a platelet; contacting the platelet in vitro or ex vivo with any herein disclosed composition; and allowing contact between the platelet and the composition to progress until the first compound is internalized by a first alpha granule type of the platelet and the second compound is internalized by a second alpha granule type of the platelet, thereby producing a loaded platelet.

Yet another aspect of the present disclosure in a method for manufacturing a loaded platelet. The method comprising steps of: obtaining a platelet; contacting the platelet in vitro or ex vivo with a first compound comprising a first agent and a first polypeptide, wherein the first polypeptide comprises a first glycosaminoglycan (GAG)-binding peptide which is capable of binding a GAG in a first alpha granule type of a platelet; and contacting the platelet in vitro or ex vivo with a second compound comprising a second agent and a second polypeptide, wherein the second polypeptide comprises a second glycosaminoglycan (GAG)-binding peptide which is capable of binding a GAG in a second alpha granule type of the platelet.

In embodiments, the contacting the platelet with the first compound and the contacting the platelet with the second compound are contemporaneous.

In various embodiments, the contacting the platelet with the first compound and the contacting the platelet with the second compound are sequential.

In some embodiments, the method further comprises contacting the platelet in vitro or ex vivo with a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

In an aspect, the present disclosure provides a kit for treating a disease or disorder. The kit comprising any herein disclosed isolated platelet and instructions for use.

In another aspect, the present disclosure provides a kit for treating a disease or disorder. The kit comprising any herein disclosed pharmaceutical composition and instructions for use.

In embodiments, the kit further comprises a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase.

In yet another aspect, the present disclosure provides a kit for manufacturing a loaded platelet. The kit comprising any herein disclosed composition and instructions for use.

Any aspect or embodiment disclosed herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings (also “figure” and “FIG.” herein), of which:

FIG. 1A and FIG. 1B are graphs showing the ability of illustrative glycosaminoglycan (GAG)-binding peptides to sequester attached cargos into platelets.

FIG. 2A are immunofluorescent images and FIG. 2B is a graph demonstrating the ability of illustrative glycosaminoglycan (GAG)-binding peptides to sequester attached cargos into alpha granules of platelets.

FIG. 3A is a schematic depicting isothermal titration calorimetry (ITC) experiments. Graphical representations of ITC dissociation kinetics for chondroitin sulfate A (CSA) titrated into cells withholding illustrative GAG-binding peptides are shown in FIG. 3B (for the GAG-binding peptide of SEQ ID NO: 1), FIG. 3C (for the GAG-binding peptide of SEQ ID NO: 2), and FIG. 3D for a charge-free ligand. The data of FIG. 3B is tabulated in FIG. 3E and the data of FIG. 3C is tabulated in FIG. 3F.

FIG. 4A shows the peptide amino acid sequences including the control peptide (CFL; EGGIWFPYGGF; SEQ ID NO: 14) and the two PALs (PAL1; ERRIWFPYRRF; SEQ ID NO: 1 and PAL2; RFRWPYRIREF; SEQ ID NO: 2). FIG. 4B and FIG. 4C shows affinity chromatography data for the three illustrative GAG-binding peptides of the previous figures albeit when binding to heparan sulfate (HS). FIG. 4D shows a conjugation diagram for Fam-PAL-Lucitanib. FIG. 4E shows % FGFR activity for various conjugates. Conjugated Lucitanib keeps FGFR1 inhibiting function as tested using ADP-Glo kit from Promega. The slight drop of EC50 may be due to the decreased solubility of conjugates. The Abbreviations included in this figure and elsewhere, where appropriate are as follows: A-PAL1—Alexa647-labelled-PAL1; A-PAL2—Alexa647-labelled-PAL2; Fam-PAL1—Fam-labelled-PAL1; Fam-PAL2—Fam-labelled-PAL2; Fam-PAL1-Luci—Fam-PAL1-conjugated Lucitanib; Fam-PAL2-Luci—Fam-PAL2-conjugated Lucitanib; Fam-CFL-Luci—Fam-CFL-conjugated Lucitanib; and Fam-L-Luci—Fam-linker-conjugated Lucitanib.

FIG. 5 is a graph demonstrating loading of an illustrative compound comprising a glycosaminoglycan (GAG)-binding peptide and an agent into platelets.

FIG. 6A are immunofluorescent images and FIG. 6B is a graph demonstrating the ability of illustrative compounds comprising a glycosaminoglycan (GAG)-binding peptide and an agent to load into alpha granules of platelets.

FIG. 7A to FIG. 7C include graphical representations of ITC dissociation kinetics for chondroitin sulfate A (CSA) titrated into cells withholding the illustrative compound comprising PAL1 (FIG. 7A), the illustrative compound comprising PAL2 (FIG. 7B), and the control compound comprising CFL (FIG. 7C). The data of FIG. 7A is tabulated in FIG. 7D, the data of FIG. 7B is tabulated in FIG. 7E, and the data of FIG. 7C is tabulated in FIG. 7F.

FIG. 8 shows affinity chromatography data for the three illustrative compounds of the previous figures albeit when binding to heparan sulfate (HS).

FIG. 9A include graphical representations of ITC dissociation kinetics for chondroitin sulfate A (CSA) titrated into cells withholding the additional illustrative compounds. These additional illustrative compounds are identified as PAL1A to PAL11A and, respectively, comprise GAG-binding peptides having amino acid sequences of SEQ ID NO: 3 to SEQ ID NO: 13. The data of FIG. 9A is tabulated in FIG. 9B to FIG. 9L. FIG. 9M is a graph depicting the average dissociation constant for the additional illustrative compounds and a negative control compound.

FIG. 10A is a diagram showing illustrative steps in conjugating a GAG-binding peptide to an agent when forming a compound of the present disclosure. FIG. 10B are immunofluorescent images and FIG. 10C is a graph demonstrating the ability of illustrative compounds comprising a glycosaminoglycan (GAG)-binding peptide and an agent to load into alpha granules of platelets.

FIG. 11 are diagrams showing that platelet levels of bFGF, VEGF, PDGF, and endostatin change just prior to tumor escape from dormancy with the balance being towards stimulators of tumor growth.

FIG. 12 are MS expression maps showing that platelets actively sequester cancer specific proteins, and do not actively sequester non-specific proteins such as albumin

FIG. 13 is a table showing that platelets contain both stimulators (VEGF, bFGF, PDGF) and inhibitors (PF4, endostatin) of angiogenesis.

FIG. 14 show SELDI-ToF analyses of platelets from subjects with localized prostate cancer undergoing positive lifestyle interventions (blue) and those undergoing watchful waiting without change in lifestyle at 6 months post intervention.

FIG. 15A and FIG. 15B are graphs showing inhibition of the respective receptor does not inhibit platelet sequestration (FIG. 15A), but inhibition of heparin binding by surfen results in significant inhibition of protein sequestration by platelet a granules (FIG. 15B).

FIG. 16 are immunofluorescent images showing that VEGF and endostatin inhabit separate platelet α-granules.

FIG. 17 are immunofluorescent images showing that a stimulator of angiogenesis (e.g., VEGF) localizes with P-selectin in α-granules.

FIG. 18 are immunofluorescent images showing that endostatin is in a separate and distinct α-granule compartment and co-localizes with von Willebrand factor (VWF) rather than with P-selectin

FIG. 19 include schematics summarizing the sequential release of proteins in wounds healing and local concentration gradients of proteinase activated receptor 1 (PAR1) and PAR4.

FIG. 20 is a graph showing sequestration of growth factors by GAGs on the surface of the hemangioendothelioma cells (EOMA), confirming the sequestration is heparin dependent and increases with thrombin presence.

FIG. 21 is a graph showing proliferation of murine hemangioendothelioma cells (EOMA) in response to growth factors released from platelet formed provisional matrix.

FIG. 22 are immunofluorescent images showing that platelets form a provisional matrix that can exchange proteins with endothelial cells upon tumor activation.

FIG. 23A are immunofluorescent images showing that PAL1 or PAL2 conjugates load into platelets. FIG. 23B are graphs showing dose responsive loading of Fam-PAL1 or Fam-PAL2 (top) and Fam-PAL1-Lucitanib or Fam-PAL2-Lucitanib (bottom) into platelets. The loaded platelets appear to have retained the morphology of a resting, fully functional platelet.

FIG. 24 are immunofluorescent images showing that PAL1 and PAL2 have different subcellular localizations, i.e., have a preference for distinct alpha granules.

FIG. 25A and FIG. 25B are structural diagrams showing that PAL1 (violet colored in the figures) and PAL2 (pale blue in the figures) may bind Chondroitin Sulphate A (CSA; FIG. 25A) and Heparan Sulphate (HS; FIG. 25B) differently.

FIG. 26A and FIG. 26B show that when PAL1 is conjugated onto a small molecule, the PAL1 can guide the respective molecule into a platelet α-granule.

FIG. 27A is a flow chart illustrating steps in fractionating platelet granules. FIG. 27B are images showing sucrose gradients (top is a cartoon) and (bottom is a photograph) for separating platelet granules. FIG. 27C are western blots of granule fractions. FIG. 27D includes immunofluorescent images of the granule fractions and which are labeled for FAM, PF4, MRP4, or VEGF. FIG. 27E are graphs quantifying the markers PF4, VEGF, and MRP4 in each of the granule fractions. FIG. 27F are graphs showing localization of PAL conjugates in certain granule fractions. FIG. 27G show Pearson Correlation Analysis (PCA) of the images shown in FIG. 27E.

### DETAILED DESCRIPTION

The present disclosure relates to isolated platelets loaded with more than one compounds. A first compound comprises a first agent and a first polypeptide, with the first polypeptide comprising a first glycosaminoglycan (GAG)-binding peptide which is capable of binding a GAG in a first alpha granule type of a platelet. The second compound comprises a second agent and a second polypeptide, with the second polypeptide comprising a second GAG-binding peptide which is capable of binding a GAG in a second alpha granule type of the platelet. The first alpha granule type and the second alpha granule type are characterized by the predominance of different GAG.

Notably, the first GAG-binding peptide preferably binds to a GAG type that is predominantly found on a first alpha granule type (i.e., a P-selectin type granule) and the second GAG-binding peptide preferably binds to a GAG type that is predominantly found on a second alpha granule type (i.e., a vWF type granule). By engineering first compounds with a first GAG-binding peptide and second compounds with a second GAG-binding peptide, a platelet can be loaded with two active agent and into two distinct granule types. Of importance, each granule type has a separate release profile, based in part on the identity of the thrombin receptor (i.e., proteinase activated receptor 1 (PAR1) and PAR4) that is associated with the granule type. PAR1 is a high affinity thrombin receptor which is triggered first (in low thrombin conditions) and releases P-selectin type α-granules (here referred to as a first α-granule type) whereas PAR4 is the low affinity receptor which is triggered only when sufficient amounts of thrombin has accumulated to release the vWF α-granules (here referred to as a second α-granule type). Optionally, release of the contents of an alpha granule may be induced in response to contact with a matrix metalloproteinase (MMP), peroxidase, phosphohydrolase, plasmin, or a plasmin derivative such as tissue plasminogen activator (tPA), these release inducers may be administered to a subject in a pharmaceutical composition and at a time that facilitates release of the agents as needed to promote a therapeutic response.

A loaded platelet of the present disclosure is able to release its content in a temporally and spatially controlled manner, via the distinct α-granule types. In addition to the enzymatic activity of thrombin and its derivatives, other tissue resident proteases such as peroxidase, phosphohydrolase, plasmin, or a plasmin derivative such as tissue plasminogen activator (tPA) also selectively release platelet alpha granule contents in a temporally and spatially controlled manner.

Thus, a platelet is loaded, at least, with a first drug in a first α-granule type that has an early release profile and with a second drug in a second α-granule type that has a later release profile. Consequently, a therapeutic can be designed that releases a first agent that is needed during an early phase of treatment and that releases a second agent that is needed during a later phase of treatment. Additionally, the timing of release of the distinct α-granule types can be controlled by administration to a subject of a pharmaceutical composition that stimulates release (e.g., thrombin, metalloproteinase (MMP), peroxidase, phosphohydrolase, plasmin, or a plasmin derivative such as tissue plasminogen activator (tPA)).

The present invention is based, in part, on the creation of platelets loaded with a plurality agents and into distinct α-granule types. The loaded platelets that provide directed therapeutics to sites of injury, pathological inflammation, and/or angiogenesis. Such agents sequestered within platelets, e.g., platelet alpha granules, are generally protected from degradation, which may occur upon systemic administration. This benefit, coupled with platelets' natural ability to home to sites of injury, inflammation, and/or angiogenesis helps to ensure that a therapeutically effective amount of the agent is delivered to a target site. Additionally, since the platelets useful in the present invention are loaded with a plurality of different agents and into distinct α-granule types, the different agents are released from platelets in a spatially- and temporally controlled fashion. Accordingly, the present invention provides directed and controlled therapeutics to sites of injury (e.g., for treating chronic wounds), pathological inflammation (e.g., for treating injury to joints or lungs), and/or angiogenesis (e.g., for treating cancer).

Prior to the present invention, it was counterintuitive that agents could be internalized into platelets by being anchored to specific glycosaminoglycans (GAG) in alpha granules and that a specific GAG-binding peptide can be used to facilitate the process of internalization, let alone specifically loading distinct α-granule types with different compounds Indeed, previously, there was no known method for loading agents into platelet alpha granules and it was unknown that subpopulations of alpha granule types could be loaded with different agents, thereby allowing spatially- and/or temporally controlled release of the different agents. Such controlled release allows sequential delivery of different agents, which could result in a synergistic therapeutic effect that may not be observed when the different agents are administered simultaneously.

The present invention provides numerous benefits, including, but not limited to:
(1) Targeted delivery of an agent to the site of a primary tumor or metastatic growth, which avoids the need for systemic administration of high doses of the agent; thus, lower doses of the agent are needed to achieve therapeutically effective concentrations of the agent at the target site;
(2) Agents sequestered in platelet alpha granules are unable to bind off-target receptors; thus, side effects (e.g., toxicity) associated with systemic administration of the agent alone is avoided;
(3) Agents sequestered in platelet alpha granules are protected from degradation by natural processes (e.g., tissue proteases); thus, the agent's half-life is extended relative to the agent when systemically administered alone;
(4) Selective loading of agents into distinct α-granule types, each of which have separate release profiles, thereby allowing release of different agents from platelets in a spatially- and temporally controlled fashion; and
(5) Release of the contents of loaded platelets of an alpha granule may be induced in response to contact with a release inducer which may be administered to a subject in a pharmaceutical composition and at a time that facilitates release of the agents as needed to promote a therapeutic response.
   Notably, the loaded platelets of the present disclosure remain in a resting, fully functional platelet, rather than becoming activated by the loading process which would make the platelets pro-coagulant.

### Platelets, Platelet Granules, and Glycosaminoglycans

The present invention provides compounds, pharmaceutical compositions, and methods for treating diseases, disorders, or injuries in which platelets are naturally first responders and in which platelets ameliorate, at least, the initial symptoms of the disease, disorder, or injury. Illustrative diseases, disorders, or injuries include, but are not limited to, cancer, rheumatoid arthritis, diabetic retinopathy, obesity, atherosclerosis, ischemic heart and limb disease, ulcerative colitis, stroke, burns, and other wounds. Under physiological conditions, circulating platelets maintain the health and stability of tissues.

New information about the role of platelets in wound and tumor microenvironment has emerged; see, e.g., Klement et al., “Platelets actively sequester angiogenesis regulators”, Blood. 2009; 113: 2835-42 and Klement et al., “The Role of Platelets in Angiogenesis. In: Michelson A, editor. Platelets. Third ed. Philadelphia, PA: Mosby Elsevier; 2013. p. 487-503. However, an understanding of the complexity of platelet/tissue interaction and the role of platelets in modulating tissue growth and angiogenesis has been slow to emerge. It is known that platelets contain different types of granules, including alpha granules, dense granules, and lysosomes, which perform different functions. The alpha granules, which normally contain growth factors, are the most prevalent type of granule. See, Blair and Flaumenhaft, “Platelet alpha-granules: basic biology and clinical correlates”. Blood Reviews. 2009, 23 (4): 177-89 and Harrison and Cramer, “Platelet alpha-granules”. Blood Reviews. 1993, 7 (1): 52-62. Normally, an alpha granule's cargo predominantly comprises inhibitors of angiogenesis; see, e.g., Peterson et al., “Normal ranges of angiogenesis regulatory proteins in human plate-lets.” American journal of hematology. 2010; 85: 487-93. However, when a subject has cancer, platelet cargoes change and the alpha granules become predominantly loaded with stimulators; see, Peterson et al., American journal of hematology. 2010; 85: 487-93 and Peterson et al., “VEGF, PF4 and PDGF are elevated in platelets of colorectal cancer patients.” Angiogenesis. 2012; 15: 265-73.

The present invention is based, in part, on the discovery that cargo can be loaded in distinct alpha granule types and that this loading is not receptor mediated. Instead, cargo loading into platelets, and specifically into their alpha granules, relies on the binding to glycosaminoglycans (GAG) in the alpha granules of the platelets and that one type of alpha granule is characterized by one GAG and other markers and another type of alpha granule is characterized by a second GAG and other markers. When platelets are contacted with a non-specific GAG inhibitor (i.e., Surfen), reduced amounts of cargos are loaded into platelets.

The present invention is further based, in part, on the discovery that a platelet's cargo is organized by function, with stimulators and inhibitors of angiogenesis taken up into distinct subsets of platelet alpha granules; this distinction is based on the cargo's binding affinities to chondroitin sulfate or heparan sulfate, at least, and also serglycin, perlecan, dermatan sulfate, keratan sulfate, and/or GPIIb/IIIa. Moreover, the P selectin-defined subset of alpha granules attracts GAG-binding compounds with weaker affinities (i.e., a higher Kd) for GAG and the von Willebrand factor (VWF)-defined subset of alpha granules houses proteins with strong affinity (i.e., higher Kd) interactions with chondroitin sulfate.

Additionally, the present invention is based, in part, on the surprising discovery that an alpha granules' cargo is not released en mass upon aggregation and coagulation. Instead, angiogenesis growth stimulators or inhibitors are released in a spatially- and temporally controlled manner, in response to specific stimuli, such as the local level of thrombin (and/or matrix metalloproteinase (MMP), peroxidase, phosphohydrolase, plasmin, or a plasmin such as tissue plasminogen activator (tPA)). Using thrombin as an example, the early reacting subset of alpha granules, which are labeled with P-selectin, release their contents immediately upon vascular injury (e.g., low thrombin conditions) and when PAR1 (the high-affinity thrombin receptor) was engaged; in contrast, the late reacting subset of alpha granules, which are labeled with vWF factor, release their contents when engaged by PAR4 (i.e., the low affinity thrombin receptor).

Accordingly, the present invention takes advantage of platelets' natural ability to target a breach in a blood vessel's endothelial layer. In the context of cancers, this allows a platelet's cargo to be delivered to a tumor site. Importantly, according to the present disclosure, a platelet's distinct alpha granules are beforehand loaded with two or more agents and these agents are delivered, with specificity, to the provisional matrix formed at the tumor site. Importantly, the present invention the two more agents, which are loaded into distinct alpha granule types, are released from the provisional matrix by tissue proteases in a meticulous - temporally and spatially controlled—enzymatic action.

There are two main GAGs in platelets: heparan sulfate and chondroitin sulfate. Heparan sulfate (HS) is a linear copolymer of uronic acid 1→4 linked to glucosamine but with a highly variable structure. The d-glucuronic acid predominates in HS, although substantial amounts of l-iduronic acid can be present. In comparison to heparin, HS is much less substituted in sulfo groups.

Heparin is highly heterogeneous linear, polydisperse polysaccharide consisting of repeating units of 1→4-linked pyranosyluronic acid and 2-amino-2-deoxyglucopyranose (glucosamine) residues. The uronic acid residues typically consist of 90% 1-idopyranosyluronic acid (l-iduronic acid) and 10% d-glucopyranosyluronic acid (d-glucuronic acid). The amino group of the glucosamine residue may be substituted with an acetyl or sulfo group or unsubstituted. The 3 and 6 positions of the glucosamine residues can either be substituted with an O-sulfo group or unsubstituted. The uronic acid, which can either be l-iduronic or d-glucuronic acid, may also contain a 2-O-sulfo group

Most heparin-binding proteins bind both heparin and heparan sulfate. Both are polydisperse polysaccharides with a heterogeneous saccharide sequences that bind a large number of proteins to a wide range of possible binding sites. Whereas heparin is primarily intracellular, HS proteoglycans (HSPGs) are localized to many cell surfaces and contribute to functions of the extracellular matrix (ECM), e.g., by stabilizing growth factors and protein ligands.

Chondroitin sulfate (CS) is a linear polymer of random sequences of repeated disaccharide units of: 2-acetylamino-2-deoxy-4-0-sulfate-3-0-˜-D-glucopyranurosyl-D-galactose; 2-acetylamino-2-deoxy -6-0-sulfate-3-0-˜-D-glucopyranurosyl-D-galactose; 2-acetylamino-2-deoxy-4,6-0-disulfate-3-0-˜-D-glucopyranurosyl-D-galactose; and 2-acety lamino-2-deoxy-6-0-sulfate-3-0-˜-2′-0-sulfate-D-glucopyranurosyl-D-galactose. Each monosulfated disaccharide unit has a molecular weight of 500-600 g/mol and its total weight is 5-50 kDa. The volume of a molecule of chondroitin sulfate is much larger in solution than in dehydrated solid because it has large number of negative charges; in solution, the negative charges on the variable branches repel each other and force the molecule into an extended conformation. As such, there are numerous ligand-binding sites on a CS molecule.

Novel, non-natural, GAG-binding peptides are useful in the compounds and methods of the present disclosure, as they are essential for the loading of cargo into the alpha granules of platelets. The GAG-binding peptides of the present disclosure are chemically or enzymatically linked (directly or indirectly) to an agent or genetically expressed to produce a fusion protein containing the agent and the binding peptide. The GAG-binding peptide and the coupled agent retain their function in the new compound or fusion product. Thus, the new compound or fusion product is capable of being selectively loaded into a specific alpha granule type of a platelet.

Notably, the loaded platelets of the present disclosure remain in a resting, fully functional platelet, rather than becoming activated by the loading process which would make the platelets pro-coagulant.

### Glycosaminoglycan (GAG)-Binding Peptide

The glycosaminoglycan (GAG)-binding peptide of the present disclosure are characterized by the presence of positively charged basic amino acids that form ion pairs with spatially defined negatively charged sulfo or carboxyl groups on a GAG chain. For example, Heparan sulfate (HS) has an average of two negative charges per disaccharide provided by sulfo and carboxyl groups; thus, the most common type of interaction between HS and proteins is ionic, even though some other non-electrostatic interactions such as hydrogen bonding and hydrophobic interactions may also contribute to the stability of the complexes. It was believed that the highly anionic nature of GAGs leads to nonspecific binding. However, in the alpha granules of platelets, a GAG-binding peptide's binding to HS or chondroitin sulfate (CS) in the specific alpha granule subsets occurs at high specificity. This interaction is facilitated by matching the GAG binding affinity and the GAG-binding peptide. The GAG-peptide interaction depends, in part, on the defined patterns and orientations of the sulfo and carboxyl groups along the polysaccharide sequence in the polymer, and a correct pattern of basic amino acids in the GAG-binding peptide to ensure the appropriate affinity and specificity of the complex.

Electrostatic interactions play a major role in the GAG-peptide interaction, and the position of basic amino acids such as arginine and lysine within the GAG-binding peptide's binding sequence is relevant. A number of studies have been undertaken to determine whether there is a consensus sequence of basic amino acids arranged in a specific way in the GAG-binding sites. For example, a comparison of heparin-binding sites from four proteins: apolipoprotein B, apolipoprotein E, vitronectin, and platelet factor 4 showed that these regions are characterized by two consensus sequences of amino acids: XBBXBX and XBBBXXBX, where B is a basic residue and X is a hydropathic residue. Molecular modeling studies showed that the sequence XBBXBX modeled in a β-strand conformation orients the basic amino acids on one face of the β-strand and the hydropathic residues pointing back into the protein core. Similarly, when the sequence XBBBXXBX is folded into an α-helix, the basic amino acids are displayed on one side of the helix. While some heparin-binding proteins include this consensus sequence, there are others that do not. As such, a structural motif in which the basic residues are close in space, but not necessarily close in the primary amino acid sequence, may also bind heparin.

Heparin-binding sites frequently contain clusters of one, two, or three basic amino acids (XBnX, where n=1, 2, or 3). Spacing of such clusters with one or two non-basic residues (BXmB, where m=1 or 2) is observed in natural proteins; this is consistent with the observation that heparin-binding proteins usually bind HS in biological systems. Because the charge density of HS is lower, optimal protein binding may involve spaced clusters of basic amino acids. Arginine and lysine are the most frequent residues in heparin- and HS-binding proteins. Although both amino acids have a positive charge at physiological pH, arginine binds heparin ˜2.5× more tightly. Arginine forms more stable hydrogen bonds as well as stronger electrostatic interactions with sulfo groups. Non-basic residues might also play an important role in heparin-protein interactions. Among them, serine and glycine have been found to be the most frequent non-basic residues in heparin-binding peptides. Both have small side chains, providing minimal steric constrains and good flexibility for peptide interaction with GAG.

The present invention is based, in part, on a novel, non-natural, glycosaminoglycan (GAG)-binding peptides. The GAG-binding peptides of the present disclosure are capable of binding a GAG in an alpha granule of a platelet. In embodiments, a GAG-binding peptide binds a GAG through electrostatic interactions.

In embodiments, the GAG-binding peptide binds to chondroitin sulfate (CS) and/or heparan sulfate (HS). In embodiments, the GAG-binding peptide preferentially binds to CS. In embodiments, the GAG-binding peptide preferentially binds to chondroitin sulfate A (CSA).

In embodiments, a first GAG-binding peptide preferentially binds to chondroitin sulfate (CS) and a second GAG-binding peptide preferentially binds to heparan sulfate (HS). In embodiments, the first GAG-binding peptide preferentially binds to chondroitin sulfate A (CSA) and does not preferably bind to heparan sulfate (HS).

In embodiments, the GAG-binding peptide binds to heparan sulfate (HS), serglycin, perlecan, dermatan sulfate, keratan sulfate, and/or GPIIb/IIIa. In embodiments, the GAG-binding peptide does not preferentially bind to heparan sulfate (HS), serglycin, perlecan, dermatan sulfate, keratan sulfate, and/or GPIIb/IIIa. In embodiments, the GAG-binding peptide does not bind, does not detectably bind, does not substantially bind, or binds with low affinity to HS, serglycin, perlecan, dermatan sulfate, keratan sulfate, and/or GPIIb/IIIa.

In embodiments, the GAG-binding peptide remains bound to a CS-containing column when exposed to about 1N NaCl. In embodiments, the GAG-binding peptide remains bound to a CS-containing column when exposed to about 2N NaCl. In embodiments, the GAG-binding peptide is unbound to a CS-containing column when exposed to about 3N NaCl.

In embodiments, the GAG-binding peptide is unbound to an HS-containing column, a serglycin-containing column, perlecan-containing column, dermatan sulfate-containing column, keratan sulfate-containing column, and/or GPIIb/IIIa-containing column when exposed to NaCl of between about 0.001N and about 0.01N. In embodiments, the GAG-binding peptide is unbound to an HS-containing column, a serglycin-containing column, perlecan-containing column, dermatan sulfate-containing column, keratan sulfate-containing column, and/or GPIIb/IIIa-containing column when exposed to NaCl of at least about 0.1N. In embodiments, the GAG-binding peptide is unbound to an HS-containing column, a serglycin-containing column, perlecan-containing column, dermatan sulfate-containing column, keratan sulfate-containing column, and/or GPIIb/IIIa-containing column when exposed to NaCl of at least about 1N.

In embodiments, the GAG-binding peptide is between about 8 amino acids and about 14 amino acids in length.

In embodiments, the GAG-binding peptide comprises at least one charged amino acid.

In embodiments, the GAG-binding peptide comprises at least one proline, arginine, and/or isoleucine.

Illustrative GAG-binding peptides comprise one of the following amino acid sequences:

(SEQ ID NO: 1)
ERRIWFPYRRF;
(SEQ ID NO: 2)
RFRWPYRIREF;
(SEQ ID NO: 3)
ARRIWFPYRRF;
(SEQ ID NO: 4)
EARIWFPYRRF;
(SEQ ID NO: 5)
ERAIWFPYRRF;
(SEQ ID NO: 6)
ERRAWFPYRRF;
(SEQ ID NO: 7)
ERRIAFPYRRF;
(SEQ ID NO: 8)
ERRIWAPYRRF;
(SEQ ID NO: 9)
ERRIWFAYRRF;
(SEQ ID NO: 10)
ERRIWFPARRF;
(SEQ ID NO: 11)
ERRIWFPYARF;
(SEQ ID NO: 12)
ERRIWFPYRAF;
and
(SEQ ID NO: 13)
ERRIWFPYRRA.

In embodiments, the GAG-binding peptide comprises an amino acid sequence that is at least about 70% identical to one of SEQ ID NO: 1 to SEQ ID NO: 13, is at least about 80% identical to one of SEQ ID NO: 1 to SEQ ID NO: 13, or is at least about 90% identical to one of SEQ ID NO: 1 to SEQ ID NO: 13.

Without wishing to be bound to theory, it appears that the basic residues (e.g., arginines) are important in defining the GAG-binding peptide's properties and the hydropathic residues provide stabilization.

The GAG-binding peptide may comprise a charged amino acid at position 1, position 4, position 7, or position 9 with respect to any one of SEQ ID NO: 1 to SEQ ID NO: 13.

In embodiments, the GAG-binding peptide comprises a proline, arginine, and/or isoleucine at position 1, position 4, position 7, and/or position 9 with respect to any one of SEQ ID NO: 1 to SEQ ID NO: 13. As examples, the GAG-binding peptide comprises a proline, arginine and/or isoleucine at position 1, position 4, position 7, and position 9; the GAG-binding peptide comprises a proline, arginine and/or isoleucine at position 1; the GAG-binding peptide comprises a proline, arginine and/or isoleucine at position 1 and position 4; the GAG-binding peptide comprises a proline, arginine and/or isoleucine at position 1, position 4, and position 7, and/or position 9; the GAG-binding peptide comprises a proline, arginine and/or isoleucine at position 1, position 4, position 7, and position 9; the GAG-binding peptide comprises a proline, arginine and/or isoleucine at position 1 and position 7; the GAG-binding peptide comprises a proline, arginine and/or isoleucine at position 1 and position 4 and position 9; the GAG-binding peptide comprises a proline, arginine and/or isoleucine at position 1 and position 9; and any combination therebetween. The GAG-binding peptide may comprise a proline at position 1, position 4, position 7, and position 9; the GAG-binding peptide may comprise an arginine at position 1, position 4, position 7, and position 9; the GAG-binding peptide may comprise an isoleucine at position 1, position 4, position 7, and position 9; the GAG-binding peptide may comprise a proline at position 1, and arginines at position 4, position 7, and position 9; the GAG-binding peptide may comprise a proline at position 1, arginines at position 4 and position 7, and an isoleucine at position 9; the GAG-binding peptide may comprise a proline at position 1, an arginine at position 4, and an isoleucine at position 9; or the GAG-binding peptide may comprise an arginine at position 4 and an proline at position 9. Any combinations of proline, arginine, and/or isoleucine at position 1, position 4, position 7, and/or position 9 is encompassed by the present disclosure.

In embodiments, the GAG-binding peptide comprises at least 10 amino acids. In embodiments, the GAG-binding peptide comprises 11 amino acids. In embodiments, the GAG-binding peptide consists of 11 amino acids.

In embodiments, the GAG-binding peptide comprises an amino acid sequence that is at least about 90% identical to SEQ ID NO: 1 or to SEQ ID NO:2.

In embodiments, the GAG-binding peptide comprises an amino acid sequence of one of SEQ ID NO: 1 to SEQ ID NO: 13.

In embodiments, the GAG-binding peptide comprises an amino acid sequence of SEQ ID NO: 1 or SEQ ID NO:2.

In embodiments, the GAG-binding peptide consists of the amino acid sequence of one of SEQ ID NO: 1 to SEQ ID NO: 13.

The invention provides methods for optimizing GAG-binding peptides by producing a variant GAG-binding peptides, e.g., by including deletions, mutations, insertions, or post-translational modifications, in a herein disclosed GAG-binding peptide's amino acid sequence.

A variant may differ from a GAG-binding peptide of SEQ ID NO: 1 to SEQ ID NO: 13 at one amino acid position, as long as the variant GAG-binding peptide retains its function.

A variant may differ from a GAG-binding peptide of SEQ ID NO: 1 to SEQ ID NO: 13 at two amino acid positions, as long as the variant GAG-binding peptide retains its function.

A variant may differ from a GAG-binding peptide of SEQ ID NO: 1 to SEQ ID NO: 13 at three amino acid positions, as long as the variant GAG-binding peptide retains its function.

A variant may differ from a GAG-binding peptide of SEQ ID NO: 1 to SEQ ID NO: 13 at four amino acid positions, as long as the variant GAG-binding peptide retains its function.

A variant may differ from a GAG-binding peptide of SEQ ID NO: 1 to SEQ ID NO: 13 at five amino acid positions, as long as the variant GAG-binding peptide retains its function.

A variant may differ from a GAG-binding peptide of SEQ ID NO: 1 to SEQ ID NO: 13 at more than five amino acid positions, as long as the variant GAG-binding peptide retains its function.

In embodiments, the amino acid differences may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above. A GAG-binding peptide may be modified by including chemical alterations such as acetylation, carboxylation, phosphorylation, or glycosylation.

Accordingly, the present disclosure provides methods for characterizing and optimizing (e.g., increasing affinity) GAG-binding peptides directed against various glycosaminoglycans. The optimized GAG-binding peptides provided by the present disclosure may be directed to glycosaminoglycans present in alpha granules of platelets. Illustrative glycosaminoglycans which are present in alpha granules of platelets include chondroitin sulfate, heparan sulfate, serglycin, perlecan, dermatan sulfate, keratan sulfate, and GPIIb/IIIa. Any of the optimized GAG-binding peptides may be included in a composition of the present disclosure; any of the compositions may be loaded into a platelet, e.g., for inclusion in a pharmaceutical composition and/or for treating a disease or disorder.

### Compounds and Agents

As disclosed herein, distinct alpha granule types of platelets can selectively and actively (i.e., against a concentration gradient) sequester angiogenesis, growth, and inflammation regulating proteins. The present disclosure is based on the discovery that proteins are taken up by platelets and segregated into subsets of alpha granules based on their affinity for glycosaminoglycans (GAGs): predominantly heparan sulfate (HS) and chondroitin sulfate (CS). The long, linear, negatively charged chains of these GAGS provide not only structural support to the alpha granules but also explain the functional subsets of alpha granules. The two main GAGs present in platelets (i.e., HS and CS) differ mainly in the number of disaccharides found in the individual chains. Heparan sulfate is small (15-30 disaccharides/side chain), whereas chondroitin sulfate has many binding sites and has up to 250 disaccharides/side chain Both are distinct from the large, stiff, GAGs such as hyaluronate (up to 50,000 disaccharide/GAG side chain), which functions to maintain the structure and integrity of cartilage and bone. The diversity of the GAGs in platelets is crucial for their function, with the shorter side chains of the heparan sulfate and the weaker binding allowing for early release of P-selectin granules; whereas, the tighter, longer chain binding allows for late release of vWF granules. These features are exploited in the present invention for sequential release of compounds.

The present invention comprises novel, non-naturally occurring platelet anchoring glycosaminoglycan (GAG)-binding peptide which bind CS, at least, and with a very high affinity and bind HS with, at least, moderate affinity. When linked to an agent in a compound of the present disclosure, the GAG-binding peptide facilitates the “loading” of the agents into the alpha granules of platelets. Because platelets continuously circulate and adhere to sites of abnormal endothelium, the compounds of the present disclosure are widely applicable to a variety of pathological conditions.

An aspect of the present disclosure is a composition, e.g., for treating a disease or disorder. The composition comprises a first compound comprising a first agent and a first polypeptide, wherein the first polypeptide comprises a first glycosaminoglycan (GAG)-binding peptide which is capable of binding a GAG in a first alpha granule type of a platelet; and a second compound comprising a second agent and a second polypeptide, wherein the second polypeptide comprises a second glycosaminoglycan (GAG)-binding peptide which is capable of binding a GAG in a second alpha granule type of the platelet.

In embodiments, the first GAG-binding peptide preferentially binds to chondroitin sulfate (CS) and the second GAG-binding peptide preferentially binds to heparan sulfate (HS).

In embodiments, the first GAG-binding peptide preferentially binds to chondroitin sulfate A (CSA) and does not preferably bind to heparan sulfate (HS).

In embodiments, the first alpha granule type is a P-selectin associated granule and the second alpha granule type von Willebrand factor (VWF) associated granule.

In embodiments, the contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin derivative such as tissue plasminogen activator (tPA).

In embodiments, the contents of the first alpha granule type are released at a lower concentration of thrombin than the concentration of thrombin needed to provide release of the contents of the second alpha granule type.

In embodiments, the contents first alpha granule type is released before the contents of the second alpha granule type are released.

In embodiments, the first and the second GAG-binding peptides are each between about 8 amino acids and about 14 amino acids in length.

In embodiments, one or both of the first and the second GAG-binding peptides comprises at least one charged amino acid. In some cases, both of the first and the second GAG-binding peptides comprise at least one charged amino acid.

In embodiments, one or both of the first and the second GAG-binding peptides comprises at least one proline, arginine, and/or isoleucine. In some cases, both of the first and the second GAG-binding peptides comprise at least at least one proline, arginine, and/or isoleucine.

In embodiments, the first and the second GAG-binding peptides independently comprise an amino acid sequence that is at least about 70% identical to one of SEQ ID NO: 1 to SEQ ID NO: 13.

In embodiments, the first and the second GAG-binding peptides independently comprise an amino acid sequence that is at least about 80% identical to one of SEQ ID NO: 1 to SEQ ID NO: 13.

In embodiments, the first and the second GAG-binding peptides independently comprise an amino acid sequence that is at least about 90% identical to one of SEQ ID NO: 1 to SEQ ID NO: 13.

In embodiments, the first and the second GAG-binding peptides independently comprise a charged amino acid at position 1, position 4, position 7, or position 9 with respect to any one of SEQ ID NO: 1 to SEQ ID NO: 13.

In embodiments, the first and the second GAG-binding peptides independently comprise a proline, arginine, and/or isoleucine at position 1, position 4, position 7, and/or position 9 with respect to any one of SEQ ID NO: 1 to SEQ ID NO: 13.

In embodiments, the first and the second GAG-binding peptides independently comprise at least 10 amino acids.

In embodiments, the first and/or the second GAG-binding peptides independently comprise 11 amino acids.

In embodiments, the first and the second GAG-binding peptides independently consist of 11 amino acids.

In embodiments, the first and the second GAG-binding peptides independently comprise the amino acid sequence of one of SEQ ID NO: 1 to SEQ ID NO: 13.

In embodiments, the first GAG-binding peptide comprises an amino acid sequence that is at least about 90% identical to SEQ ID NO: 1 and the second GAG-binding peptide comprises an amino acid sequence that is at least about 90% identical to SEQ ID NO: 2. In some cases the first GAG-binding peptide comprises the amino acid sequence of SEQ ID NO: 1 and the second GAG-binding peptide comprises the amino acid sequence of SEQ ID NO: 2. The first GAG-binding peptide may consist of the amino acid sequence of SEQ ID NO: 1 and the second GAG-binding peptide may consist of the amino acid sequence of SEQ ID NO: 2.

In embodiments, the first polypeptide consists of the first GAG-binding peptide and the second polypeptide consists of the second GAG-binding peptide.

In embodiments, the N-terminal of the first polypeptide is directly or indirectly linked to the first agent and/or the N-terminal of the second polypeptide is directly or indirectly linked to second first agent.

In embodiments, the C-terminal of the first polypeptide is directly or indirectly linked to the first agent and/or the C-terminal of the second polypeptide is directly or indirectly linked to second first agent.

In any herein disclosed aspect or embodiment, an agent (first, second, or third) and GAG-binding peptide (first, second, or third) may be directly linked or they may be linked via a moiety referred to as a linker. A linker refers to a chemical moiety comprising a covalent bond or a chain of atoms that covalently attaches an agent to a GAG-binding peptide. Linkers include a divalent radical such as an alkylene, an arylene, a heteroarylene, moieties such as: —(CR2) nO(CR2) n-, a polymer of repeating units of alkyloxy (e.g., polyethylenoxy, polyethylene glycol (PEG), polymethyleneoxy) and alkylamino (e.g., polyethyleneamino, Jeffamine™); and diacid ester and amides including succinate, succinamide, diglycolate, malonate, and caproamide. In embodiments, the linker comprises a chain of amino acids. In embodiments, the amino acid chain linker is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the amino acid chain linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In embodiments, the amino acid chain linker is between about 15 amino acids and about 3 amino acids, e.g., between about 10 and 5 amino acids.

In embodiments, the first agent is indirectly linked to the first polypeptide via a first linker and/or the second agent is indirectly linked to the second polypeptide via a second linker. In some cases, the first linker and/or the second each comprise one or more atoms. The first linker and/or the second may each comprise a polymer of repeating units. The first linker and/or the second linker may each comprise a chain of amino acids.

In embodiments, the first agent is directly linked to the first polypeptide and/or the second agent is directly linked to the second polypeptide.

In embodiments, the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

In embodiments, the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet. In some cases, the first agent and/or the second agent comprises an antibody or a fluorescent moiety.

Illustrative antibodies (or fragments thereof) useful in the present invention include 3F8, 8H9, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Anatumomab mafenatox, Andecaliximab, Anetumab ravtansine, Anifrolumab, Anrukinzumab (IMA-638), Apolizumab, Aprutumab ixadotin, Arcitumomab, Ascrinvacumab, Aselizumab, Atezolizumab, Atidortoxumab, Atinumab, Atorolimumab, Avelumab, Azintuxizumab vedotin, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab mertansine, Bleselumab, Blinatumomab, Blontuvetmab, Blosozumab, BMS 936559, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, CR6261, Crenezumab, Crizanlizumab, Crotedumab, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan and 90Y-Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Inolimomab, Inotuzumab ozogamicin, Intetumumab, Iomab-B, Ipilimumab, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Loncastuximab tesirine, Lorvotuzumab mertansine, Losatuxizumab vedotin, Lucatumumab, Lulizumab pegol, Lumiliximab, Lumretuzumab, Lupartumab amadotin, Lutikizumab, Mapatumumab, Margetuximab, Marstacimab, Maslimomab, Matuzumab, Mavrilimumab, Mepolizumab, Metelimumab, Milatuzumab, Minretumomab, Mirikizumab, Mirvetuximab soravtansine, Mitumomab, MK-3475, Modotuximab, Mogamulizumab, Monalizumab, Morolimumab, Mosunetuzumab, Motavizumab, Moxetumomab pasudotox, MPDL328OA, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Ravagalimab, Ravulizumab, Raxibacumab, Refanezumab, Regavirumab, Relatlimab, Remtolumab, Reslizumab, Rilotumumab, Rinucumab, Risankizumab, Rituximab, Rivabazumab pegol, Rmab, Robatumumab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, SGN-CD19A, SHP647, Sibrotuzumab, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tigatuzumab, Tildrakizumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab and 131I-tositumomab, Tovetumab, Tralokinumab, Trastuzumab, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab (IMAB362, Claudiximab), and Zolimomab aritox.

Illustrative antibodies (or fragments thereof) that have met or have pending regulatory approval and are useful in the present invention include Muromonab-CD3 (ORTHOCLONE OKT3), Efalizumab (RAPTIVA), Tositumomab-I131 (BEXXAR), Nebacumab (CENTOXIN), Edrecolomab (PANOREX), Catumaxomab (REMOVAB), Daclizumab (ZINBRYTA; ZENAPAX), Abciximab (REOPRO), Rituximab (MABTHERA, RITUXAN), Basiliximab (SIMULECT), palivizumab (SYNAGIS), Infliximab (REMICADE), Trastuzumab (HERCEPTIN), Adalimumab (HUMIRA), Ibritumomab tiuxetan (ZEVALIN), Omalizumab (XOLAIR), Cetuximab (ERBITUX), Bevacizumab (AVASTIN), Natalizumab (TYSABRI), Panitumumab (VECTIBIX), Ranibizumab (LUCENTIS), Eculizumab (SOLIRIS), Certolizumab pegol (CIMZIA), Ustekinumab (STELARA), Canakinumab (ILARIS), Golimumab (SIMPONI), Ofatumumab (ARZERRA), Tocilizumab (ROACTEMRA, ACTEMRA), Denosumab (PROLIA), Belimumab (BENLYSTA), Ipilimumab (YERVOY), Brentuximab vedotin (ADCETRIS), Pertuzumab (PERJETA), Ado-trastuzumab emtansine (KADCYLA), Raxibacumab), Obinutuzumab (GAZYVA, GAZYVARO), Siltuximab (SYLVANT), Ramucirumab (CYRAMZA), Vedolizumab (ENTYVIO), Nivolumab (OPDIVO), Pembrolizumab (KEYTRUDA), Blinatumomab (BLINCYTO), Alemtuzumab (LEMTRADA; MABCAMPATH, CAMPATH-1H), Evolocumab (REPATHA), Idarucizumab (PRAXBIND), Necitumumab (PORTRAZZA), Dinutuximab (UNITUXIN), Secukinumab (COSENTYX), Mepolizumab (NUCALA), Alirocumab (PRALUENT), Daratumumab (DARZALEX), Elotuzumab (EMPLICITI), Ixekizumab (TALTZ), Reslizumab (CINQAERO, CINQAIR), Olaratumab (LARTRUVO), Bezlotoxumab (ZINPLAVA), Atezolizumab (TECENTRIQ), Obiltoxaximab (ANTHIM), Brodalumab (SILIQ, LUMICEF), Dupilumab (DUPIXENT), Inotuzumab ozogamicin (BESPONSA), Guselkumab (TREMFYA), Sarilumab (KEVZARA), Avelumab (BAVENCIO), Emicizumab (HEMLIBRA), Ocrelizumab (OCREVUS), Benralizumab (FASENRA), Durvalumab (IMFINZI), Gemtuzumab ozogamicin (MYLOTARG), Erenumab, erenumab-aooe (AIMOVIG), Galcanezumab, galcanezumab-gnlm (EMGALITY), Burosumab, burosumab-twza (CRYSVITA), Lanadelumab, lanadelumab-flyo (TAKHZYRO), Mogamulizumab, mogamulizumab-kpkc (POTELIGEO), Tildrakizumab; tildrakizumab-asmn (ILUMYA), Fremanezumab, fremanezumab-vfrm (AJOVY), Ravulizumab, ravulizumab-cwvz (ULTOMIRIS), Cemiplimab, cemiplimab-rwlc (LIBTAYO), Ibalizumab, ibalizumab-uiyk (TROGARZO), Emapalumab, emapalumab-lzsg (GAMIFANT), Moxetumomab pasudotox, moxetumomab pasudotox-tdfk (LUMOXITI), Caplacizumab, caplacizumab-yhdp (CABLIVI), Risankizumab, risankizumab-rzaa (SKYRIZI), Polatuzumab vedotin, polatuzumab vedotin-piiq (POLIVY), Romosozumab, romosozumab-aqqg (EVENITY), Brolucizumab, brolucizumab-dbll (BEOVU), Crizanlizumab; crizanlizumab-tmca (ADAKVEO), Enfortumab vedotin, enfortumab vedotin-ejfv (PADCEV), [fam-]trastuzumab deruxtecan, fam-trastuzumab deruxtecan-nxki (ENHERTU), Teprotumumab, teprotumumab-trbw (TEPEZZA), Eptinezumab, eptinezumab-jjmr (VYEPTI), Isatuximab, isatuximab-irfc (SARCLISA), Sacituzumab govitecan; sacituzumab govitecan-hziy (TRODELVY), Inebilizumab; inebilizumab-cdon (UPLIZNA), Satralizumab (ENSPRYNG), Dostarlimab (TSR-042), Sutimlimab (BIVV009), Leronlimab, Narsoplimab, Tafasitamab, REGNEB3, Naxitamab, Oportuzumab monatox, Belantamab mafodotin, Margetuximab, Tanezumab, Teplizumab, Aducanumab, Evinacumab, Tralokinumab, and Omburtamab.

A fragment of an antibody will comprise, at least, the antigen-binding domain of an above-mentioned antibody. In embodiments, the antigen-binding domain is an antibody, an antibody fragment, an scFv, a Fv, a Fab, a (Fab′)2, a single domain antibody (SDAB), a VH or VL domain, or a camelid VHH domain, e.g., a human scFv, human Fv, human Fab, human (Fab′)2, human single domain antibody (SDAB), or human VH or VL domain or a humanized scFv, humanized Fv, humanized Fab, humanized (Fab′)2, humanized single domain antibody (SDAB), or humanized VH or VL domain.

Illustrative chemotherapeutic agents useful in the present invention include 2,3,4,5,6-pentafluoro-N-(3-fluoro-4-methoxyphenyl)benzene sulfonamide, 3′,4′-didehydro-4′-deoxy-8′-norvin-caleukoblastine, 5-FU (Fluorouracil), Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, ADE, Adriamycin (Doxorubicin), Afatinib Dimaleate, Afinitor (Everolimus), Afinitor Difsperz (Everolimus), Akynzeo (Netupitant and palonosetron), Aldara (Imiquimod), Aldesleukin, Alecensa (Alectinib), Alectinib, Alimta (PEMETREXED), Aliqopa (Copanlisib Hydrochloride), Alkeran (Melphalan), Aloxi (palonosetron Hydrochloride), Altretamine, Alunbrig (Brigatinib), Ambochlorin (Chlorambucil), Amboclorin (Chlorambucil), Amifostine, Aminolevulinic Acid, Anastrozole, Anhydrovinblastine, Aprepitant, Aredia (Pamidronate), Arimidex (Anastrozole), Aromasin (Exemestane), Arranon (Nelarabine), Arsenic Trioxide, Asparaginase Erwinia chrysanthemi, Auristatin, Axicabtagene Ciloleucel, Axitinib, Azacitidine, BEACOPP, Becenum (Carmustine), Beleodaq (Belinostat), Belinostat, Bendamustine Hydrochloride, BEP, Bexarotene, Bicalutamide, BiCNU (Carmustine), Blenoxane (Bleomycin), BMS184476, Bortezomib, Bosulif (Bosutinib), Bosutinib, Brigatinib, BuMel, Busulfan, Busulfex (Busulfan)C, Cabazitaxel, Cabometyx (Cabozantinib), Cabozantinib-S-Malate, CAF, Calquence (Acalabrutinib), Camptosar (Irinotecan Hydrochloride), Capecitabine, CAPOX, Caprelsa (Vandetanib), Carac (Fluorouracil-Topical), Carboplatin, Carboplatin-Taxol, Carfilzomib, Carmubris (Carmustine), Carmustine, Casodex (Bicalutamide), Cachectin, CeeNU (Lomustine), CEM, Cemadotin, Ceritinib, Cerubidine (Daunorubicin), Cervarix (Recombinant HPV Bivalent Vaccine), CEV, Chlorambucil, Chlorambucil-Prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib), Copanlisib Hydrochloride, COPDAC, COPP, COPP-ABV, Cosmegen (Dactinomycin), Cotellic (Cobimetinib), Cryptophycin, Crizotinib, CVP, Cyclophosphamide, Cyfos (Ifosfamide), Cytarabine, Cytarabine Liposome, Cytosar-U (Cytarabine), Cytoxan (Cyclophosphamide), Cytoxan (Cytoxan), Dabrafenib, Dacarbazine, Dacogen (Decitabine), Dactinomycin, Dasatinib, Daunorubicin Hydrochloride, Daunorubicin Hydrochloride and Cytarabine Liposome, DaunoXome (Daunorubicin Lipid Complex), Decadron (Dexamethasone), Decitabine, Defibrotide Sodium, Defitelio (Defibrotide Sodium), Degarelix, Denileukin Diftitox, DepoCyt (Cytarabine Liposome), Dexamethasone, Dexamethasone Intensol (Dexamethasone), Dexpak Taperpak (Dexamethasone), Dexrazoxane Hydrochloride, Docefrez (Docetaxel), Docetaxel, Docetaxol, Dolastatin, Doxetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Droxia (Hydroxyurea), DTIC (Decarbazine), DTIC-Dome (Dacarbazine), Efudex (Fluorouracil-Topical), Eligard (Leuprolide), Elitek (Rasburicase), Ellence (Ellence (epirubicin)), Eloxatin (Oxaliplatin), Elspar (Asparaginase), Eltrombopag Olamine, Emcyt (Estramustine), Emend (Aprepitant), Enasidenib Mesylate, Enzalutamide, Epirubicin Hydrochloride, EPOCH, Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Eulexin (Flutamide), Evacet (Doxorubicin Hydrochloride Liposome), Everolimus, Evista (Raloxifene Hydrochloride), Evomela (Melphalan Hydrochloride), Exemestane, Fareston (Toremifene), Farydak (Panobinostat), Faslodex (Fulvestrant), FEC, Femara (Letrozole), Filgrastim, Firmagon (Degarelix), Finasteride, FloPred (Prednisolone), Fludara (Fludarabine), Fludarabine Phosphate, Fluoroplex Fluorouracil), Fluorouracil, Flutamide, Folex (Methotrexate), Folex PFS (Methotrexate), FOLFIRI, FOLFIRINOX, FOLFOX, Folotyn (Pralatrexate), FUDR (FUDR (floxuridine)), FU-LV, Fulvestrant, Gardasil (Recombinant HPV Quadrivalent Vaccine), Gardasil 9 (Recombinant HPV Nonavalent Vaccine), Gefitinib, Gemcitabine Hydrochloride, GEMCITABINE-CISPLATIN, GEMCITABINE-OXALIPLATIN, Gemzar (Gemcitabine), Gilotrif (Afatinib Dimaleate), Gilotrif (Afatinib), Gleevec (Imatinib Mesylate), Gliadel (Carmustine), Glucarpidase, Goserelin Acetate, Halaven (Eribulin Mesylate), Hemangeol (Propranolol Hydrochloride), Hexalen (Altretamine), HPV Bivalent Vaccine, Recombinant, HPV Nonavalent Vaccine, Recombinant, HPV Quadrivalent Vaccine, Recombinant, Hycamtin (Topotecan Hydrochloride), Hycamtin (Topotecan), Hydrea (Hydroxyurea), Hydroxyurea, Hydroxyureataxanes, Hyper-CVAD, Ibrance (palbociclib), Ibrutinib, ICE, Iclusig (Ponatinib), Idamycin PFS (Idarubicin), Idarubicin Hydrochloride, Idelalisib, Idhifa (Enasidenib), Ifex (Ifosfamide), Ifosfamide, Ifosfamidum (Ifosfamide), Imatinib Mesylate, Imbruvica (Ibrutinib), Imiquimod, Imlygic (Talimogene Laherparepvec), Inlyta (Axitinib), Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jakafi (Ruxolitinib), JEB, Jevtana (Cabazitaxel), Keoxifene (Raloxifene Hydrochloride), Kepivance (palifermin), Kisqali (Ribociclib), Kyprolis (Carfilzomib), Lanreotide Acetate, Lanvima (Lenvatinib), Lapatinib Ditosylate, Lenalidomide, Lenvatinib Mesylate, Lenvima (Lenvatinib Mesylate), Letrozole, Leucovorin Calcium, Leukeran (Chlorambucil), Leukine (Sargramostim), Leuprolide Acetate, Leustatin (Cladribine), Levulan (Aminolevulinic Acid), Liarozole, Linfolizin (Chlorambucil), LipoDox (Doxorubicin Hydrochloride Liposome), Lomustine, Lonidamine, Lonsurf (Trifluridine and Tipiracil), Lupron (Leuprolide), Lynparza (Olaparib), Lysodren (Mitotane), Marqibo (Vincristine Sulfate Liposome), Marqibo Kit (Vincristine Lipid Complex), Matulane (Procarbazine), Mechlorethamine Hydrochloride, Megace (Megestrol), Megestrol Acetate, Mekinist (Trametinib), Melphalan, Melphalan Hydrochloride, Mercaptopurine, Mesnex (Mesna), Metastron (Strontium-89 Chloride), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), Mivobulin isethionate, MOPP, Mostarina (Prednimustine), Mozobil (Plerixafor), Mustargen (Mechlorethamine), Mutamycin (Mitomycin), Myleran (Busulfan), Mylosar (Azacitidine), Nanoparticle Paclitaxel (Paclitaxel Albumin-stabilized Nanoparticle Formulation), Navelbine (Vinorelbine), Nelarabine, Neosar (Cyclophosphamide), Neratinib Maleate, Nerlynx (Neratinib), Netupitant and palonosetron Hydrochloride, Neulasta (filgrastim), Neulasta (pegfilgrastim), Neupogen (filgrastim), Nexavar (Sorafenib), Nilandron (Nilutamide), Nilotinib, Nilutamide, Ninlaro (Ixazomib), Nipent (Pentostatin), Niraparib Tosylate Monohydrate, N,n-dimethyl-l-valyl-l-valyl-n-methyl-l-valyl-l-proly-l-lproline-t-butylamide, Nolvadex (Tamoxifen), Novantrone (Mitoxantrone), Nplate (Romiplostim), Odomzo (Sonidegib), OEPA, OFF, Olaparib, Omacetaxine Mepesuccinate, Onapristone, Oncaspar (Pegaspargase), Oncovin (Vincristine), Ondansetron Hydrochloride, Onivyde (Irinotecan Hydrochloride Liposome), Ontak (Denileukin Diftitox), Onxol (Paclitaxel), OPPA, Orapred (Prednisolone), Osimertinib, Oxaliplatin, Paclitaxel, Paclitaxel Albumin-stabilized Nanoparticle Formulation, PAD, palbociclib, palifermin, palonosetron Hydrochloride, palonosetron Hydrochloride and Netupitant, Pamidronate Disodium, Panobinostat, Panretin (Alitretinoin), Paraplat (Carboplatin), Pazopanib Hydrochloride, PCV, PEB, Pediapred (Prednisolone), Pegaspargase, Pegfilgrastim, Pemetrexed Disodium, Platinol (Cisplatin), PlatinolAQ (Cisplatin), Plerixafor, Pomalyst (Pomalidomide), Ponatinib Hydrochloride, Pralatrexate, Prednimustine, Prednisone, Procarbazine Hydrochloride, Proleukin (Aldesleukin), Promacta (Eltrombopag Olamine), Propranolol Hydrochloride, Purinethol (Mercaptopurine), Purixan (Mercaptopurine), Radium 223 Dichloride, Raloxifene Hydrochloride, Rasburicase, R-CHOP, R-CVP, Reclast (Zoledronic acid), Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Regorafenib, Relistor (Methylnaltrexone Bromide), R-EPOCH, Revlimid (Lenalidomide), Rheumatrex (Methotrexate), Rhizoxin, Ribociclib, R-ICE, Rolapitant Hydrochloride, Romidepsin, Romiplostim, Rpr109881, Rubex (Doxorubicin), Rubidomycin (Daunorubicin Hydrochloride), Rubraca (Rucaparib), Rucaparib Camsylate, Ruxolitinib Phosphate, Rydapt (Midostaurin), Sandostatin (Octreotide), Sandostatin LAR Depot (Octreotide), Sclerosol Intrapleural Aerosol (Talc), Sertenef, Soltamox (Tamoxifen), Somatuline Depot (Lanreotide Acetate), Sonidegib, Sorafenib Tosylate, Sprycel (Dasatinib), STANFORD V, Sterapred (Prednisone), Sterapred DS (Prednisone), Sterile Talc Powder (Talc), Steritalc (Talc), Sterecyst (Prednimustine), Stivarga (Regorafenib), Stramustine phosphate, Streptozocin, Sunitinib Malate, Supprelin LA (Histrelin), Sutent (Sunitinib Malate), Sutent (Sunitinib), Synribo (Omacetaxine Mepesuccinate), Tabloid (Thioguanine), TAC, Tafmlar (Dabrafenib), Tagrisso (Osimertinib), Talc, Talimogene Laherparepvec, Tamoxifen Citrate, Tarabine PFS (Cytarabine), Tarceva (Erlotinib), Targretin (Bexarotene), Tasigna (Decarbazine), Tasigna (Nilotinib), Tasonermin, Taxol (Paclitaxel), Taxotere (Docetaxel), Temodar (Temozolomide), Temozolomide, Temsirolimus, Tepadina (Thiotepa), Thalidomide, Thalomid (Thalidomide), TheraCys BCG (BCG), Thioguanine, Thioplex (Thiotepa), Thiotepa, TICE BCG (BCG), Tisagenlecleucel, Tolak (Fluorouracil-Topical), Toposar (Etoposide), Topotecan Hydrochloride, Toremifene, Torisel (Temsirolimus), Totect (Dexrazoxane Hydrochloride), TPF, Trabectedin, Trametinib, Treanda (Bendamustine hydrochloride), Trelstar (Triptorelin), Tretinoin , Trexall (Methotrexate), Trifluridine and Tipiracil Hydrochloride, Trisenox (Arsenic trioxide), Tykerb (lapatinib), Uridine Triacetate, VAC, Valrubicin, Valstar (Valrubicin Intravesical), Valstar (Valrubicin), VAMP, Vandetanib, Vantas (Histrelin), Varubi (Rolapitant), VeIP, Velban (Vinblastine), Velcade (Bortezomib), Velsar (Vinblastine Sulfate), Vemurafenib, Venclexta (Venetoclax), Vepesid (Etoposide), Verzenio (Abemaciclib), Vesanoid (Tretinoin), Viadur (Leuprolide Acetate), Vidaza (Azacitidine), Vinblastine, Vincasar PFS (Vincristine), Vincrex (Vincristine), Vincristine Sulfate, Vincristine Sulfate Liposome, Vindesine sulfate, Vinflunine, Vinorelbine Tartrate, VIP, Vismodegib, Vistogard (Uridine Triacetate), Voraxaze (Glucarpidase), Vorinostat, Votrient (Pazopanib), Vumon (Teniposide), Vyxeos (Daunorubicin Hydrochloride and Cytarabine Liposome), W, Wellcovorin (Leucovorin Calcium), Wellcovorin IV (Leucovorin), Xalkori (Crizotinib), XELIRI, Xeloda (Capecitabine), XELOX, Xofigo (Radium 223 Dichloride), Xtandi (Enzalutamide), Yescarta (Axicabtagene Ciloleucel), Yondelis (Trabectedin), Zaltrap (Ziv-Aflibercept), Zanosar (Streptozocin), Zarxio (Filgrastim), Zejula (Niraparib), Zelboraf (Vemurafenib), Zinecard (Dexrazoxane Hydrochloride), Ziv-Aflibercept, Zofran (Ondansetron Hydrochloride), Zoladex (Goserelin), Zoledronic Acid, Zolinza (Vorinostat), Zometa (Zoledronic acid), Zortress (Everolimus), Zydelig (Idelalisib), Zykadia (Ceritinib), Zytiga (Abiraterone Acetate), and Zytiga (Abiraterone). Other examples of chemotherapeutic agents can be found in Cancer Principles and Practice of Oncology by V. T. Devita and S. Hellman (editors), 6th edition (Feb. 15, 2001), Lippincott Williams & Wilkins Publishers, the contents of which is incorporated herein by reference in its entirety.

In embodiments, a chemotherapeutic agent, e.g., from the above list, may be included as an agent in a compound of the present disclosure. Alternately, or additionally, a chemotherapeutic agent, e.g., from the above list, may be used in conjunction with a compound of the present disclosure, i.e., in a combination therapy. As examples, a subject may be administered platelets loaded with one or both of a compound comprising a multikinase inhibitor (e.g., regorafenib) as agent and a compound comprising fumagillin as agent, and also administered a chemotherapeutic agent; this combination may be used for treating pancreatic cancer, lung cancer, or colon cancer. A subject may be administered platelets loaded with one or both of a compound comprising an EGFR inhibitor (e.g., Cetuximab) as agent and a compound comprising a multikinase inhibitor (e.g., regorafenib) as an active agent and also administered a chemotherapeutic agent; this may be used for treating lung cancer. Also, subject may be administered platelets loaded with one or both or all three of a compound comprising an EGFR inhibitor (e.g., Cetuximab) as agent, a compound comprising a multikinase inhibitor (e.g., regorafenib) as agent, and a compound comprising an ALK/ROS1/NTRK inhibitor (e.g., crizotinib) as agent and also administered a chemotherapeutic agent; this may be used for treating non-small cell lung cancer.

Illustrative immune checkpoint inhibitors useful in the present invention include full-length or fragments of ligands or receptors for A2AR, B7-H3, B7-H4, BTLA, CD122, CD137, CD27, CD28, CD28, CD40, CTLA-4, GITR, ICOS, ICOS, IDO, KIR, KIR., LAG3, NOX2, OX40, PD-1, SIGLEC7, SIGLEC9, TIM-3, and VISTA.

Illustrative growth factors useful in the present invention include vascular endothelial growth factor (VEGF), basic fibroblast growth factor (bFGF), and platelet-derived growth factor (PDGF), Epidermal Growth Factor (EGF), Hepatocyte Growth Factor (HGF), Insulin-Like Growth Factor (IGF), and an Angiopoietin.

Illustrative growth inhibitors useful in the present invention include angiostatin, endostatin, tumstatin, Thrombospondin-1 (TSP1), Platelet Factor 4 (PF4, CXCL4), and Tissue inhibitors of Metalloproteinases (TIMPs).

Illustrative proteases/proteinases useful in the present invention include Matrix Metalloproteinases (MMPs), thrombin, tissue plasminogen activator (tPA), urokinase, and streptokinase.

Illustrative coagulation factors useful in the present invention include Factor II (thrombin), Antithrombin III (ATIII), Kallikrein, tissue factor (TF), Factor V, Factor VII, Factor VIII, Factor IX, Factor X, Factor XI, and Factor XII, Factor XIII, Fibrinogen, Protein S, Protein C, thrombomodulin, plasminogen, and tissue factor pathway inhibitor (TFPI).

Illustrative lipids or phospholipids useful in the present invention include apolipoprotein E (ApoE), platelet phospholipids, and Sphingosine-1-phosphate (SIP).

Illustrative extracellular matrix proteins useful in the present invention include integrins, fibronectin, laminin, focal adhesion proteins (FAK), vinculin, talin, actin filaments, and collagen.

Illustrative hormones useful in the present invention include insulin, steroid (e.g., estrogen, progesterone, and testosterone, and variants thereof), erythropoietin, thrombopoietin, and thyroid hormone.

Illustrative enzymes useful in the present invention include Heparanase or a Matrix Metalloproteinase (MMP).

Illustrative chemokines/chemoattractants useful in the present invention include Connective Tissue Growth Factor (CTGF), Stromal Cell-derived Factor-1 (SDF-1) (CXCL12), interleukins (IL1, 2, 6, 8), and CD40 Ligand (CD40L, CD154).

Illustrative neurotrophins useful in the present invention include nerve growth factor (NGF), brain-derived neurotrophic factor (BDNF), Neurotrophin-3 (NT-3), and Neurotrophin 4/5 (NT-4/5).

In embodiments, an agent is selected from the following non-exhaustive list which includes useful agents of various classifications: 3-4-(1-formylpiperazin-4-yl)-benzylidenyl-2-indolinone, Abatacept, ABT-869, Acalabrutinib, Afatinib, Aflibercept, Alectinib, Alefacept, AMG 108, Antilymphocyte immunoglobulin (horse), Antithymocyte immunoglobulin (rabbit), Apomab, Asfotase alfa, Asunercept, AVE9633, Axitinib, Belatacept, Bevacizumab zirconium Zr-89, BIIB015, Bivatuzumab, Bosutinib, Brigatinib, Cabozantinib, Canertinib, Capmatinib, Cediranib, Ceritinib, CR002, Crenolanib, Crizotinib, CT-011, Dacomitinib, Dasatinib, Depatuxizumab, Dovitinib, Edratide, Entrectinib, Erdafitinib, Erlotinib, Etanercept, Famitinib, Fedratinib, Firategrast, Flumatinib, Foretinib, Fostamatinib, Gefitinib, Geldanamycin, Genistein, Gilteritinib, Glesatinib, GMA-161, Gremubamab, GS-5745, Human cytomegalovirus immune globulin, Human immunoglobulin G, Human Varicella-Zoster Immune Globulin, Ibritumomab tiuxetan, Ibrutinib, Icotinib, IGN311, Imatinib, Indium In-111 satumomab pendetide, IPH 2101, Labetuzumab govitecan, Lapatinib, Larotrectinib, Lecanemab, Lenvatinib, Lestaurtinib, Lorukafusp alfa, Midostaurin, Mirvetuximab Soravtansine, Mitazalimab, Motesanib, Muromonab, Naptumomab Estafenatox, NAV 1800, Neratinib, Nilotinib, Nintedanib, Osimertinib, Pacritinib, Pazopanib, PD173955, Pexidartinib, Piceatannol, Ponatinib, Radicicol, Radotinib, Regorafenib, RI 624, Rovalpituzumab Tesirine, Rozrolimupab, Ruxolitinib, Saracatinib, Savolitinib, SB-1578, Selpercatinib, Selumetinib, Sorafenib, Sunitinib, Tafasitamab, Tandutinib, TB-402, Technetium Tc-99m arcitumomab, Tesevatinib, TNX-901, Tomaralimab, Tositumomab, Trastuzumab deruxtecan, Tucatinib, Vadastuximab Talirine, Valanafusp alfa, Vandetanib, Vatalanib, Vemurafenib, VS-4718, XmAb 2513, XTL-001, and Zolbetuximab.

In embodiments, the agent is an EGFR inhibitor (e.g., Cetuximab).

In embodiments, the agent is a VEGF inhibitor (e.g., Bevacizumab)

In embodiments, the agent is a PDL1 inhibitor (e.g., Pembrolizumab).

In embodiments, the agent is an FN1 inhibitor (e.g., Ocriplasmin).

In embodiments, the agent is a multikinase inhibitor (e.g., regorafenib).

In embodiments, the agent is a FGFR2 antagonist (e.g., thalidomide).

In embodiments, the agent is thrombin and its analogues.

In embodiments, the agent is a CSF3R agonist (e.g., Filgrastim).

In embodiments, the agent is a PSMB5 inhibitor (e.g., Bortezomib).

In embodiments, the agent is fumagillin.

In embodiments, the agent is an ALK/ROS1/NTRK inhibitor (e.g., crizotinib).

In embodiments, the first agent and/or the second agent is harmful to mammalian cells and/or is toxic to a subject and/or the first agent and/or the second agent is susceptible to degradation when administered directly into the bloodstream of a subject.

In embodiments, the first compound and/or the second compound further comprises a fluorescent moiety.

In embodiments, the first GAG-binding peptide and/or the second GAG-binding peptide also preferentially binds serglycin, perlecan, dermatan sulfate, keratan sulfate, and/or GPIIb/IIIa.

In embodiments, the composition further comprises a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

### Isolated Platelets

Often an agent useful for treating disease or disorders, can be harmful to human cells and/or is toxic to a subject, and especially when administered systemically to the subject. Loading platelets with a compound comprising the harmful agent avoids the unintended and undesirable cellular, tissue, and/or organ damage in the subject. Additionally, certain agents are susceptible to degradation when administered directly into the bloodstream of a subject. Loading platelets with a compound comprising the degradable agent avoids a reduction is concentration of the agent which would occur when administered directly into the bloodstream of a subject; thus, the loaded platelets avoid a reduction in dose (e.g., below an effective dose) when administered to the subject. Together, the loaded platelets provide enrichment of the agent localized to the target site, at a desirable dose and with fewer adverse effects.

The technique of platelet-facilitated delivery of agents has numerous advantages over other targeted delivery systems. Unlike nanoparticle-facilitate delivery, no foreign substances are provided to the subject. Similarly, while liposomal preparations have short shelf life, poor stability, and short in vivo half-life due to phagocytosis by the reticulo-endothelial system (RES), the platelet delivery system of the present disclosure extends the in vivo half-life and does not change the stability and preparation of the original compound. Also, most synthetic homing mechanisms, such as RGD peptides, which target abnormal vasculature, have not achieved the specificity of native platelets. Finally, the use of autologous platelets in the present invention eliminates the risk of another's infectious agents; this increases the safety of the procedure, and the speed of platelet loading (seconds to minutes) without needing to thaw and/or prepare donated and stored platelets. Together, the platelets-facilitated delivery of agents of the present disclosure can readily and easily be translated into the clinic.

Another aspect of the present disclosure is an isolated platelet. The isolated platelet comprises at least one copy of a first compound comprising a first agent and a first polypeptide, wherein the first polypeptide comprises a first glycosaminoglycan (GAG)-binding peptide which is capable of binding a GAG in a first alpha granule type of a platelet; and at least one copy of a second compound comprising a second agent and a second polypeptide, wherein the second polypeptide comprises a second glycosaminoglycan (GAG)-binding peptide which is capable of binding a GAG in a second alpha granule type of the platelet.

In embodiments, the platelet is a synthetic, an allogeneic, an autologous, or a modified heterologous platelet.

In embodiments, the platelet is an autologous platelet.

In embodiments, the platelet is an allogeneic platelet.

In embodiments, the platelet is obtained from platelet rich plasma.

In embodiments, the platelet comprises 1 to 1000 copies of the first compound and 1 to 1000 copies of the second compound. In some cases, the 1 to 1000 copies of the first compound are loaded into a first alpha granule type of a platelet and the 1 to 1000 copies of the second compound are loaded into a second alpha granule type of the platelet. The least one copy of the first compound may be loaded into a second alpha granule type of a platelet and at least one copy of the second compound may be loaded into a first alpha granule type of the platelet.

In embodiments, the first GAG-binding peptide preferentially binds to chondroitin sulfate (CS) and the second GAG-binding peptide preferentially binds to heparan sulfate (HS).

In embodiments, the first GAG-binding peptide preferentially binds to chondroitin sulfate A (CSA) In embodiments, the first alpha granule type is a P-selectin associated granule and the second alpha granule type von Willebrand factor (VWF) associated granule.

In embodiments, the contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin such as tissue plasminogen activator (tPA). PAR1 In embodiments, the contents of the first alpha granule type are released at a lower concentration of thrombin than the concentration of thrombin needed to provide release of the contents of the second alpha granule type.

In embodiments, the contents first alpha granule type is released before the contents of the second alpha granule type are released.

In embodiments, the first and the second GAG-binding peptides are each between about 8 amino acids and about 14 amino acids in length. In some cases, or both of the first and the second GAG-binding peptides comprises at least one charged amino acid. Both the first and the second GAG-binding peptides may comprise at least one charged amino acid.

In embodiments, one or both of the first and the second GAG-binding peptides comprises at least one proline, arginine, and/or isoleucine. In some cases, both of the first and the second GAG-binding peptides comprise at least at least one proline, arginine, and/or isoleucine.

In embodiments, a GAG-binding peptide comprises a proline, arginine, and/or isoleucine at position 1, position 4, position 7, and/or position 9 with respect to any one of SEQ ID NO: 1 to SEQ ID NO: 13. As examples, the GAG-binding peptide comprises a proline, arginine and/or isoleucine at position 1, position 4, position 7, and position 9; the GAG-binding peptide comprises a proline, arginine and/or isoleucine at position 1; the GAG-binding peptide comprises a proline, arginine and/or isoleucine at position 1 and position 4; the GAG-binding peptide comprises a proline, arginine and/or isoleucine at position 1, position 4, and position 7, and/or position 9; the GAG-binding peptide comprises a proline, arginine and/or isoleucine at position 1, position 4, position 7, and position 9; the GAG-binding peptide comprises a proline, arginine and/or isoleucine at position 1 and position 7; the GAG-binding peptide comprises a proline, arginine and/or isoleucine at position 1 and position 4 and position 9; the GAG-binding peptide comprises a proline, arginine and/or isoleucine at position 1 and position 9; and any combination therebetween. The GAG-binding peptide may comprise a proline at position 1, position 4, position 7, and position 9; the GAG-binding peptide may comprise an arginine at position 1, position 4, position 7, and position 9; the GAG-binding peptide may comprise an isoleucine at position 1, position 4, position 7, and position 9; the GAG-binding peptide may comprise a proline at position 1, and arginines at position 4, position 7, and position 9; the GAG-binding peptide may comprise a proline at position 1, arginines at position 4 and position 7, and an isoleucine at position 9; the GAG-binding peptide may comprise a proline at position 1, an arginine at position 4, and an isoleucine at position 9; or the GAG-binding peptide may comprise an arginine at position 4 and an proline at position 9. Any combinations of proline, arginine, and/or isoleucine at position 1, position 4, position 7, and/or position 9 is encompassed by the present disclosure.

In embodiments, the first and the second GAG-binding peptides independently comprise an amino acid sequence that is at least about 70% identical to one of SEQ ID NO: 1 to SEQ ID NO: 13.

In embodiments, the first and the second GAG-binding peptides independently comprise an amino acid sequence that is at least about 80% identical to one of SEQ ID NO: 1 to SEQ ID NO: 13.

In embodiments, the first and the second GAG-binding peptides independently comprise an amino acid sequence that is at least about 90% identical to one of SEQ ID NO: 1 to SEQ ID NO: 13.

In embodiments, the first and the second GAG-binding peptides independently comprise a charged amino acid at position 1, position 4, position 7, or position 9 with respect to any one of SEQ ID NO: 1 to SEQ ID NO: 13.

In embodiments, the first and the second GAG-binding peptides independently comprise a proline, arginine, and/or isoleucine at position 1, position 4, position 7, and/or position 9 with respect to any one of SEQ ID NO: 1 to SEQ ID NO: 13.

In embodiments, the first and the second GAG-binding peptides independently comprise at least 10 amino acids.

In embodiments, the first and the second GAG-binding peptides independently comprise 11 amino acids.

In embodiments, the first and the second GAG-binding peptides independently consist of 11 amino acids.

In embodiments, the GAG-binding peptide consists of the amino acid sequence of one of SEQ ID NO: 1 to SEQ ID NO: 13.

In embodiments, the first GAG-binding peptide comprises an amino acid sequence that is at least about 90% identical to SEQ ID NO: 1 and the second GAG-binding peptide comprises an amino acid sequence that is at least about 90% identical to SEQ ID NO: 2.

In embodiments, the first GAG-binding peptide comprises the amino acid sequence of SEQ ID NO: 1 and the second GAG-binding peptide comprises the amino acid sequence of SEQ ID NO: 2.

In embodiments, the first GAG-binding peptide consists of the amino acid sequence of SEQ ID NO: 1 and the second GAG-binding peptide consists of the amino acid sequence of SEQ ID NO: 2.

In embodiments, the first polypeptide consists of the first GAG-binding peptide and the second polypeptide consists of the second GAG-binding peptide.

In embodiments, the N-terminal of the first polypeptide is directly or indirectly linked to the first agent and/or the N-terminal of the second polypeptide is directly or indirectly linked to second first agent.

In embodiments, the C-terminal of the first polypeptide is directly or indirectly linked to the first agent and/or the C-terminal of the second polypeptide is directly or indirectly linked to second first agent.

In embodiments, the first agent is indirectly linked to the first polypeptide via a first linker and/or wherein the second agent is indirectly linked to the second polypeptide via a second linker. In some cases, the first linker and/or the second each comprise one or more atoms. The first linker and/or the second may each comprise a polymer of repeating units. The first linker and/or the second may each comprise a chain of amino acids.

In embodiments, the first agent is directly linked to the first polypeptide and/or the second agent is directly linked to the second polypeptide.

In embodiments, the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

In embodiments, the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet. In some cases, the first agent and/or the second agent comprises an antibody and/or comprises a fluorescent moiety.

In embodiments, the first agent and/or the second agent is harmful to mammalian cells and/or is toxic to a subject and/or the first agent and/or the second agent is susceptible to degradation when administered directly into the bloodstream of a subject.

In embodiments, the first compound and/or the second compound further comprises a fluorescent moiety. In embodiments, the first GAG-binding peptide and/or the second GAG-binding peptide also preferentially binds serglycin, perlecan, dermatan sulfate, keratan sulfate, and/or GPIIb/IIIa.

In embodiments, the isolated platelet further comprises at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

Notably, the loaded platelets of the present disclosure remain in a resting, fully functional platelet, rather than becoming activated by the loading process which would make the platelets pro-coagulant.

### Pharmaceutical Compositions

Loaded platelets of the present disclosure can be formulated into pharmaceutical compositions which enhance stability and effectiveness of the platelets, at least, once administered to a subject. Moreover, such pharmaceutical compositions enhance stability of the platelets prior to administration to the subject.

Yet another aspect of the present disclosure is a pharmaceutical composition comprising an isolated platelet of any herein disclosed aspect or embodiment and one or more pharmaceutically acceptable excipients.

In embodiments, the pharmaceutical composition further comprises a second isolated platelet comprising at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

In embodiments, the pharmaceutical composition further comprises a second isolated platelet comprising at least one copy of the first compound or further comprising a third isolated platelet comprising at least one copy of a second compound.

In embodiments, the pharmaceutical composition further comprises a second isolated platelet comprising at least one copy of the first compound and comprising a third isolated platelet comprising at least one copy of a second compound.

Pharmaceutical compositions comprise a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose (i.e., dextrose), lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents. Examples of suitable pharmaceutical excipients are described in Remington's Pharmaceutical Sciences 1447-1676 (Alfonso R. Gennaro eds., 19th ed. 1995), incorporated herein by reference.

The pharmaceutical composition disclosed herein may comprise a saline buffer (including, without limitation a NaCl solution, TBS, PBS, Ringer's solution, and the like).

In embodiments, the pharmaceutical compositions disclosed herein in the form suitable for sterile injection that is approximate isotonic to blood and that has a pH of between about 7.3 and 7.5 (i.e., the pH of blood).

In embodiments, the pharmaceutical composition disclosed herein is formulated in accordance with routine procedures as a pharmaceutical composition adapted for a mode of administration disclosed herein.

In an aspect, the present disclosure provides a use of any herein disclosed pharmaceutical composition for treating a disease or a disorder. In embodiments, the disease or disorder is a cancer.

In another aspect, the present disclosure provides a use of any herein disclosed isolated platelet or any herein disclosed pharmaceutical composition in the manufacture of a medicament for treating a disease or disorder. In embodiments, the disease or disorder is a cancer.

### Treatment Methods

As disclosed previously, platelets loaded with two or more compound comprising the two or more agents avoid a reduction in concentration of the agents (e.g., below an effective dose) which occurs when the agents are administered to the subject without loading into platelets. Additionally, platelets loaded with a compound comprising a harmful (e.g., toxic) agent avoids the unintended and undesirable cellular, tissue, and/or organ damage in the subject. Platelets naturally home to sites of injury, inflammation, and/or angiogenesis. The platelets of the present disclosure are selective loaded with two or more agents into distinct α-granule types, each of which have separate release profiles, thereby allowing release of different agents from platelets in a spatially- and temporally controlled fashion. Finally, release of the contents of loaded platelets of an alpha granule may be induced in response to contact with a release inducer which may be administered to a subject in a pharmaceutical composition and at a time that facilitates release of the agents as needed to promote a therapeutic response. Together, the loaded platelets help ensure that a therapeutically effective amounts of two or more agent is delivered to a target site and with fewer adverse effects.

Diseases and disorders characterized by tissue inflammation or tissue damage and characterized by platelets being a first responders, can all be treated according to the disclosed methods. These diseases and disorders include, but are not limited to, neoplasia, hematologic malignancies, rheumatoid arthritis, ulcerative colitis, stroke, ischemic heart disease, atherosclerosis, burns, and graft epithelization.

An advantage provided by the present invention is the prolonged half-life (in a subject's bloodstream) of an agent when loaded into a platelet relative to the agent directly administered to the bloodstream. The present invention slows the natural elimination of the agent is reduced significantly. Normally, an agent is eliminated from the circulation by renal filtration, enzymatic degradation, uptake by the reticulo-endothelial system (RES), and accumulation in non-targeted organs and tissues. However, in the present invention, the agent is protected within the platelet for the lifespan of the platelet (typically 4-7 days) or until delivered to the target site. In addition, the present invention limits exposure of the agent systemically by avoiding widespread distribution of the agent to non-target sites (e.g., tissues and organs). The benefits allow use of lower dosages of the agents (relative to administrations the agents that are not loaded into platelets). Such use of lower doses, at least, helps reduce unwanted side-effects and reduces economic costs.

Also, platelets useful in the present invention are loaded with a plurality of different agents; the different agents can be released from distinct alpha granule types in a spatially- and temporally controlled fashion. Accordingly, the present invention provides directed and controlled therapeutics to sites of injury (e.g., for treating chronic wounds), pathological inflammation (e.g., for treating injury to joints or lungs), and/or angiogenesis (e.g., for treating cancer).

In yet another aspect, the present disclosure provides a method for treating a disease or disorder in a subject in need thereof. The method comprises a step of administering to the subject a therapeutically effective amount of any herein disclosed pharmaceutical composition.

An aspect of the present disclosure is a method for treating a disease or disorder in a subject in need thereof. The method comprising a step of administering to the subject a therapeutically effective amount of any herein disclosed composition.

In embodiments of the above methods, the contents of the first alpha granule type is released at a target site before the contents of second alpha granule type is released.

In embodiments of the above methods, the method further comprises a step of administering to the subject a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase. In some cases, the second pharmaceutical composition promotes release of a first compound from a first alpha granule type and the third pharmaceutical composition promotes release of a second compound from a second alpha granule type. The second pharmaceutical composition and/or the third pharmaceutical composition may be administered after the pharmaceutical composition is administered. The pharmaceutical composition may be administered at least twice before the second pharmaceutical composition and/or the third pharmaceutical composition is administered.

In embodiments, the disease or disorder is a cancer. A cancer is generally disease caused by inappropriately high proliferation rate and/or inappropriately low rate of apoptosis.

In embodiments, the cancer is selected from acoustic neuroma; acute erythroleukemia; acute leukemia; acute lymphoblastic leukemia; acute lymphocytic leukemia; acute monocytic leukemia; acute myeloblastic leukemia; acute myelocytic leukemia; acute myelomonocytic leukemia; acute promyelocytic leukemia; adenocarcinoma; AIDS-related lymphoma; angiosarcoma; astrocytoma; basal cell carcinoma; B-cell lymphoma (including low grade/follicular non-Hodgkin's lymphoma); biliary tract cancer; bladder cancer; bone cancer; brain and central nervous system cancer; breast cancer; bronchogenic carcinoma; bulky disease non-Hodgkin's lymphoma; cancer of the digestive system; cancer of the head and neck; cancer of the peritoneum; cancer of the respiratory system; cancer of the urinary system; cervical cancer; chondrosarcoma; chordoma; choriocarcinoma; chronic leukemia; chronic lymphocytic leukemia; chronic myeloblastic leukemia; chronic myelocytic leukemia; colon and rectum cancer; connective tissue cancer; craniopharyngioma; cystadenocarcinoma; embryonal carcinoma; endometrial cancer; endotheliosarcoma; ependymoma; epithelial carcinoma; esophageal cancer; Ewing's tumor; eye cancer; fibrosarcoma; gastric cancer (including gastrointestinal cancer); glioblastoma; glioma; hairy cell leukemia; heavy chain disease; hemangioblastoma; hepatic carcinoma; hepatoma; high grade immunoblastic non-Hodgkin's lymphoma; high grade lymphoblastic non-Hodgkin's lymphoma; high grade small non-cleaved cell non-Hodgkin's lymphoma; Hodgkin's and non-Hodgkin's lymphoma; intermediate grade diffuse non-Hodgkin's lymphoma; intermediate grade/follicular non-Hodgkin's lymphoma; intra-epithelial neoplasm; kidney or renal cancer; larynx cancer; leiomyosarcoma; liposarcoma; liver cancer; lung cancer (e.g., small-cell lung cancer, non-small cell lung cancer, adenocarcinoma of the lung, and squamous carcinoma of the lung); lung carcinoma; lymphangioendotheliosarcoma; lymphangiosarcoma; lymphoma (Hodgkin's disease, non-Hodgkin's disease); mantle cell lymphoma; medullary carcinoma; medulloblastoma; Meigs' syndrome; melanoma; meningioma; mesothelioma; myeloma; myxosarcoma; neuroblastoma; bile duct carcinoma; oligodenroglioma; oral cavity cancer (lip, tongue, mouth, and pharynx); osteogenic sarcoma; ovarian cancer; pancreatic cancer; papillary adenocarcinomas; papillary carcinoma; pinealoma; polycythemia vera; post-transplant lymphoproliferative disorder (PTLD), as well as abnormal vascular proliferation associated with phakomatoses, edema (such as that associated with brain tumors); prostate cancer; rectal cancer; retinoblastoma; rhabdomyosarcoma; salivary gland carcinoma; sarcoma; schwannoma; sebaceous gland carcinoma; seminoma; skin cancer; small lymphocytic (SL) non-Hodgkin's lymphoma; squamous cell cancer; stomach cancer; sweat gland carcinoma; synovioma; testicular cancer; thyroid cancer; uterine or endometrial cancer; vulval cancer; Waldenstrom's Macroglobulinemia; and Wilm's tumor.

In embodiments, the disease or disorder the cancer is a proliferative disorder, e.g., a lymphoproliferative disease.

In embodiments, the disease of disorder is an injury, e.g., a burn, a spinal injury, an orthopedic injury, and wound.

In embodiments, the disease of disorder is hemophilia hemarthrosis.

In embodiments, the disease of disorder is inflammation, e.g., acute or chronic inflammation, including joint inflammation and lung inflammation.

In embodiments, the disease of disorder is a diabetic ulcer.

In embodiments, the disease of disorder is a side effect of an implant, graft, stent, or prosthesis.

In embodiments, a disease of disorder treated by methods of the present disclosure is caused by a defective gene. In these embodiments, the agent may be a recombinant polypeptide that replaces a missing or dysfunctional protein. Alternately, or additionally, the recombinant protein may be any one of the herein disclosed polypeptide-based agents, i.e., an antibody (or antigen-binding fragment thereof), a chemotherapeutic agent, an immune checkpoint inhibitor, a growth factor, a growth inhibitor, a protease/proteinase, a coagulation factor, an extracellular matrix protein, a hormone, an enzyme, a chemokine/chemoattractant, or a neurotrophin.

Some diseases caused by defects in genes may affect the synthesis of GAGs. As examples a defect in the Chondroitin Sulfate Proteoglycan 5 (CSPG5) on the long arm of Chromosome 3 can cause brain dysmorphogenesis and a defect in the DBQD1 gene causes micromelic dwarfism also called “Desbuquois dysplasia with hand anomalies”’ and the gene abnormality can affect the synthesis of GAGS in platelets.

Administration of a herein disclosed pharmaceutical composition results in delivery of the loaded platelets into the bloodstream via intravenous or intra-arterial injection or infusion. Alternately, a herein disclosed pharmaceutical composition is re administered directly to the site of active disease. Other routes of administration include, for example, subcutaneous, interperitoneally, intramuscular, or intradermal injections.

The dosage of a pharmaceutical composition comprising herein disclosed loaded platelets as well as the dosing schedule could depend on various parameters, including, but not limited to, the disease being treated, the subject's general health, and the administering physician's discretion.

The dosage can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

Generally, dosages of a pharmaceutical composition comprising a specific amount of the agent loaded into platelets will be in the range of those when the agent is administered without being loaded into platelets. In embodiments, the dosage of agent in a herein disclosed pharmaceutical composition will be lower than the dosage of the agent that is not loaded into platelets, since the present invention provides increased target specificity and resistance to degradation of the agent in the subject.

Any pharmaceutical composition comprising herein disclosed loaded platelets can be administered in a single daily dose, or the total daily dosage can be administered in divided doses of two, three or four times daily. Furthermore, any pharmaceutical composition comprising herein disclosed loaded platelets could be administered continuously rather than intermittently throughout the dosage regimen.

Loaded platelets may be infused into the patient repeatedly, e.g., once weekly, since the half-life of platelets is four to seven days.

### Recombinant Polypeptide Expression

The invention further provides fusion proteins comprising an amino acid sequence of a recombinant polypeptide agent coupled (directly or indirectly) to a polypeptide comprising a glycosaminoglycan (GAG)-binding peptide.

Recombinant polypeptides comprising a GAG-binding peptide may express as separate peptides and ligated together. Alternately, recombinant polypeptides comprising a GAG-binding peptide are expressed as a single fusion protein that includes the polypeptide agent operably linked to a GAG-binding peptide.

Recombinant polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to express the recombinant polypeptides. The precise host cell used is not critical to the invention. A recombinant polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the ATCC, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al., expression vehicles may be chosen from those provided, e.g., in Cloning Vectors: A Laboratory Manual (P. H. Pouwels et al., 1985, Supp. 1987).

Once the recombinant polypeptide of the invention is expressed, it may be isolated, concentrated, and/or purified

As an example, recombinant polypeptide may be isolated using affinity chromatography. In one example, an antibody raised against the recombinant polypeptide may be attached to a column and used to isolate the recombinant polypeptide. Lysis and fractionation of polypeptide-harboring cells prior to affinity chromatography may be performed by standard methods (see, e.g., Ausubel et al.,). Alternatively, the recombinant polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

### Combination Therapies

In embodiments, any herein disclosed pharmaceutical composition or method of treatment may further comprise an additional agent that is not linked to a glycosaminoglycan (GAG)-binding peptide and/or loaded into a platelet. In one example of a combination therapy, a pharmaceutical composition comprises loaded platelets and the additional agent. In another example of a combination therapy, a subject is administered a first pharmaceutical composition comprising loaded platelets and a second pharmaceutical composition comprising the additional agent. Combination therapies may also include a first pharmaceutical composition comprising loaded platelets and a first additional agent and a second pharmaceutical composition comprising a second additional agent; here, the first and second additional agents may be the same or may be different agents. Any agent disclosed herein may serve as an additional agent.

In embodiments combination therapy involving more than one pharmaceutical composition, a first pharmaceutical composition may be administered before a second pharmaceutical composition, a first pharmaceutical composition may be administered after a second pharmaceutical composition, or a first pharmaceutical composition may be administered simultaneous with a second pharmaceutical composition.

Additionally, a combination therapy may combine a pharmaceutical composition of the present disclosure with another treatment regimen. Other treatment regimen include radiotherapy, hormonal therapy, surgery, and cryosurgery. The treatment therapy may comprise any of the herein-described agent.

In embodiments, of a combination therapy, a chemotherapeutic agent is used in conjunction with a compound of the present disclosure. As examples, a combination therapy may comprise platelets loaded with one or both of a compound comprising a multikinase inhibitor (e.g., regorafenib) as agent, a compound comprising fumagillin as agent, and a chemotherapeutic agent; this combination may be used for treating pancreatic cancer, lung cancer, or colon cancer. A combination therapy may comprise platelets loaded with one or both of a compound comprising an EGFR inhibitor (e.g., Cetuximab) as agent, a compound comprising a multikinase inhibitor (e.g., regorafenib) as an active agent, and a chemotherapeutic agent; this may be used for treating lung cancer. A combination therapy may comprise platelets loaded with one or both or all three of a compound comprising an EGFR inhibitor (e.g., Cetuximab) as agent, a compound comprising a multikinase inhibitor (e.g., regorafenib) as agent, a compound comprising an ALK/ROS1/NTRK inhibitor (e.g., crizotinib) as agent, and a chemotherapeutic agent; this may be used for treating non-small cell lung cancer.

In additional embodiments, a combination therapy comprises platelets loaded with a VEGF inhibitor (e.g., Bevacizumab) and the drug Remdesivir; this may be used to treat Acute respiratory distress syndrome (ARDS), perhaps associated with COVID.

In embodiments of a combination therapy, a pharmaceutical composition may be administered before another treatment regimen, a pharmaceutical composition may be administered after another treatment regimen, or a pharmaceutical composition may be administered simultaneous with another treatment regimen.

### Manufacturing Methods

Another aspect of the present disclosure is a method for manufacturing a loaded platelet. The method comprising steps of: obtaining a platelet; contacting the platelet in vitro or ex vivo with any herein disclosed composition; and allowing contact between the platelet and the composition to progress until the first compound is internalized by a first alpha granule type of the platelet and the second compound is internalized by a second alpha granule type of the platelet, thereby producing a loaded platelet.

Yet another aspect of the present disclosure in a method for manufacturing a loaded platelet. The method comprising steps of: obtaining a platelet; contacting the platelet in vitro or ex vivo with a first compound comprising a first agent and a first polypeptide, wherein the first polypeptide comprises a first glycosaminoglycan (GAG)-binding peptide which is capable of binding a GAG in a first alpha granule type of a platelet; and contacting the platelet in vitro or ex vivo with a second compound comprising a second agent and a second polypeptide, wherein the second polypeptide comprises a second glycosaminoglycan (GAG)-binding peptide which is capable of binding a GAG in a second alpha granule type of the platelet.

In embodiments, the contacting the platelet with the first compound and the contacting the platelet with the second compound are contemporaneous.

In embodiments, the contacting the platelet with the first compound and the contacting the platelet with the second compound are sequential.

In embodiments, the method further comprises contacting the platelet in vitro or ex vivo with a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

Contact between a platelet and a composition (or a first compound and a second compound) may occur at 37° C. for at least about 15 minutes and/or until the first compound is internalized by a first alpha granule type of the platelet and the second compound is internalized by a second alpha granule type of the platelet. When an agent has significant systemic toxicity, the platelets are washed using a suitable buffer to prevent infusion of an agent that has not been loaded into a platelet.

### Kits

In an aspect, the present disclosure provides a kit for treating a disease or disorder. The kit comprising any herein disclosed isolated platelet and instructions for use.

In another aspect, the present disclosure provides a kit for treating a disease or disorder. The kit comprising any herein disclosed pharmaceutical composition and instructions for use.

In embodiments, the kit further comprises a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase.

In yet another aspect, the present disclosure provides a kit for manufacturing a loaded platelet. The kit comprising any herein disclosed composition and instructions for use. The invention provides kits for the treatment or prevention of diseases or disorders involving sites of injury, inflammation, or tumor angiogenesis. In one embodiment, the kit includes a therapeutic or prophylactic composition containing an effective amount of platelets loaded with two or more agent in unit dosage form. In some embodiments, the kit comprises a sterile container that contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition comprising an isolated platelet of the present disclosure is provided together with instructions for administering it to a subject having or at risk of developing a disease or disorder. The instructions may include information about the use of the pharmaceutical composition for the treatment or prevention of the disease or for delivery of an isolated platelet to a tissue in need thereof. In other embodiments, the instructions include at least one of the following: description of the agent; dosage schedule and administration for treatment or prevention of the disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

Any aspect or embodiment disclosed herein can be combined with any other aspect or embodiment as disclosed herein.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### DEFINITIONS

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The term “at least second” means a second, a third, a fourth, a fifth, a sixth, a seventh, an eighth, a ninth, a tenth, a twentieth, a thirtieth, a fourteenth, a fiftieth, a sixtieth, a seventieth, an eightieth, a ninetieth, a hundredth, or more and any iteration therebetween. The term “one or more” includes one, two, three, four, five, six, seven, eight, nine, ten, twenty, thirty, forty, fifty, sixty, seventy, eighty, ninety, one hundred, or more and any number therebetween.

The term “cargo” is meant a compound or agent that can be loaded into a platelet, e.g., an alpha granule of a platelet. Such loading occurs via a glycosaminoglycan (GAG)-binding peptide of a compound. In some embodiments, the term “agent” and “cargo” can be synonyms.

### REFERENCES

The contents of the following references are incorporated by reference in their entirety.
Gasic G J, Gasic T B, Galanti N, Johnson T, Murphy S. Platelet-tumor-cell interactions in mice. The role of platelets in the spread of malignant disease. International journal of cancer Journal international du cancer 1973; 11(3): 704-18.
Gasic G J, Gasic T B, Stewart C C. Antimetastatic effects associated with platelet reduction. Proceedings of the National Academy of Sciences of the United States of America 1968; 61(1): 46-52.
Gimbrone M A, Jr., Aster R H, Cotran R S, Corkery J, Jandl J H, Folkman J. Preservation of vascular integrity in organs perfused in vitro with a platelet-rich medium. Nature 1969; 222(5188): 33-6.
Rybak M E, Gimbrone M A, Jr., Davies P F, Handin R I. Interaction of platelet factor four with cultured vascular endothelial cells. Blood 1989; 73(6): 1534-9.
Hilgard P, Heller H, Schmidt C G. The influence of platelet aggregation inhibitors on metastasis formation in mice (3LL). Z Krebsforsch Klin Onkol Cancer Res Clin Oncol 1976; 86(3): 243-50.
Gasic G J. Role of plasma, platelets, and endothelial cells in tumor metastasis. Cancer metastasis reviews 1984; 3(2): 99-114.
Karpatkin S, Pearlstein E, Ambrogio C, Coller B S. Role of adhesive proteins in platelet tumor interaction in vitro and metastasis formation in vivo. The Journal of clinical investigation 1988; 81(4): 1012-9.
Karpatkin S, Ambrogio C, Pearlstein E. The role of tumor-induced platelet aggregation, platelet adhesion and adhesive proteins in tumor metastasis. Prog Clin Biol Res 1988; 283: 585-606.
Karpatkin S, Pearlstein E. Role of platelets in tumor cell metastases. Annals of internal medicine 1981; 95(5): 636-41.
Klement G L, Yip T T, Cassiola F, et al. Platelets actively sequester angiogenesis regulators. Blood 2009; 113(12): 2835-42.
Cervi D, Yip T T, Bhattacharya N, et al. Platelet-associated PF-4 as a biomarker of early tumor growth. Blood 2008; 111(3): 1201-7.
Klement G, Shai E, Varon D. The Role of Platelets in Angiogenesis. In: Michelson A, ed. Platelets. Third ed. Philadelphia, PA: Mosby Elsevier; 2013: 487-503.
Klement G L. Platelet Biomarkers in Tumor Growth. Current Proteomics 2011; 8(3): 169-80.
Peterson J E, Zurakowski D, Italiano J E, Jr., et al. Normal ranges of angiogenesis regulatory proteins in human platelets. American journal of hematology 2010; 85(7): 487-93.
Peterson J E, Zurakowski D, Italiano J E, Jr., et al. VEGF, PF4 and PDGF are elevated in platelets of colorectal cancer patients. Angiogenesis 2012; 15(2): 265-73.
Savore C, Zhang C, Muir C, et al. Perlecan knockdown in metastatic prostate cancer cells reduces heparin-binding growth factor responses in vitro and tumor growth in vivo. Clinical & experimental metastasis 2005; 22(5): 377-90.
Kareva I, Abou-Slaybi A, Dodd O, Dashevsky O, Klement G L. Normal Wound Healing and Tumor Angiogenesis as a Game of Competitive Inhibition. PloS one 2016; 11(12): e0166655.
Italiano J E, Jr., Richardson J L, Patel-Hett S, et al. Angiogenesis is regulated by a novel mechanism: pro- and antiangiogenic proteins are organized into separate platelet alpha granules and differentially released. Blood 2008; 111(3): 1227-33.
Ma L, Hollenberg M D, Wallace J L. Thrombin-induced platelet endostatin release is blocked by a proteinase activated receptor-4 (PAR4) antagonist. Br J Pharmacol 2001; 134(4): 701-4.
Ma L, Perini R, McKnight W, et al. Proteinase-activated receptors 1 and 4 counter-regulate endostatin and VEGF release from human platelets. Proceedings of the National Academy of Sciences of the United States of America 2005; 102(1): 216-20.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EXAMPLES

### Example 1: Glycosaminoglycan (GAG)-Binding Peptides Sequester Attached Cargos into Alpha Granules of Platelets

In this example, the ability of illustrative glycosaminoglycan (GAG)-binding peptides to direct loading of a cargo into alpha granules of platelets was determined.

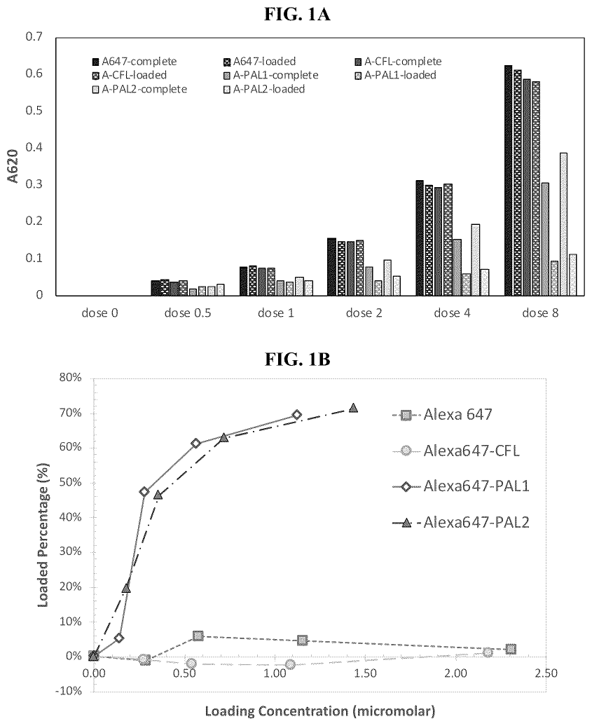

Alexa647-labeled GAG-binding peptides, identified in FIG. 1A and FIG. 1B as PAL1 and PAL2 and an Alexa647-labeled control peptide (a charge-free ligand (CFL) which served as a negative control), were tested for their binding affinity for glycosaminoglycans, such as chondroitin sulfate, and their abilities to enter platelets. PAL1 had an amino acid sequence of SEQ ID NO: 1, PAL2 had an amino acid sequence of SEQ ID NO: 2, CFL had an amino acid sequence of SEQ ID NO: 14.

A dose response curve of Alexa647-labeled peptides (or Alexa647 alone as a negative control) is shown in FIG. 1A. Alexa647-labeled peptides or Alexa647 alone were co-incubated with isolated platelets at 37° C. for one hour to allow for platelet loading. The respective platelet-loading ability was indicated by a decrease in fluorescence in supernatant following the incubation. For controls, identical experiments were performed without the incubation period (noted as “complete” in the figure). Platelets following co-incubation were then centrifuged at 800 g for 10-minutes to separate platelets from supernatant (noted as “loaded” in the figure).

As shown in FIG. 1A, there was a decrease in absorbance for PAL1 and PAL2 between the complete measurements and the loaded measurements. This reduction in absorbance from the supernatant indicates that these peptides had become sequestered from the supernatant and loaded into platelets. In contrast, absorbances of the Alexa647-labeled CFL conditions did not change after co-incubation with platelets; thus, the CFL peptides remained in the supernatant and were not loaded into platelets.

FIG. 1B represents the data in FIG. 1A normalized for each peptide experiment, i.e., normalization of a loaded condition to its complete condition. FIG. 1B shows that the illustrative GAG-binding peptides, PAL1 and PAL2, facilitates loading of an attached cargo into platelets whereas cargos attached to a charge-free ligand are unable to direct loading of the cargo into platelets.

To confirm that the Alexa647-labeled GAG-binding peptides were loaded into alpha granules of platelets, confocal microscopy was used. The platelets that were centrifuged in the experiments of FIG. 1A and FIG. 1B, were fixed in 2% paraformaldehyde and settled onto glass coverslips. After permeabilization, immunofluorescence staining was performed against PF4, which is a marker for alpha granules of platelets. Platelets were stained with Alexa568-secondary antibody. Images were collected through a Nikon-A1 laser-scanning microscope equipped with a 60× oil objective lens.

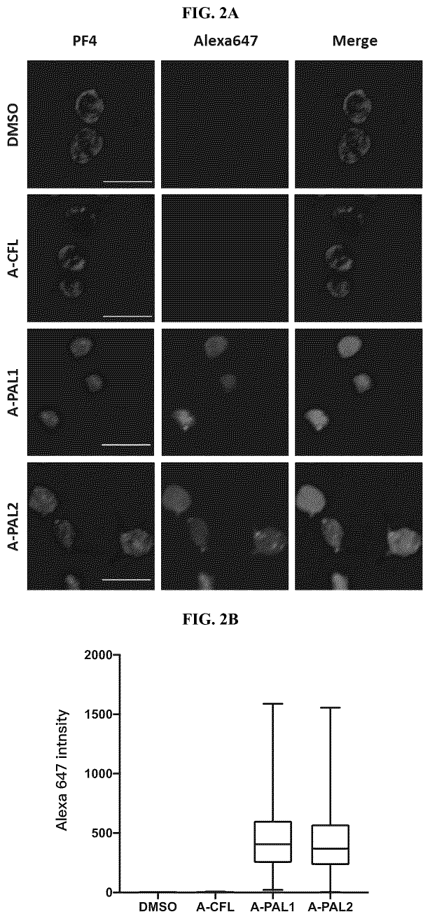

FIG. 2A are representative images with PF4 staining shown in red (left column) and the Alexa647 signal (from the free Alexa647, Alexa647-labeled GAG-binding peptide, or Alexa647-labeled CFL; middle column) shown in purple. Images were only adjusted for brightness and contrast for display. n>5 images were acquired for each experiment and regions of interest (ROIs) were selected based on PF4 intensity.

The merged images (right column) demonstrate colocalization of the alpha granule marker PF4 and the Alexa647 signal only when Alexa647 was the cargo for a GAG-binding peptide. Co-localization was not observed for free Alexa647 or when Alexa647 was the cargo of the CFL.

The Alexa647 intensities for each ROI were measured using ImageJ and plotted in box and whisker graph using Prism 8. FIG. 2B shows that the illustrative GAG-binding peptides, PAL1 and PAL2, facilitates loading of an attached cargo into alpha granules of platelets, whereas cargos attached to a charge-free ligand do not load into platelets, let alone into alpha granules of platelets.

These data demonstrate that the GAG-binding peptides of the present disclosure facilitate loading of any attached cargo into alpha granules of platelets.

### Example 2: Glycosaminoglycan (GAG)-Binding Peptides Bind Glycosaminoglycans with High Affinities

In this example, the binding affinities of illustrative glycosaminoglycan (GAG)-binding peptides to various glycosaminoglycans were determined.

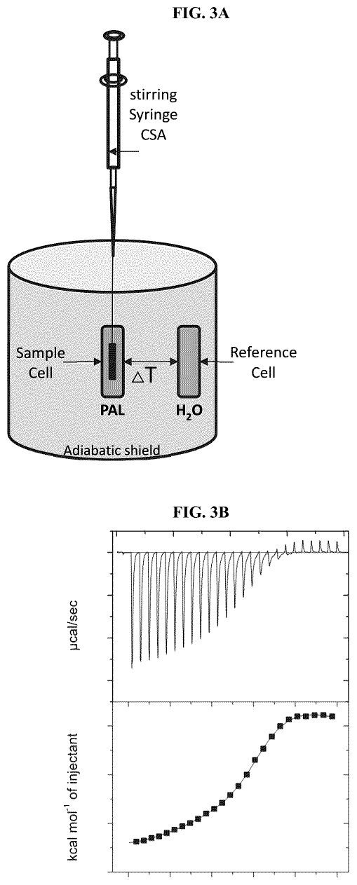

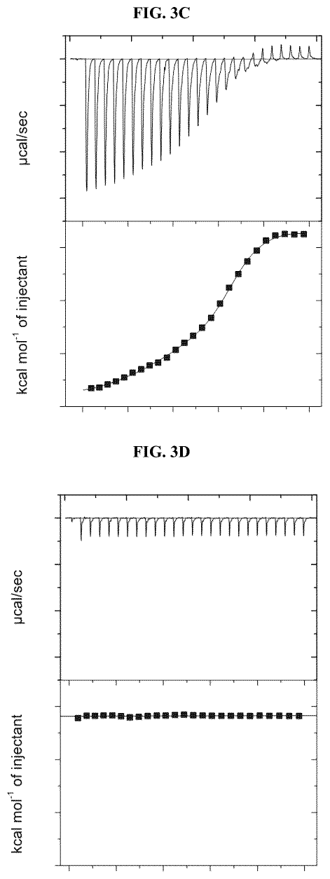

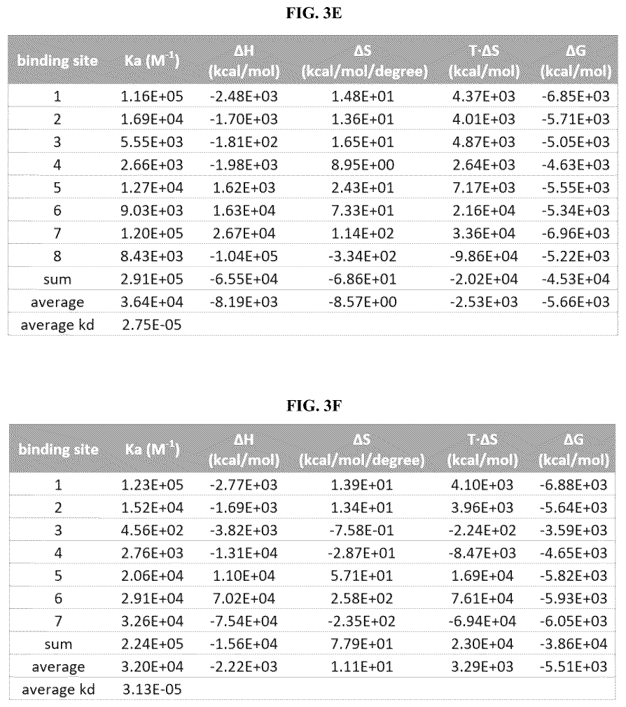

FIG. 3A is a schematic depicting the isothermal titration calorimetry (ITC) experiments performed in this example. Here, chondroitin sulfate A (CSA) was used to test affinities of illustrative GAG-binding peptides for glycosaminoglycan. 3 mM CSA was loaded into a syringe and CSA was titrated into the sample cell withholding a 0.25 mM solution of GAG-binding peptide or a charge-free ligand (CFL), which served as a negative control. Temperature was set at 22° C., the buffer was 5 mM Tris-HCl (pH 7.35), and 1% DMSO. Twenty-six injections of CSA were made, the first had a volume of 0.1 □1 and the subsequent twenty-five had volumes of 1.5 □1 each. In these experiments, the illustrative GAG-binding peptides were PAL1 and PAL2, respectively, having amino acid sequences of SEQ ID NO: 1 and SEQ ID NO: 2, and the CFL had an amino acid sequence of SEQ ID NO: 14.

FIG. 3B to FIG. 3D show graphical representations of ITC dissociation kinetics for CSA titrated into cells withholding PAL1 (FIG. 3B), PAL2 (FIG. 3C), and CFL (FIG. 3D).

The data obtained during the experiments of FIG. 3B and FIG. 3C were used to determine dissociation constants for the CSA and GAG-binding peptide interactions; these were determined through titration curve fitting using sequential binding model. These data are shown in FIG. 3E (for PAL1) and FIG. 3F (for PAL2). These data show that the two illustrative GAG-binding peptides have high affinity for the glycosaminogly can chondroitin sulfate A.

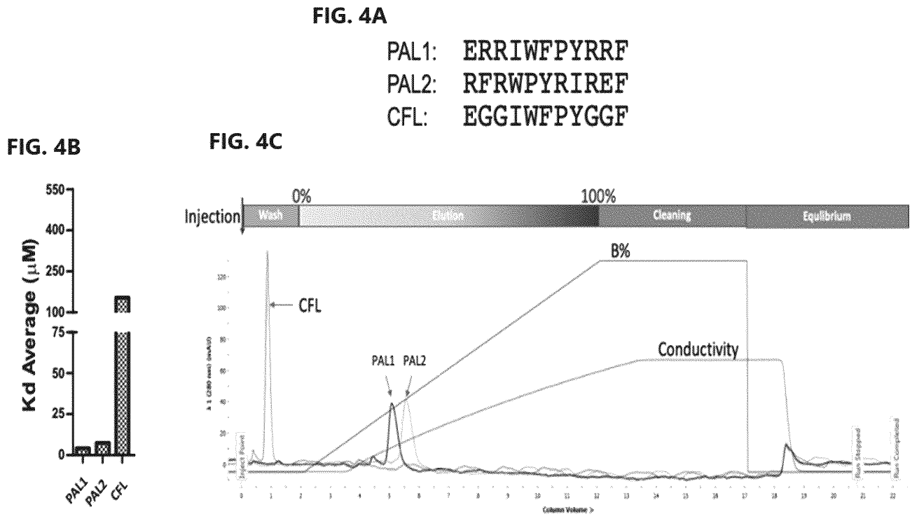

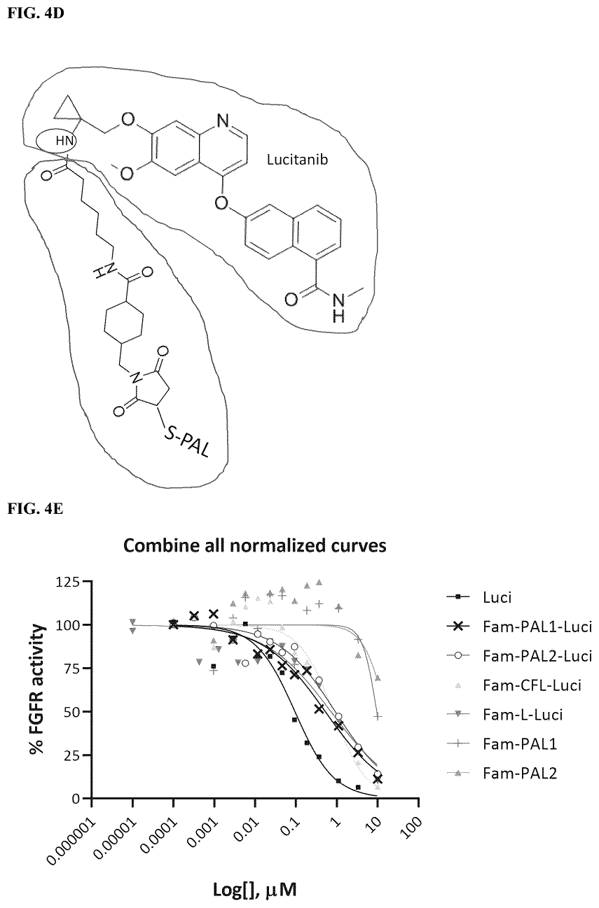

Additionally, the binding affinities for the two illustrative GAG-binding peptides, PAL1 and PAL2, to Heparan Sulfate (HS) and Chondroitin Sulfate (CSA) was determined using affinity chromatography. As shown in FIG. 4B, PAL1 is shown to bind CSA tighter than PAL2. The dissociation constants were measured using ITC, the higher it is, the looser the binding is. In FIG. 4C, PAL2 is shown binds HS tighter than PAL1. The binding was measured by the elution volume on a Hi-Trap Heparin column attached to a FPLC system. The later the peak occurs, the tighter the binding is. In FIG. 4A, the peptide amino acid sequences including the control peptide (CFL) and the two PALs (PAL1 and PAL2) are shown.

These data demonstrate that the two PAL sequences of the present disclosure have high affinity for glycosaminoglycans which are present in alpha granules of platelets and that they show different binding preference for two major glycosaminoglycans, with PAL1 binding tighter to CSA than PAL1 and PAL2 binding tighter to HS than PAL1.

### Example 3: Compounds Comprising a Glycosaminoglycan (GAG)-Binding Peptide and an Agent Load into Alpha Granules of Platelets

In this example, the ability of illustrative compounds comprising a glycosaminoglycan (GAG)-binding peptide and an agent to load into alpha granules of platelets was determined.

Two illustrative compounds of the present disclosure and two control compounds were constructed. The illustrative compounds included an agent (e.g., mNeonGreen) indirectly linked (via a nine amino acid linker) to a glycosaminoglycan (GAG)-binding peptide. In these experiments, the illustrative GAG-binding peptides were PAL1 and PAL2, respectively, having amino acid sequences of SEQ ID NO: 1 and SEQ ID NO: 2. The negative control compound included a charge-free ligand (CFL), having an amino acid sequence of SEQ ID NO: 14, indirectly linked (via the nine amino acid linker) to mNeonGreen. The positive control compound included PF4 (a natural platelet factor) indirectly linked (via the nine amino acid linker) to mNeonGreen. Prior to use, the compounds also included a His-tag for purification purposes, as well as a TEV-protease cleavage site, which facilitated removal of the His-tag. The compounds were identified as mCFL (for mNeon-L9-CFL), mPAL1 (for mNeon-L9-PAL1), mPAL2 (for mNeon-L9-PAL2), and PF4m (for PF4-L9-mNeon).

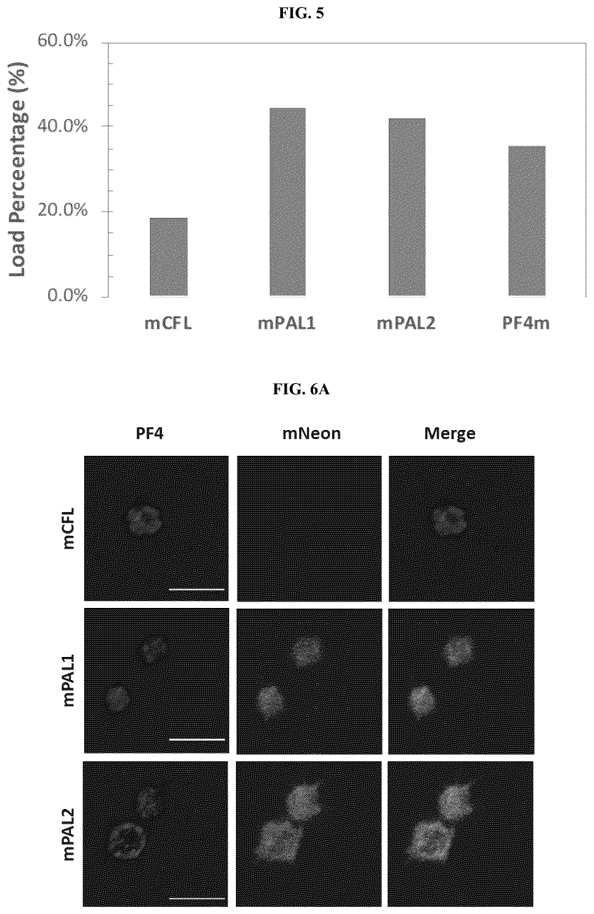

Platelets were co-incubated at 37° C. for an hour with one of the four compounds. After the incubation period, platelets were centrifuged at 800 g for 10-minutes. Then, the fluorescence absorbances of the “loaded” supernatants (at 505 nm) were measured and compared with the “complete” loading control, which was supernatants for each condition in which platelets were mixed with a compound and then immediately centrifuged, without an incubation period. The data were further normalized and the loading percentage for each group of experiments were plotted as shown in FIG. 5.

FIG. 5 shows that the two illustrative compounds had greater loading ability into platelets than the negative control and a slightly greater loading ability than the positive control PF4.

To confirm that the compounds comprising a GAG-binding peptide were loaded into alpha granules of platelets, confocal microscopy was used. The platelets that were centrifuged in the experiment of FIG. 5, were fixed in 2% paraformaldehyde and settled onto glass coverslips. After permeabilization, immunofluorescence staining was performed against PF4, which is a marker for alpha granules of platelets. Platelets were stained with Alexa568-secondary antibody. Images were collected through a Nikon-A1 laser-scanning microscope equipped with a 60× oil objective lens.

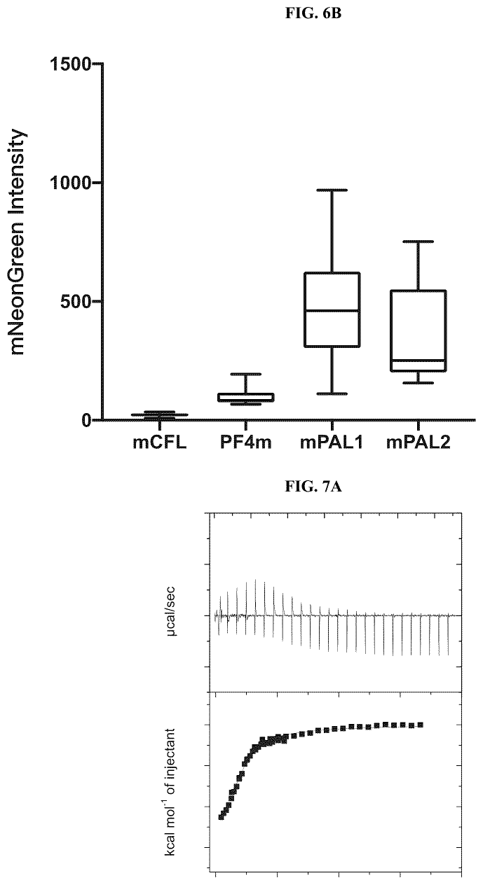

FIG. 6A are representative images with PF4 staining shown in red (left column) and the mNeon signal labeled green (middle column). Images were only adjusted for brightness and contrast for display. n>5 images were acquired for each experiment and regions of interest (ROIs) were selected based on PF4 intensity.

The merged images (right column) demonstrate colocalization of the alpha granule marker PF4 and the mNeon signal for the two illustrative compounds that comprise a GAG-binding peptide. Colocalization was not observed for the compound comprising the CFL.

The mNeon intensities for each ROI were measured using ImageJ and plotted in box and whisker graph using Prism 8. FIG. 6B. shows that the illustrative compounds comprising the GAG-binding peptides load into alpha granules of platelets whereas compounds comprising a charge-free ligand do not load into platelets, let alone into alpha granules of platelets.

These data demonstrate that compounds of the present disclosure which comprise a GAG-binding peptide and an agent load into alpha granules of platelets.

### Example 4: Compounds Comprising a Glycosaminoglycan (GAG)-Binding Peptide and an Agent Bind Glycosaminoglycans with High Affinities

In this example, the binding affinities of illustrative compounds of the present disclosure (which comprise a glycosaminoglycan (GAG)-binding peptide and an agent) to various glycosaminoglycans were determined.

Isothermal titration calorimetry (ITC) experiments as depicted in FIG. 3A and as described in Example 2 were performed in this example, yet with illustrative compounds of the present disclosure, with a negative control compound. Like the experiments of Example 2, here, the titration buffer was 5 mM Tris-HCl (pH 7.35) and the temperature set at 22° C.; however, unlike the experiments of Example 2, the buffer lacked DMSO.

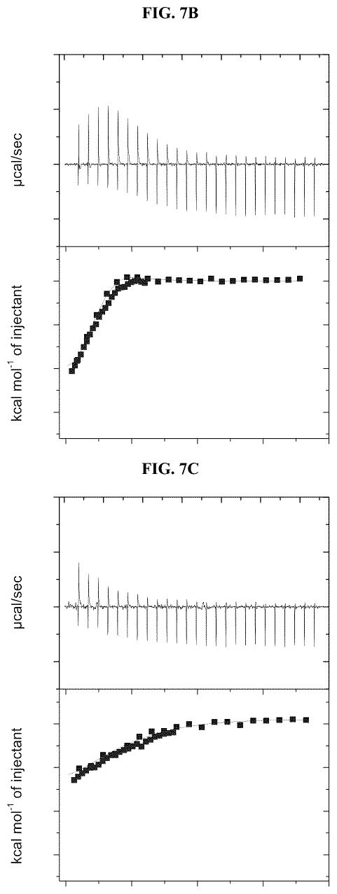

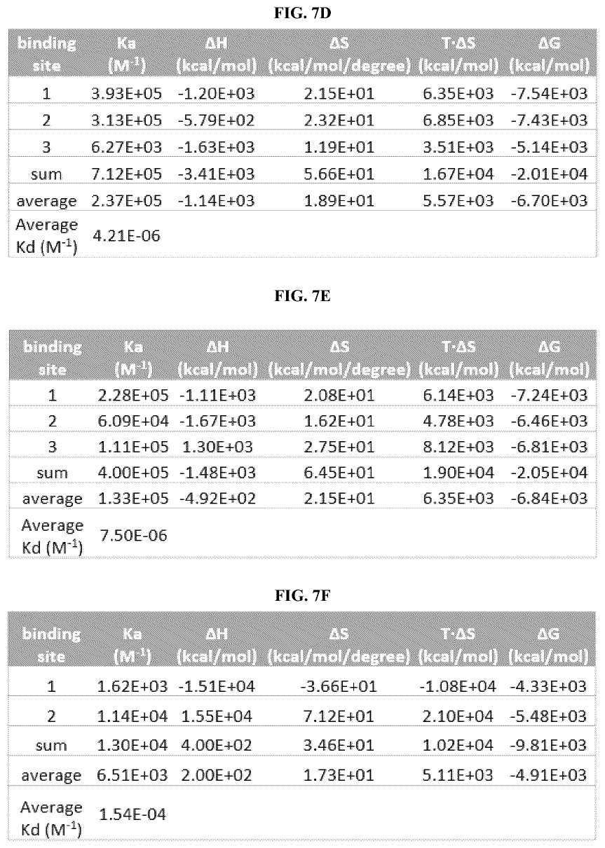

FIG. 7A to FIG. 7C show graphical representations of ITC dissociation kinetics for CSA titrated into cells withholding the illustrative compound comprising PAL1 (FIG. 7A), the illustrative compound comprising PAL2 (FIG. 7B), and the negative control compound comprising CFL (FIG. 7C). These compounds comprised mNeonGreen as its agent.

The data obtained during the experiments of FIG. 7B to FIG. 7C were used to determine dissociation constants for the CSA and compound interactions; these were determined through titration curve fitting using sequential binding model. These data are shown in FIG. 7C (for the illustrative compound comprising PAL1), FIG. 7D (for the illustrative compound comprising PAL2), and FIG. 7E (for the negative control compound comprising CFL). These data show that the two illustrative GAG-binding peptides have high affinity for the glycosaminoglycan chondroitin sulfate A.

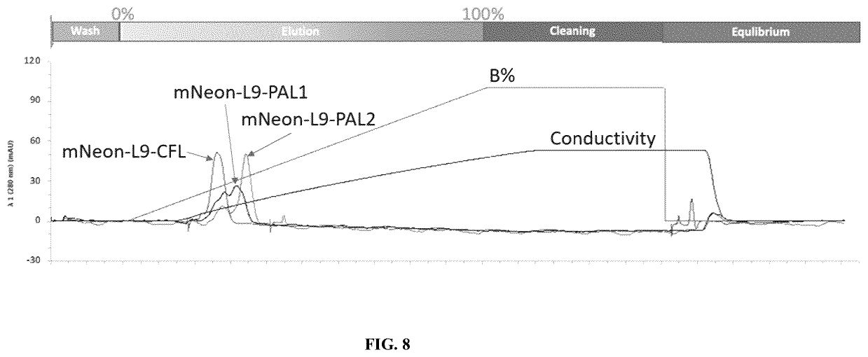

Additionally, the binding affinities for the two illustrative GAG-binding peptide containing compounds and the CFL to heparan sulfate (HS) was determined using affinity chromatography. As shown in FIG. 8, compounds comprising either GAG-binding peptide bind HS with high affinity. Notably, the relative binding affinities of the two illustrative GAG-binding peptides to HS were similar to that observed in prior experiments in that mPAL2 binds HS tighter than mPAL1 as PAL2 binds HS tighter than PAL1. Compounds comprising the control peptide (mCFL) has some residual binding ability and retained on the HS column which was eluted at a relatively low concentration of salt, perhaps due to charged character of the compound's agent (e.g., mNeonGreen).

These data demonstrate that the illustrative compounds of the present disclosure comprising glycosaminoglycan (GAG)-binding peptides and an agent have high affinity for glycosaminoglycans, which are in alpha granules of platelets.

### Example 5: Identification of Sequence Specificity Important for a Glycosaminoglycan (GAG)-Binding Peptide's Ability to Bind Glycosaminoglycans

In this example, the binding affinities of additional illustrative compounds comprising glycosaminoglycan (GAG)-binding peptides to a various glycosaminoglycan were determined. More specifically, alanine-scanning mutagenesis of the GAG-binding peptide (of SEQ ID NO: 1) produced additional illustrative GAG-binding peptides that differed by one amino acid, which were then indirectly linked to an agent (e.g., mNeonGreen), as described in Example 3.

Isothermal titration calorimetry (ITC) experiments as depicted in FIG. 3A and as described in Example 4 were performed in this example, yet with additional illustrative compounds of the present disclosure.

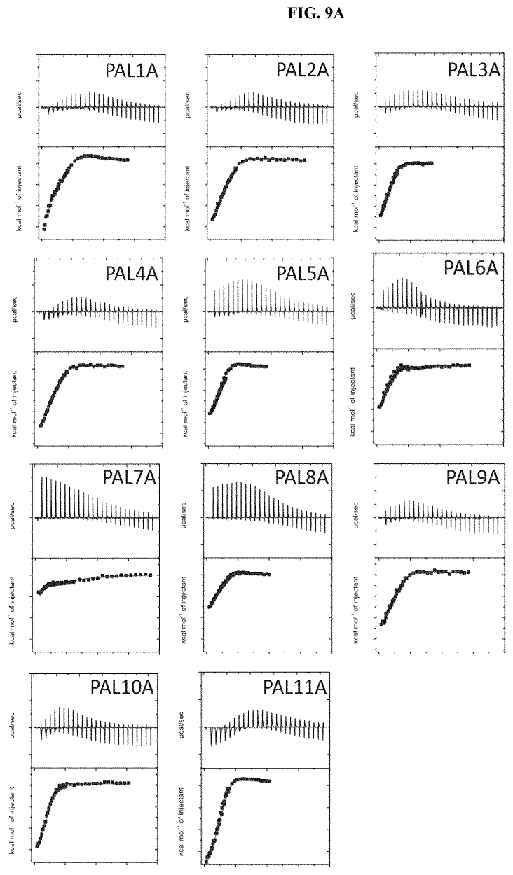

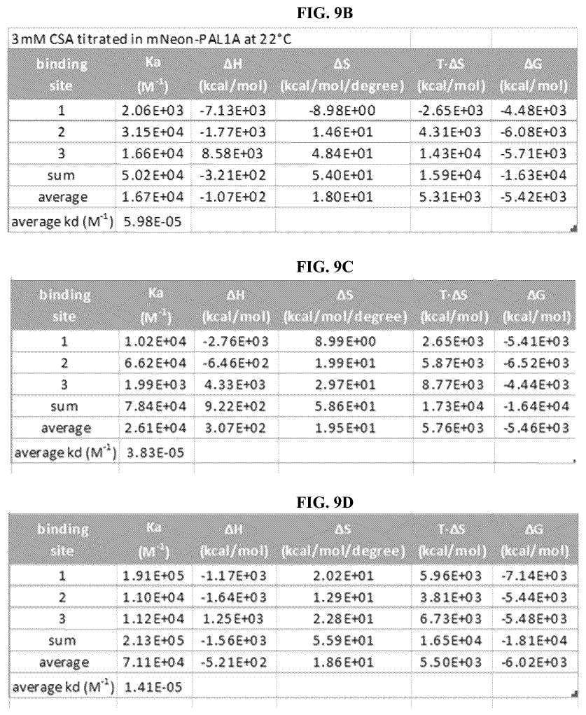

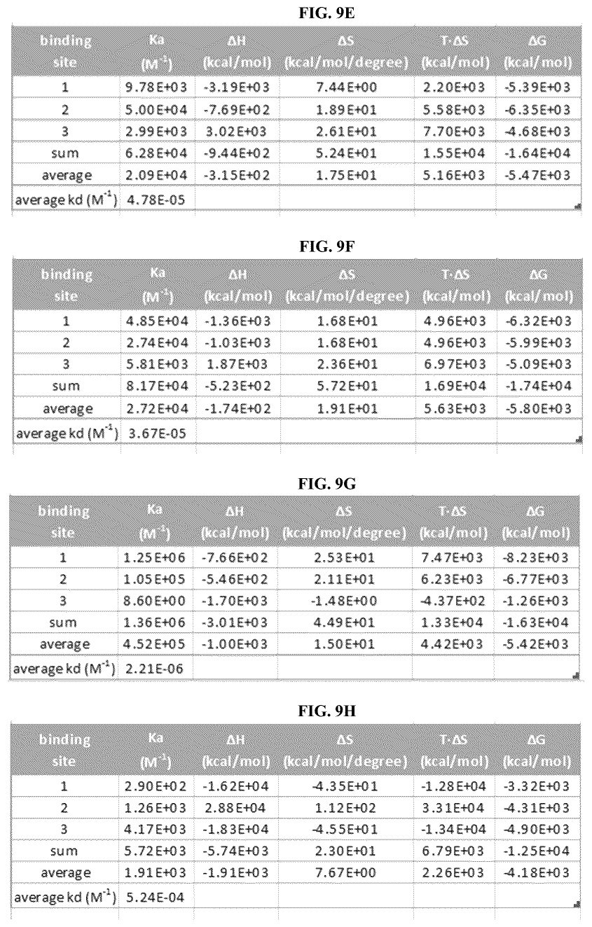

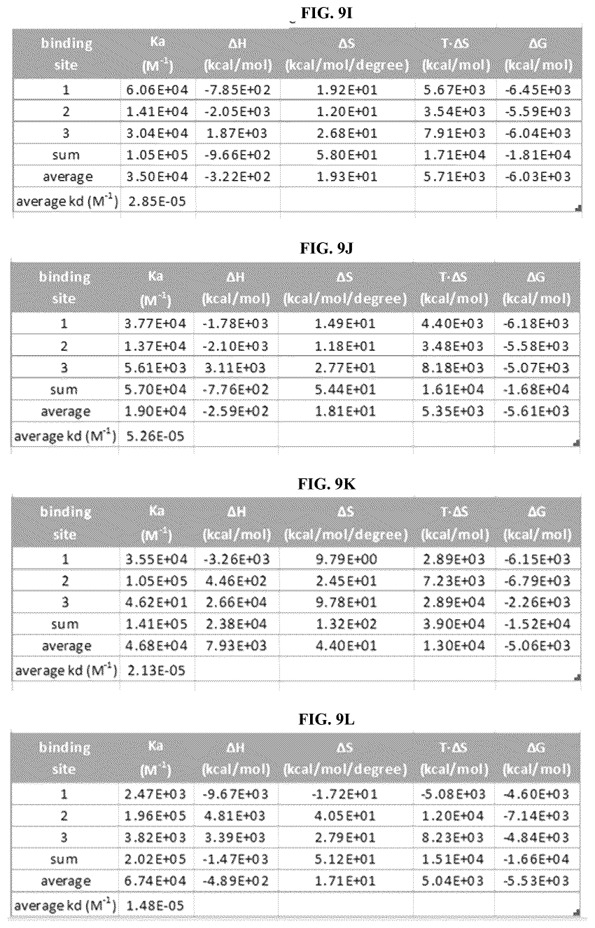

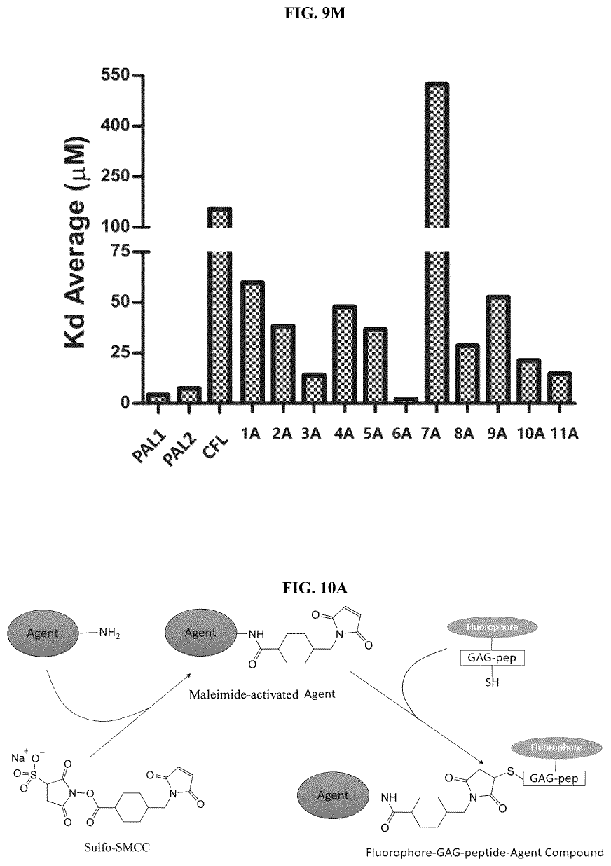

In FIG. 9A, the compounds are identified as PAL1A to PAL11A. These illustrative compounds have GAG-binding peptides having amino acid sequences of SEQ ID NO: 3 to SEQ ID NO: 13. In particular, the GAG-binding peptide of PAL1A differed from SEQ ID NO: 1 by having an alanine at position 1; the GAG-binding peptide of PAL2A differed from SEQ ID NO: 1 by having an alanine at position 2; and the GAG-binding peptide of PAL3A differed from SEQ ID NO: 1 by having an alanine at position 3.

FIG. 9A shows graphical representations of ITC dissociation kinetics for CSA titrated into cells withholding one of the illustrative compounds identified as PAL1A to PAL11A. As seen in the respective ITC curves generated by CSA titration into sample cells containing each listed compound, both charges and sequences are important in interacting with chondroitin sulfate A.

The data obtained during the experiments of FIG. 9A were used to determine dissociation constants for the CSA and additional illustrative compound interactions; these were determined through titration curve fitting using sequential binding model. These data are shown in FIG. 9B to FIG. 9L (respectively for PAL1A to PAL11A). These data show that the additional illustrative compounds have variable affinity for the glycosaminoglycan chondroitin sulfate A.

FIG. 9M is a graph depicting the average dissociation constants for the illustrative compounds and the control compound. This graph shows various magnitudes of CSA-binding affinities among the compounds. In the graph, to data identified as “1A” represents the “PAL1A” compound, to data identified as “2A” represents the “PAL2A” compound, and so forth.

Notably, those illustrative compounds having an alanine at its position 1, 4, 7, or 9 had the lowest, poorest affinity. Thereby demonstrating improvements in binding ability when a GAG-binding peptide has a proline, arginine, and/or isoleucine at those positions.

Critical amino acids such as proline, arginine, and isoleucine in positions affect the affinity of the binding. Interestingly, these amino acids include the positively charged arginine as expected and also non-charged proline and isoleucine that may contribute through maintain special conformation.

These data demonstrate that the additional compounds having GAG-binding peptides that differed in the position of a charged amino acid have variable affinity for glycosaminoglycans. And, critical residues (positions 1, 4, 7, and 9 with respect to SEQ ID NO: 1) and specific amino acids (such as proline, arginine, and isoleucine) affect the binding affinity of a GAG-binding peptide to a glycosaminoglycan, e.g., in an alpha granule of a platelet.

### Example 6: Illustrative Methods for Conjugating a Glycosaminoglycan (GAG)-Binding Peptide to an Agent when Forming a Compound of the Present Disclosure

In this example, an agent is conjugated to a glycosaminoglycan (GAG)-binding peptide to form an illustrative compound of the present disclosure.

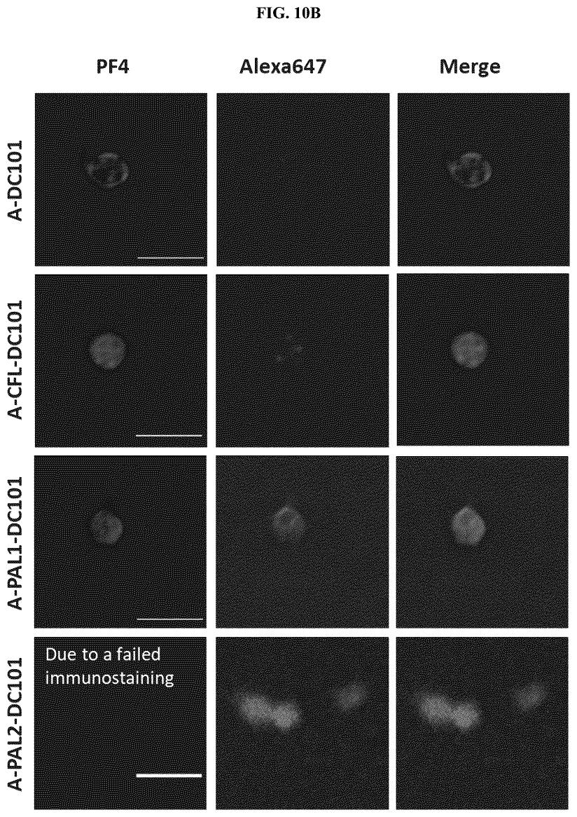

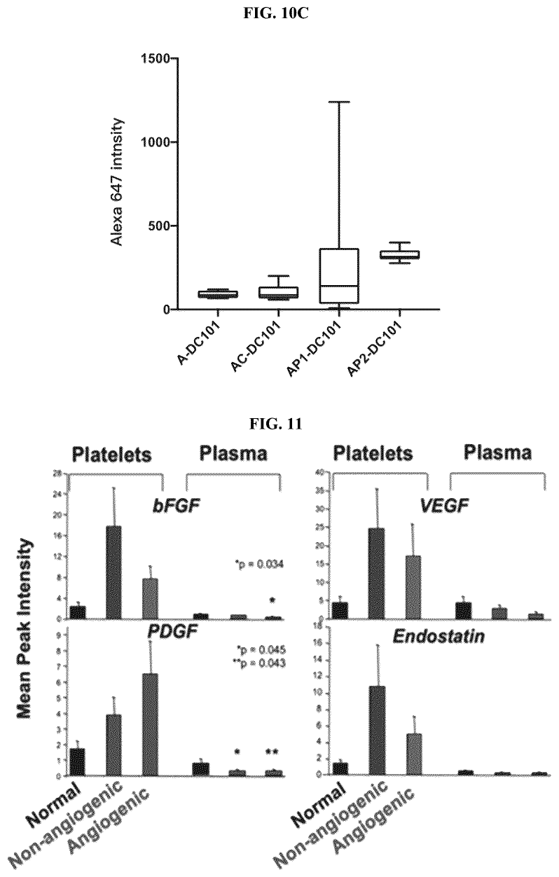

As shown in FIG. 10A, an agent is conjugated to a GAG-binding peptide using a maleimide reaction, thereby forming a compound of the present disclosure. Other conjugation reactions known in the art, e.g., succinimidyl ester reaction or an enzymatic reaction, may be used. In FIG. 10A, the GAG-binding peptide (shown in FIG. 10A as “GAG-pep”) comprises a fluorescent moiety; in certain embodiments of the present disclosure, a fluorescent moiety is not included in a compound.

To further demonstrate the ability of a compound of the present disclosure to load its cargo into platelets (as described in the above examples), here, an illustrative compound comprising a GAG-binding peptide and a therapeutic antibody (DC101, a VEGFR2 inhibitor) was produced. Using similar methods, agents other than antibodies can be used to produce a compound of the present disclosure. As examples, the agent may be a chemotherapeutic agent, a cytotoxic compound, a small molecule, a fluorescent moiety, radioactive element, or a factor that inhibits cellular proliferation, angiogenesis, inflammation, immunity, or another physiological process mediated by or associated with a platelet.

The ability of the illustrative compound (comprising an antibody as agent) and further comprising a fluorescent moiety to be loaded into alpha granules of platelets was determined.

Four compounds were prepared: an Alexa647-labled DC101 as a negative control (identified FIG. 10B as A-DC101), an Alexa647-labled compound comprising the charge-free ligand (CFL) of SEQ ID NO: 14 and the DC101 antibody (identified FIG. 10B as A-CLF-DC101), an Alexa647-labeled compound comprising the GAG-binding peptide of SEQ ID NO: 1 and the DC101 antibody (identified FIG. 10B as A-PAL1-DC101), and Alexa647-labeled compound comprising the GAG-binding peptide of SEQ ID NO: 2 and the DC101 antibody (identified FIG. 10B as A-PAL2-DC101).

Platelets were co-incubated with each compound for one hour at 37° C. The platelets were then centrifuged for 10-minutes at 800 g, fixed in 2% paraformaldehyde, and settled onto glass coverslips. After permeabilization, immunofluorescence staining was performed against PF4 in platelets and further stained with Alexa568-secondary antibody. The images were collected through a Nikon-A1 laser-scanning microscope equipped with a 60× oil objective lens.

In the representative images of FIG. 10B, PF4 staining was displayed in red (left column) and the Alexa647 signal was shown in purple (middle column). Images were only adjusted for brightness and contrast for display. n>5 images were acquired for each experiment and regions of interest (ROIs) were selected based on PF4 intensity.

The merged images (right column) demonstrate colocalization of the alpha granule marker PF4 and the Alexa647 signal only when Alexa647 was associated with a GAG-binding peptide, but not when Alexa647 was associated with the CFL or with the DC101 antibody alone. Unfortunately, the PF4 immunostaining reaction failed for the platelets co-incubated with the A-PAL2-DC101 compound. Therefore, ROIs were selected based on Alexa647 intensity for this group.

The Alexa647 intensities for each ROI were measured using ImageJ and plotted in box and whisker graph using Prism 8. As shown in FIG. 10C, the two illustrative compounds of the present disclosure load into alpha granules of platelets whereas the compound comprising a charge-free ligand or the compound comprising an antibody (without a GAG-binding peptide) do not load into platelets, let alone into alpha granules of platelets.

These data demonstrate that compounds of the present disclosure comprising a GAG-binding peptide and an agent load into alpha granules of platelets.

### Example 7: Disease-Relevant Proteins are Taken into Platelet Alpha Granules Actively and Against a Concentration Gradient

In this example, protein sequestration by platelets was analyzed and it was discovered that disease-relevant proteins are taken into platelet alpha granules actively and against a concentration gradient, whereas tumor irrelevant proteins such as albumin are not.

The role of platelets in thrombosis, wound healing and atherosclerosis is well established, but the role of platelets in tumor growth and metastasis is less clear. Publications dating back into the 1960's suggest that platelets aggregate in tumors, support tumor and endothelial cell growth, enhance tumor metastasis, and sequester cancer-specific proteins.

Surface Enhanced Laser Desorption/Ionization—Time of Flight Mass Spectrometry (SELDI-ToF MS) was used in a murine model of human liposarcoma to evaluate platelet and plasma protein profiles. It was found that platelets from mice bearing nonangiogenic (dormant) and angiogenic (rapidly growing) human tumor xenografts had much higher levels of tumor specific proteins (i.e., VEGF, bFGF, PDGF) than normal sham-operated mice (See, FIG. 11).

FIG. 11 are diagrams showing that platelet levels of bFGF, VEGF, PDGF, and endostatin change just prior to tumor escape from dormancy with the balance being towards stimulators of tumor growth. Platelets from mice bearing dormant (blue, middle columns), or angiogenic (red, right columns) xenografts of human liposarcoma were analyzed using Surface Enhanced Laser Desorption/Ionization (SELDI) Time of Flight (ToF) Mass Spectrometry (MS). The mean MS peak intensities from tumor-bearing mice were compared with those from platelets of healthy sham-operated mice (black, left columns) As evident, dormant and angiogenic tumors have significant elevation of cancer related bFGF, VEGF and PDGF, but endostatin, a cancer inhibitor, is decreased with cancer progression. On the other hand, escape from dormancy (angiogenic growth, in red) was associated with a decrease in inhibitors (endostatin).

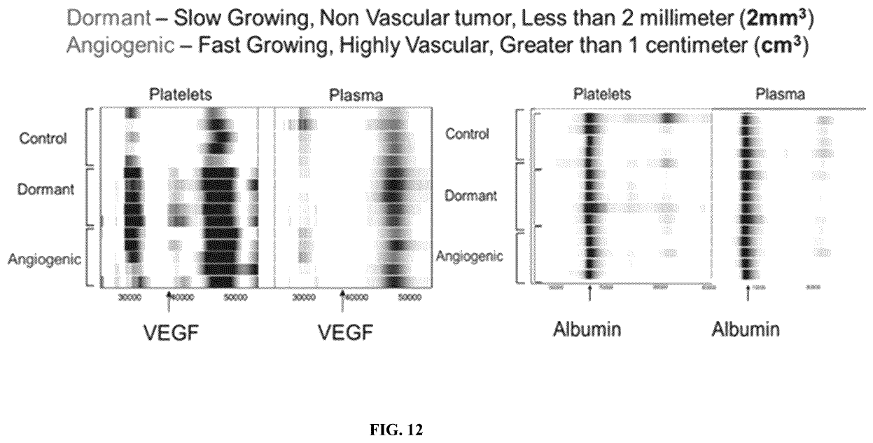

Furthermore, it was found that platelets actively sequestered select cancer-specific proteins, whereas non-specific proteins such as albumin were not sequestered. (See, FIG. 12)

FIG. 12 are MS expression maps showing that platelets actively sequester cancer specific proteins, and do not actively sequester non-specific proteins such as albumin. Platelets from mice bearing dormant (labeled blue, middle row), and mice bearing angiogenic (red, bottom rows) xenografts of human liposarcoma were analyzed using SELDI ToF MS. As shown, in the left-hand panel, Vascular Endothelial Growth Factor (VEGF) is sequestered in platelets from tumor bearing mice but not in those from normal, heathy mouse platelets/plasma (labeled grey, control, top rows). In contrast, the right-hand panel indicates that nonspecific proteins such as albumin is not sequestered.

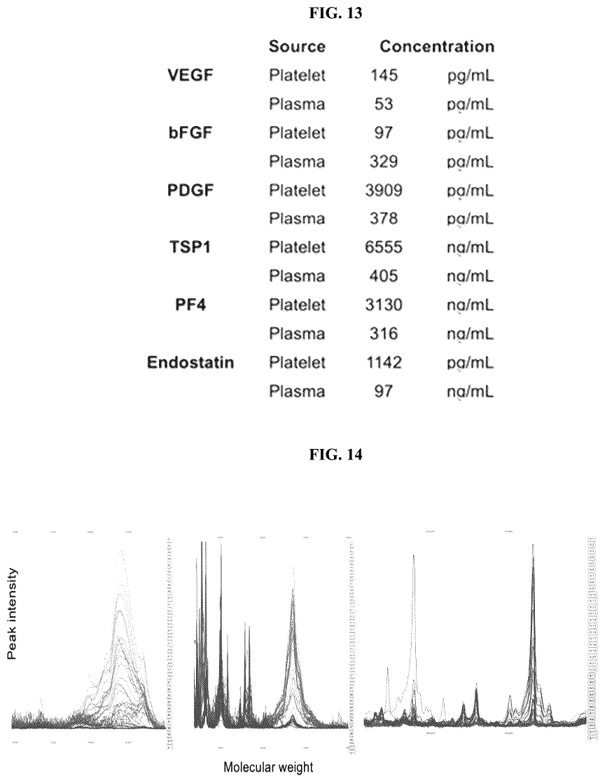

Next, platelets of healthy human individuals were found to contain predominantly inhibitors of angiogenesis. Platelets from 50 healthy human subjects (29 females and 21 males) ages (26 to 89 years, median 55±13years) were obtained and analyzed using commercial ELISA assays (R&D Systems, MN, USA) for specific proteins. A significant elevation of VEGF 215-Fold), PF-4 (516-fold), PDGF (914-fold), TSP-1 (813-fold), bFGF (17-fold) and endostatin (0.7-fold) in comparison to plasma, but more importantly, there was a balance of stimulators and inhibitors. (See, FIG. 13, which is a table showing that platelets contain both stimulators (VEGF, bFGF, PDGF) and inhibitors (PF4, endostatin) of angiogenesis). This balance was highly dynamic and changed early in cancer progression. For example, platelets from human subjects with colorectal carcinoma at the time of their primary resection (n=35) a multivariable, logistic regression modeling confirmed that PDGF (P=0.024), PF4 (P<0.0001), and VEGF (P=0.012) were independent predictors of CRC15. While platelets of normal human subjects show a predominance of angiogenesis inhibitors, whereas platelets of cancer patients contain a predominance of angiogenesis stimulators.

The balance of platelet sequestered stimulators and inhibitors was found to be sensitive to changes in the physiology of the human subjects. Platelet sequestration of cancer-related proteins can indicate lifestyle changes associated with better outcomes. Platelet-sequestered proteins were characterized in patients with localized prostate cancer at baseline (red group no lifestyle changes, blue with lifestyle changes) and following 6 months of intervention (lifestyle changes such as exercise, healthy food and regular sleep).

FIG. 14 shows SELDI-ToF analyses of platelets from subjects with localized prostate cancer undergoing positive lifestyle interventions and those undergoing watchful waiting without changes in lifestyle at 6 months post intervention. At baseline, there were no differences in the platelet protein profiles. Subjects who underwent lifestyle interventions (blue) showed a decline in cancer growth stimulators such as VEGF (peaks at 47 and 29 kD) and an increase in cancer growth inhibitors such as PF4 and CTAPIII (peaks at 7.4 and 9.3 kD). There was a clear upregulation of inhibitors and decrease of stimulators in the lifestyle interventions group. In contrast patients who made no changes to their lifestyle red showed the opposite trend at 6 months.

### Example 8: The Main Determinant of Whether a Protein is or is not Sequestered in Platelets is the Ability of a Protein to Bind to Glycosaminoglycans

In this Example, the ability of a protein to be sequestered in platelets was determined.

Under normal physiological conditions platelets express very high levels of enzymes that cleave glycosaminoglycans (GAGs). For example, an endo-glucuronidase preferentially cleaves heparan sulfate (HS) and heparin polysaccharides—heparanase—is expressed at very low levels in normal tissues but becomes over expressed in pathological conditions such as injury, cancer or inflammation. The best studied platelet protein—platelet factor 4 (PF4) is stored in platelet α-granules bound to the glycosaminogly can (GAG) chains of serglycin. Platelet serglycin is decorated with chondroitin/dermatan sulfate to which PF4 binds. More importantly, it has been shown that the affinity of PF4 for specific GAG subtypes modulates the local regulation of tumor associated angiogenesis. Because PF4 has higher affinity for endothelial cell-derived perlecan heparan sulfate chains than for the platelet-derived serglycin GAG chains it will bind to endothelia cell GAGS and prevent the binding of angiogenesis stimulators such as FGF2. Previously, it was shown that it is the growth factor affinity for GAGs that determines cell-cell interaction during wound healing or tumor growth.

FIG. 15A and FIG. 15B are graphs showing inhibition of the respective receptor does not inhibit platelet sequestration, but inhibition of heparin binding by surfen results in significant inhibition of protein sequestration by platelet a granules. FIG. 15A, illustrating FACS analysis for FGF, PF4, VEGF and TPO, shows identical platelet uptake of the growth factors in permeabilized platelets (black, left column of each pair) and in platelets exposed to the respective receptor inhibitor (grey, right column of each pair). In contrast, as shown in FIG. 15B, pretreatment of platelet rich plasma by surfen (blue, right column of each pair), a non-specific glycosaminoglycan inhibitor significantly inhibits growth factor uptake by platelets in all but thrombopoietin, which is the only growth factor tested that does not bind heparan sulfate. (Inh=inhibitor added, PF4 receptor, a splice variant of the chemokine receptor CXCR3, known as CXCR3B).

Apparently, the main determinant of whether a protein is or is not sequestered in platelets is the ability of a protein to bind to glycosaminoglycans such as heparan sulfate, chondroitin sulfate, serglycin or perlecan.

Because growth factors and angiogenesis regulatory proteins can be transported by platelet α-granules bound to GAGs without receptor activation or degradation, novel platelet anchoring ligand (PAL) were developed. Elsewhere in this disclosure a variety of PAL are disclosed. PAL1, for example, has the sequence of ERRIWFPYRRF (SEQ ID NO: 1); it has been shown to bind chondroitin sulfate (CS), the main GAG in platelet α-granule was developed.

### Example 9: Platelets have Distinct Types of α-Granules that Release Their Contents in a Temporally and Spatially Regulated Manner

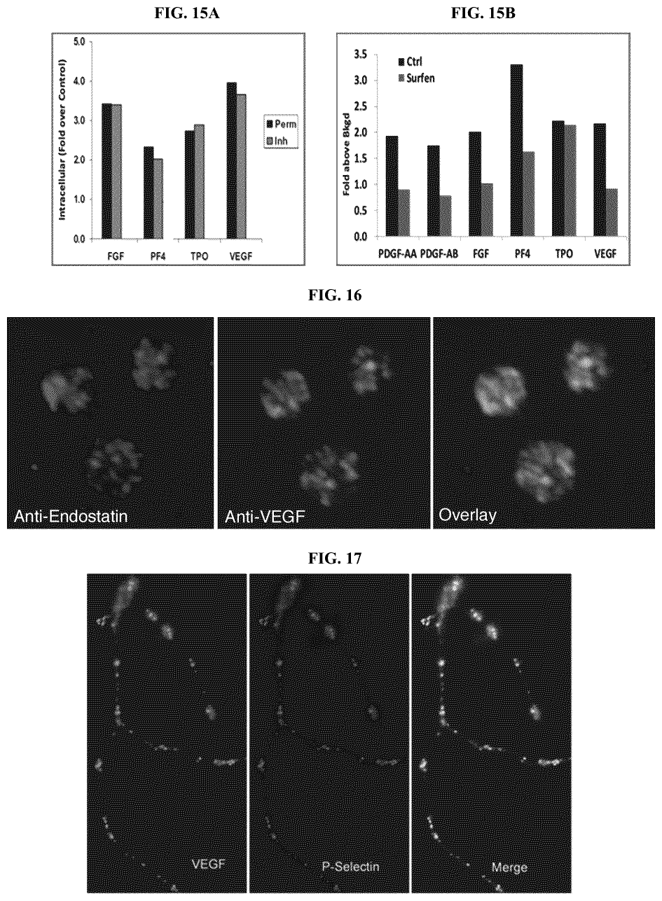

In this Example, characterization of distinct α-granule compartments is described. Platelets simultaneously loaded with VEGF (a stimulator of angiogenesis) and endostatin (an inhibitor of angiogenesis) will occupy separate α-granules. Using immunofluorescence microscopy, the localization of both angiogenesis inhibitors and stimulators in platelets and megakaryocytes were visualized. As shown in FIG. 16, double immunofluorescence microscopy revealed that endostatin (in red, left panel and overlay), an inhibitor of angiogenesis, and VEGF (in green, middle panel and overlay), an angiogenesis stimulator, are localized to separate α-granules (as shown in the overlay, right panel).

Further experiments identified these separate granules as either P-selectin associated (released early by the high-affinity thrombin receptor PAR1) or von Willebrand factor (VWF) associated (released by the low-affinity thrombin receptor PAR4). FIG. 17 are immunofluorescent images showing that a stimulator of angiogenesis localizes with P-selectin. After establishing that VEGF and endostatin were in separate organelles, the α-granules were subtyped. Antibodies that recognize specific platelet granules such anti-P selectin and anti-von Willebrand factor were used to label α-granules and anti-serotonin antibodies were used to label dense granules. Double immunofluorescence microscopy with antibodies against VEGF (in green, left panel) and antibody against the α-granule marker P-selectin (in red, middle panel) confirms that VEGF is localized to P-selectin α-granules (right panel, Merge).

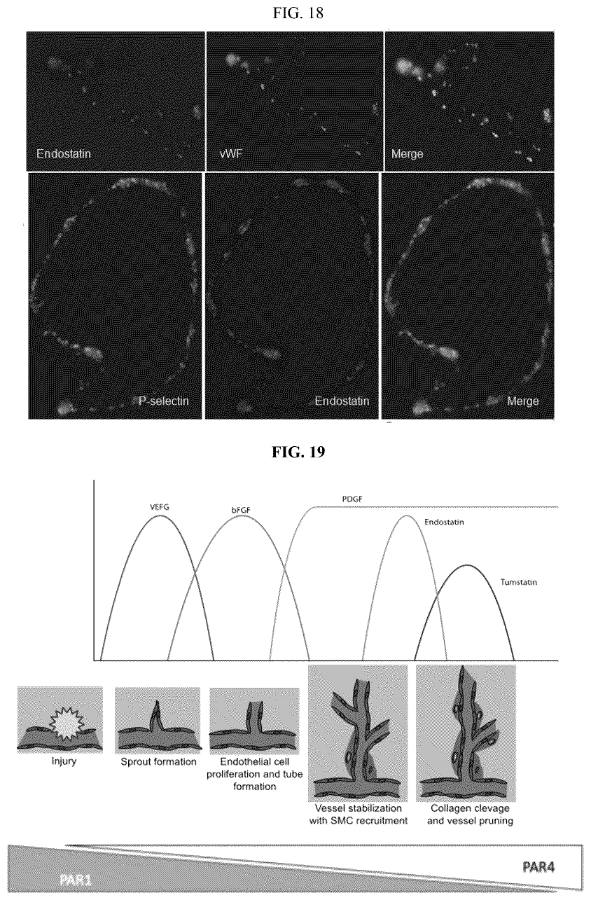

In contrast, endostatin did not colocalize to the P-selectin α-granules, but rather colocalized with the von Willebrand factor (vWF) α-granules. FIG. 18 are immunofluorescent images showing that endostatin is in a separate and distinct α-granule compartment and co-localizes with vWF (top row) rather than with P-selectin (bottom row). Double immunofluorescence staining with an antibody against von Willebrand Factor (vWF), an established protein in α-granules, demonstrates that endostatin is also contained in α-granules, but it does not co-localize with P-selectin confirming that it is in a distinctly different α-granule compartment.

The roles of two types of α-granules may be understood in the context of wound healing. In wound healing, immediately following injury pro-inflammatory cytokines and angiogenesis stimulating growth factors are needed. However, as the tissue heals more inhibitors of angiogenesis are released. FIG. 19 include schematics summarizing the sequential release of proteins in wounds healing and local concentration gradients of proteinase activated receptor 1 (PAR1) and PAR4. As shown, immediately following injury, an initial transient signals for vessel sprouting is induced by VEGF, followed by elongation and tube formation (due to bFGF), vessel stabilization through recruitment of pericytes (due to PDGF), and fmally vessel pruning (due to endostatin, tumstatin and other collagen and plasmin cleavage products). The process of normal wound healing takes approximately 7-10 days. Notably, this process reproduces the embryonal sequence of carefully orchestrated vessel formation through temporally and spatially controlled sequential protein release.

Without wishing to be bound by theory, a platelet's ability to release its content in a temporally and spatially controlled sequential, via distinct α-granule types, could be exploited in the field of therapeutic in which platelets are loaded with a first drug into a first α-granule type that has an early release profile and with a second drug into a second α-granule type that has a later release profile. For this, characteristics of the distinct α-granule types must be known and the means for selectively loading one α-granule type versus the other α-granule type must be established.

Apparently, platelets and endothelial cells are able to internalize growth factors (such as VEGF, bFGF and PDGF) due to the growth factor's ability to bind glycosaminoglycans (GAGs) such as Heparin Sulfate (HS) or Chondroitin Sulfate (CS).

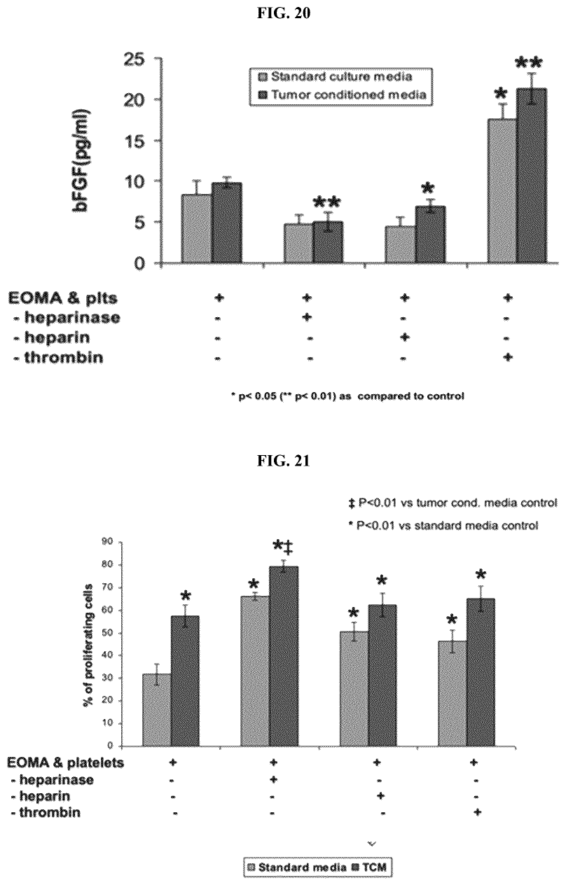

FIG. 20 is a graph showing sequestration of growth factors by glycosaminoglycans (GAGs) on the surface of murine hemangioendothelioma cells (EOMA). Here, the cells growing in monolayer tissue culture conditions either under standard media conditions or in surrogate tumor environment using tumor conditioned media, sequester bFGF onto the GAGS on platelets and endothelial cells, and thus remove it from the supernatant. Heparin and heparinase further enhance the phenomenon. However, in tumor microenvironments, simulated here by tumor conditioned media enriched with thrombin (right-most data columns), the bFGF and its derivatives are released from the GAGs into the supernatant and by extension into the tumor microenvironment. Values represent means and SE of 5 wells. These data confirm that the sequestration is heparin dependent and increases in the presence of thrombin.

Confirmation that growth factors are released from the provisional matrix formed by a platelet clot is supported by FIG. 21.

FIG. 21 is a graph showing proliferation of murine hemangioendothelioma cells (EOMA) in response to growth factors released from platelet formed provisional matrix. EOMA cells growing in monolayer tissue culture using standard media or tumor conditioned media sequester bFGF and other heparin sulfate (HS) binding growth factors, by anchoring them to GAGs on the membranes of EOMA cells and on the platelet provisional matrix. The liberation of the growth factors by heparinase increases the proliferative potential of endothelial cell, e.g., in tumor microenvironment. Values represent means and SE of 5 wells.

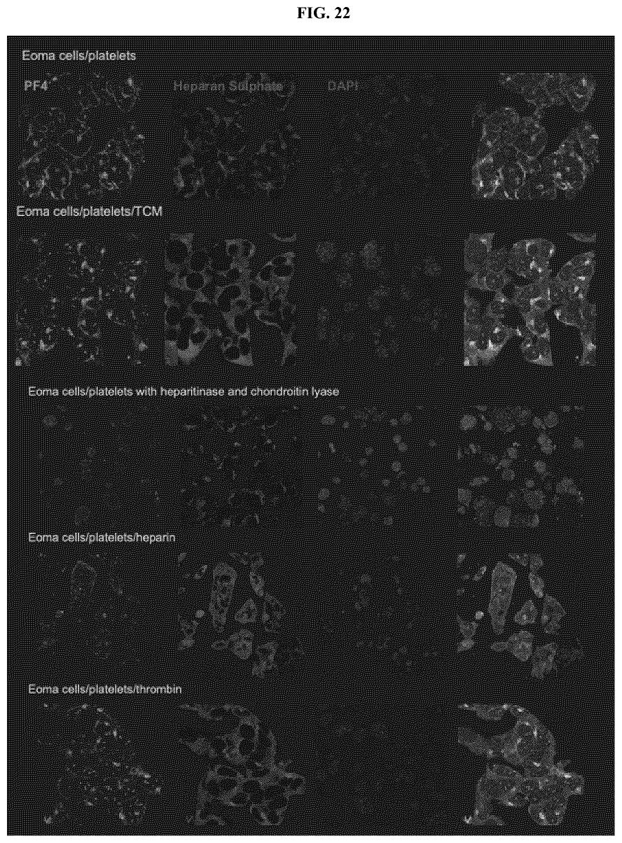

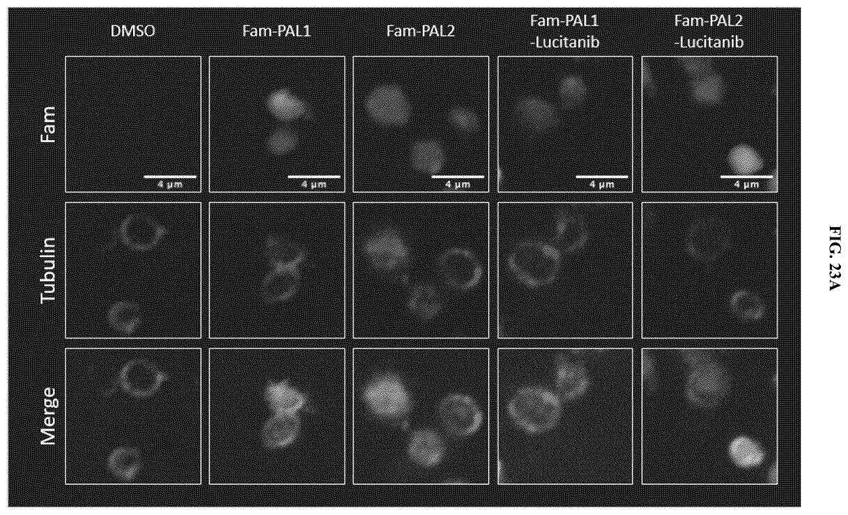

Platelet factor 4 (PF4) has one of the highest affinities for HS occurring in nature and can occupy GAG sites and displace other HS binding growth factors. As such, PF4 acts as an inhibitor of tumor growth. FIG. 22 are immunofluorescent images showing that platelets form a provisional matrix that can exchange proteins with endothelial cells upon tumor activation. Murine hemangioendothelioma cells (EOMA) cells were grown in standard media (DMEM+10% FBS), and normal platelets were added. Under normal conditions (top row), PF4 (the main content of platelets α-granules; green and left column) is seen at the periphery (i.e., along the membrane) of the EOMA cells as the platelets aggregate along the cell membrane. Heparan sulphate (red, second from left column) is distributed throughout the membranous surface of the endothelial cell, and DAPI (blue, second from right column) is the nuclear counterstain. In absence of tumor conditioned media, thrombin or other activators of endothelial cell, there is no clumping and aggregation of the platelets. Most of the PF4 remains in platelets and does not co-localize with endothelial cell GAGS (see right-most image of top row). However, upon incubation of EOMA cells and platelets in presence of tumor conditioned media (second row), the expression of HS is increased, and a thick platelet provisional matrix is formed as shown by the aggregation of PF4 on the HS rich surface EOMA cell; this platelet provisional matrix is used by EOMA cells for growth. This aggregation of platelet PF4 within the cells and the subsequent stimulation of endothelial cell growth is inhibited by chondroitinase and heparitinase (third row), by heparin (fourth row). On the other hand, the aggregation of platelet PF4 on the HS rich surface EOMA cells is reproduced by thrombin (bottom row).

The accumulation of α-granules within the provisional matrix can be exploited to load different drugs into the different compartments of α-granules; these are trapped in the platelet formed provisional matrix at the tumor site and locally released in a temporally and spatially controlled manner, e.g., by thrombin and its fragments, present at the tumor site.

### Example 10: PAL Conjugates Load into Platelets

In this Example, various conjugates (with a fluorescent marker alone or with a fluorescent marker and an illustrative active agent, here lucitanib) were loaded into platelets.

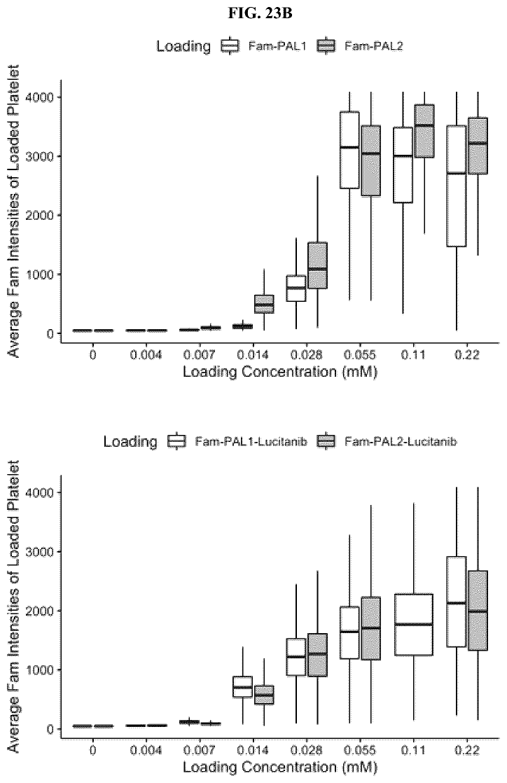

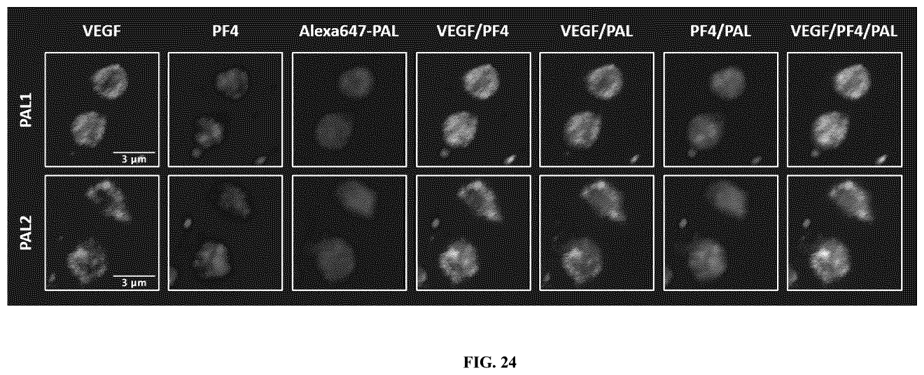

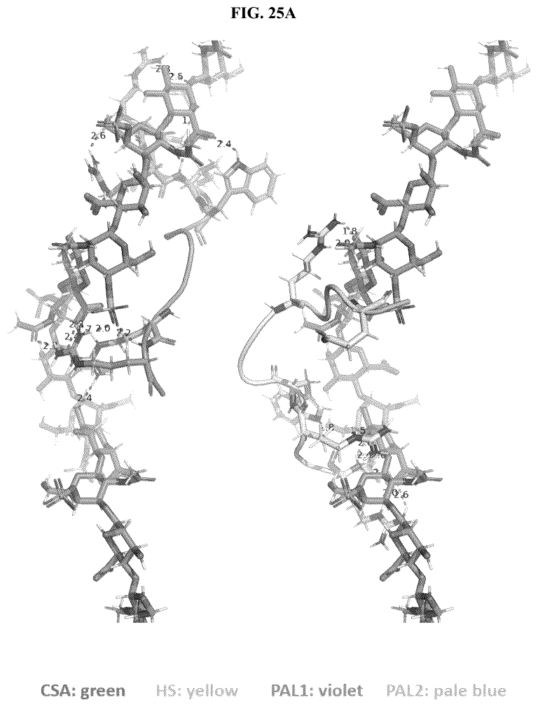

As shown in FIG. 23A, each of the Fam-PAL1, Fam-PAL2, Fam-PAL1-Lucitanib, and Fam-PAL2-Lucitanib load into platelets and without visibly harming the loaded platelets. When compared to the DMSO control, Fam-PAL1, Fam-PAL2, Fam-PAL1-Lucitanib, and Fam-PAL2-Lucitanib were internalized into platelets (green channel) while keeping platelet morphology intact and in a resting, fully functional state (purple) rather than becoming activated by the loading process which would make the platelets pro-coagulant.

FIG. 23B shows dose-responsive loading of Fam-PAL1 or Fam-PAL2 (top) and Fam-PAL1 -Lucitanib and Fam-PAL2-Lucitanib (bottom) into platelets. As shown, the doses of the conjugates ranged from 0.004 mM to 0.22 mM.

In this example, fresh platelet suspension in PBS were coincubated with different concentrations of the indicated compounds in 37° C. for 1 hour and were then separated by spinning at 800 g for 10 minutes. The loaded platelets were then fixed in 2% paraformaldehyde for 30 minutes at room temperature, permeabilized in 0.2% Triton-X 1% BSA in PBS for 30 minutes at room temperature, and blocked in 1% BSA in PBS for 30 minutes at room temperature, with three PBS washes between each step. Lastly, the platelets were seeded onto poly-lysine coated glass-bottom 384-well plates and immuno-stained with Rabbit-anti-human Tubulin and A647-donkey-anti-rabbit. The images were acquired using Nikon-A1 confocal microscope equipped with 60× oil-immersed objective, processed using ImageJ, and analyzed using CellProfiler. The graphs of FIG. 23B were plotted using R with >1,500 platelets analyzed for each group.

### Example 11: The Distinct Types of α-Granules can be Selectively Loaded

In this Example, differences and affinities of specific platelet anchoring sequences (PAL) for specifically loading different sub compartments of α-granules.

FIG. 24 are immunofluorescent images showing that PAL1 and PAL2 have different subcellular localizations, i.e., have a preference for distinct alpha-granules. Using VEGF as the marker of one alpha-granule set (green) and PF4 as the marker of the second alpha-granule set (red), the subcellular localization of Alexa647-labelled PAL1 and PAL2 (blue) were recorded on Nikon-Al confocal microscope. PAL1 and PAL2 were loaded to both the subsets of alpha-granules. However, PAL1 colocalized more to the VEGF subset indicated by the cyan color in VEGF/PAL merged column whereas PAL2 colocalized more to the PF4 channel shown by the purple color in PF4/PAL merged column. These data, together with the data shown in FIG. 4B (which shows that PAL1 binds CSA tighter than PAL2) and in FIG. 4C (which shows that PAL2 binds HS tighter than PAL1) suggests that the two types of α-granules are characterized by predominance of different GAG types.

Without wishing to be bound by theory, it appears that the PAL-conjugates first internalize within a platelet and then localize into a preferred granule type, based on the specific PAL sequence.

The images FIG. 24 were acquired using Nikon-A1 confocal microscope equipped with a 60× oil immersed objective. PAL1 and PAL2 partially colocalize with both subsets of the alpha-granules as indicated by the cyan and purple pixels in the merged images. To quantify the difference between PAL1 and PAL2, the fractionation of loaded platelets were performed

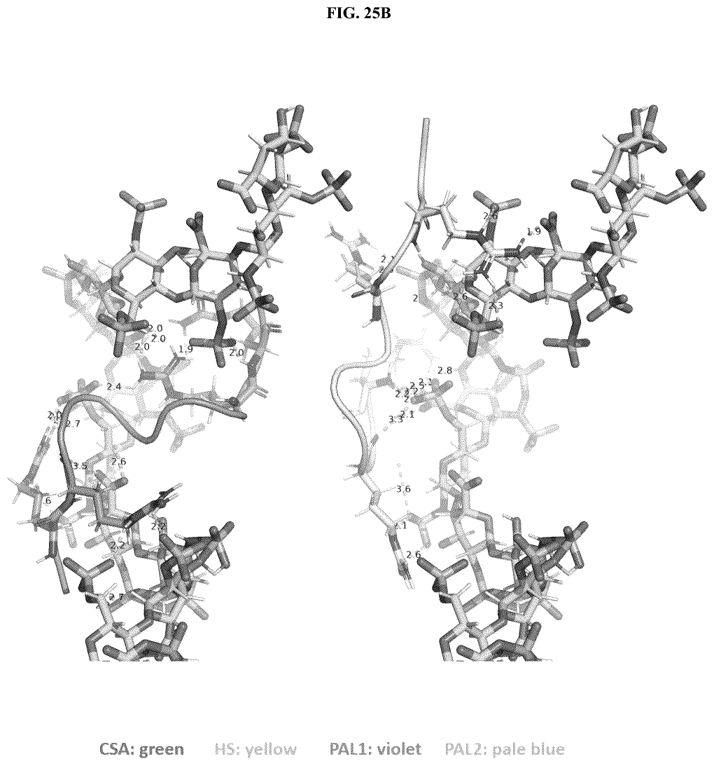

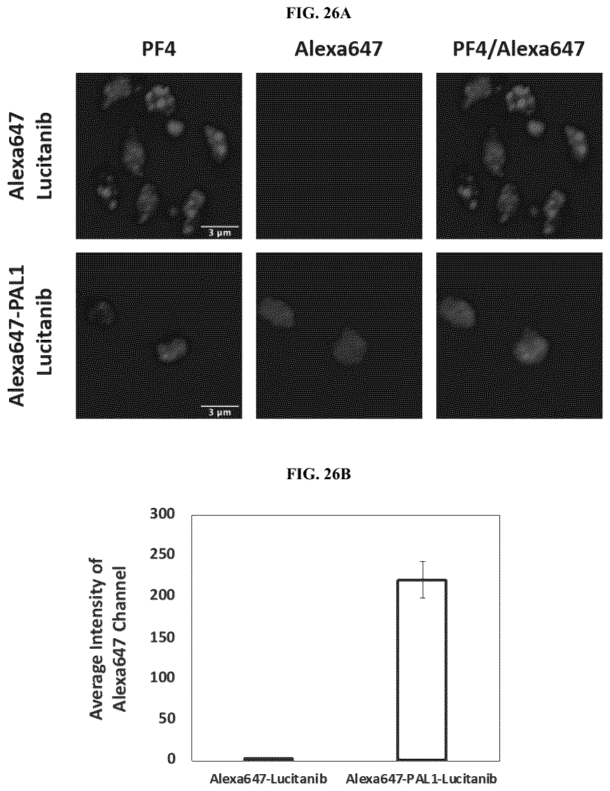

To further understand the mechanisms that leads to different subcellular localizations between Fam-PAL1 (violet in FIG. 25A and FIG. 25B) and Fam-PAL2 (pale blue in FIG. 25A and FIG. 25B), these two peptides were docked onto CSA (green in FIG. 25A) or HS (yellow in FIG. 25B) structures separately. The dotted lines indicate the intermolecular contacts that are key for the interactions. Overall both PAL1 and PAL2 use their Arginines to make contacts with GAGs. When binding to CSA, both PAL1 and PAL2 stay bound on one side of the molecule and make salt bridges and hydrogen bonds with it, which is consistent with their comparable binding affinities to CSA. As shown in FIG. 25B, PAL2 lays to the side of HS whereas PAL1 follows HS's groove; these distinct associations may lead to the two PAL's different affinities for HS. These structure models are consistent with observations made from bench-top experiments including Isothermal calorimetry, FPLC, and microscopy. These models, in view of the experimental data also shed light ability to exploit the different features of PAL1 and PAL2 to load multiple therapeutic reagents into platelets.

The knowledge that the α-granules types are characterized by predominance of different GAG types can be used to selectively load drugs into a specific type of α-granule.

FIG. 26A and FIG. 26B show that when PAL1 (SEQ ID NO: 1) is conjugated with a small molecule, the PAL1 can guide the respective molecule into a platelet α-granule. Platelets were co-incubated with Alexa647-labled Lucitanib or with Alexa647-PAL1 conjugated Lucitanib for one hour at 37° C. The platelets were then spun for 10-minutes at 800 g, fixed in 2% paraformaldehyde, and settled onto glass coverslips. After permeabilization, immunofluorescence staining was performed against PF4 in platelets and further stained with Alexa568-secondary antibody. The images were collected through a Nikon-Al laser-scanning microscope equipped with a 60× oil objective. FIG. 26A includes representative images in which immunofluorescence for PF4 was shown in red and Alexa647 was shown in blue. The merged PF4/Alexa647 channel displayed the colocalization of PF4 and Alexa647-labelled PAL1 conjugated-Lucitanib. FIG. 26B is a graph showing the averaged intensities of Alexa647 channel for >800 ROIs in each group were analyzed using ImageJ and the error bar is the standard deviation of the mean.

These data show that a PAL1-drug product can be loaded into α-granules and, particularly, into the CS, P-Selectin type granules.

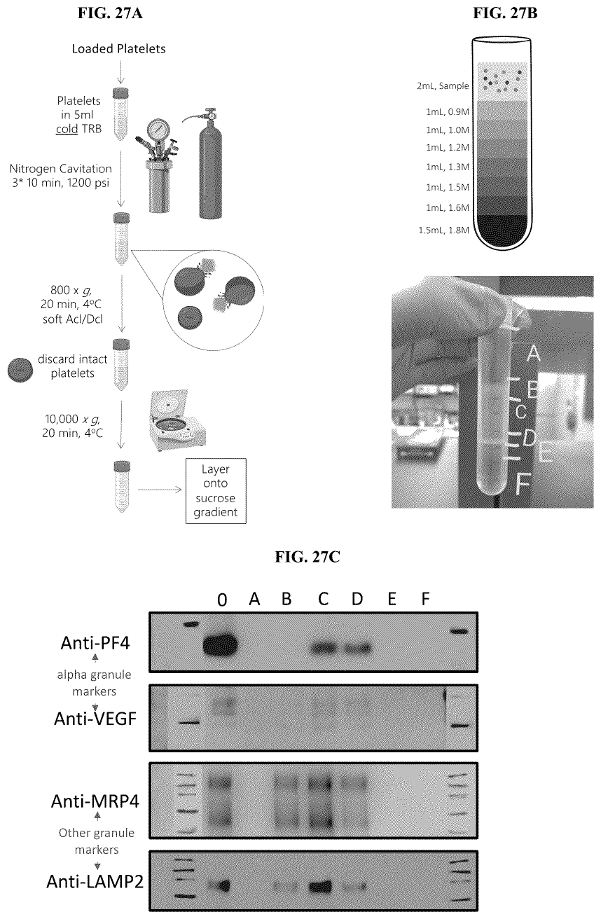

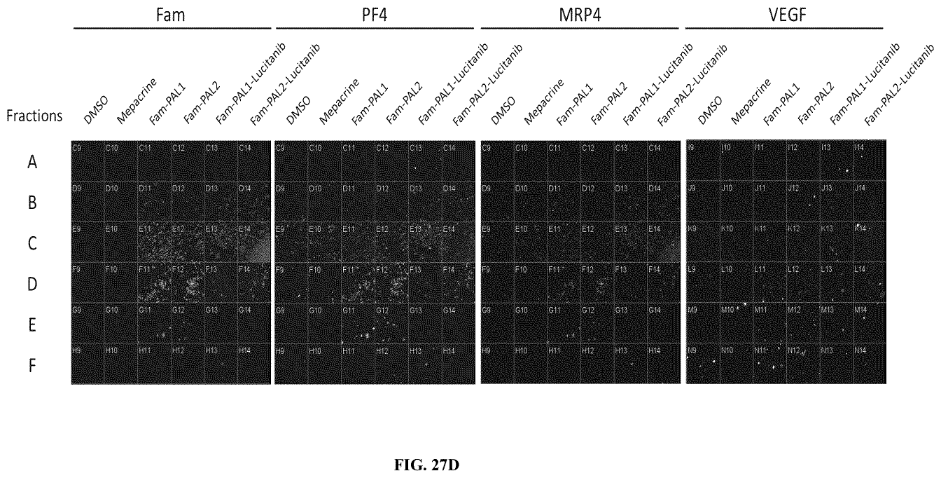

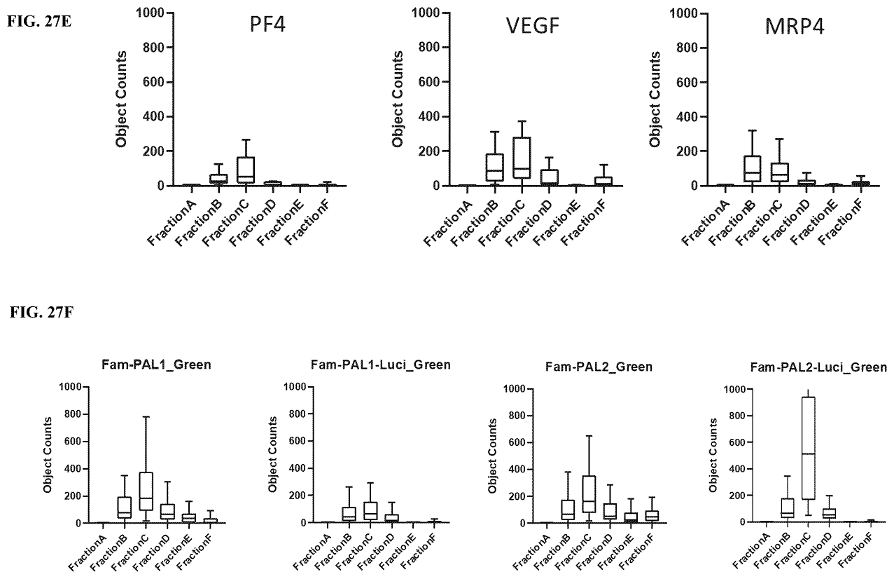

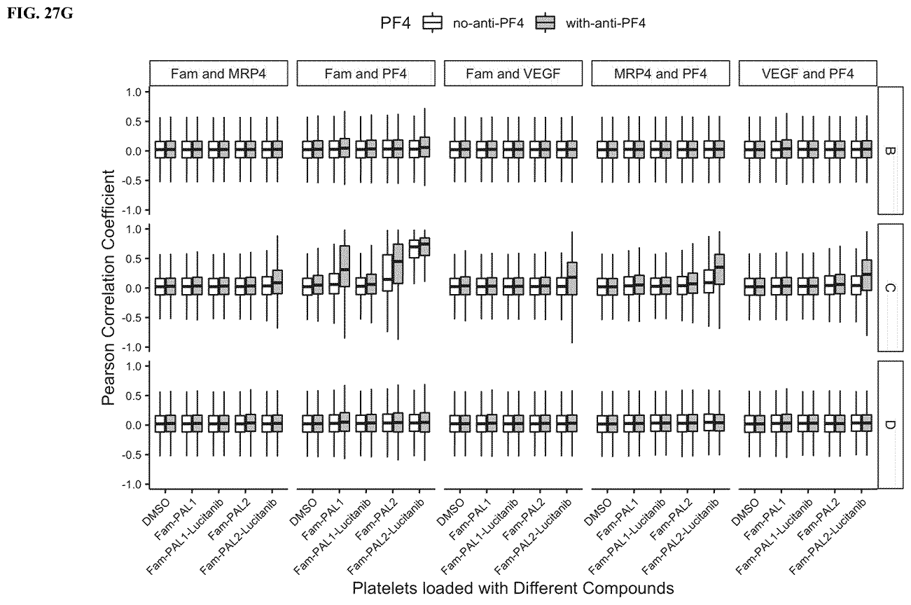

FIG. 27A to FIG. 27C demonstrate methods for fractionating platelet granules and show that different granules can be distinguished by protein markers. FIG. 27A is a flow chart illustrating steps in fractionating platelet granules. The fmal step is a layered sucrose gradient with separated platelet granules, shown in FIG. 27B with top image a cartoon showing the quantified gradient and the bottom image showing the different gradation that are loaded separately onto the gel that is represented in FIG. 27C. FIG. 27C are western blots of granule fractions from platelets that were loaded using DMSO as control. PF4 and VEGF are markers for alpha-granules and MRP4 and LAMP2 are markers for other storage granules including lysosomes and dense granule. The results show that most of the granules are enriched in fractions B, C and D.

To further investigate the subcellular localization of PAL1 and PAL2 and their conjugates, granule fractions from platelets loaded with different compounds were fixed and seeded in poly-lysine coated glass-bottom 384-well plates. As shown in FIG. 27D, immunostaining was carried out in two sets: one is with PF4 (yellow) and MRP4 (red) and the other is with PF4 (yellow) and VEGF (red). The images were acquired using high-throughput confocal microscope Phenix equipped with a 60× water-immersed objective. This meta-graph shows representative images from different wells and different channels as indicated. As shown in FIG. 27E, particle analysis for the images acquired on all the fractions collected from DMSO-treated platelets (shown in FIG. 27D) confirmed the observation of western blots that the markers PF4, VEGF, and MRP4 are enriched in fractions B, C, and D. Also, as shown in FIG. 27F, particle analysis for the green channel of the images (which demonstrates localization the Fam-PAL1 and the Fam-PAL1-Lucitanib conjugate or the Fam-PAL2 Fam-PAL2-Lucitanib conjugate acquired on all the fractions collected from platelets that were loaded with different compounds (as indicated) demonstrated that Fam-PAL1 and Fam-PAL2 and their conjugates were also enriched in fractions B, C and D. Finally, in order to quantify the subcellular localization of Fam-PAL1 or Fam-PLA2 and their conjugates, Pearson Correlation Analysis (PCA) were performed on all the images from fractions B, C and D using CellProfiler. See, FIG. 27G. In brief, for each comparing pair, the images from the two channels were merged and individual granules were segmented. PCA analysis was then carried out on the selected regions of interest (ROIs). The boxplot was graphed using R and each group contains >5,000 ROIs. As previously observed, fraction C gives the most localization information. The facet boxplot suggests that Fam-PAL1 and Fam-PAL2 and their conjugates primarily target the PF4-enriched subset of alpha granule. Fam-PAL2 and its conjugates also target on other granules that are enriched with VEGF or MRP4. Of note, Fam-PAL2 may have shifted Fam's excitation and emission; thus, to avoid the potential bleeding through from the green channel to red channel, no-anti-PF4 condition (white boxes) was employed as negative control.

These results indicate the potential to load multiple reagents into platelets using PAL1 and PAL2 simultaneously.

### Example 12: Illustrative Methods for Manufacturing an Isolated Platelet Loaded with Two Compounds, with Each Compound Being Loaded into a Distinct α-Granule Type

In this example, an isolated platelet is loaded with two compounds of the present disclosure, with each compound being loaded into a distinct α-granule type.

An isolated platelet is obtained. The platelet may be a synthetic platelet, an allogeneic platelet, an autologous platelet, or a modified heterologous platelet. In embodiments, the platelet is obtained from platelet rich plasma.

The platelet is contacted in vitro or ex vivo with a first compound of the present disclosure. The first compound comprises a first agent and a first polypeptide. The first polypeptide comprises a PAL1 (SEQ ID NO: 1) glycosaminoglycan (GAG)-binding peptide which can bind a GAG in an α-granule of a platelet. The PAL1 preferentially binds, at least, to chondroitin sulfate (CS) on a first type of α-granule, e.g., a P-Selectin type of α-granule.

The platelet is also contacted in vitro or ex vivo with a second compound of the present disclosure. The second compound comprises a second agent and a second polypeptide. The second polypeptide comprises a PAL2 (SEQ ID NO: 2) glycosaminoglycan (GAG)-binding peptide which can bind a GAG in an α-granule of a platelet. The PAL2 preferentially binds, at least, to heparin sulfate (HS) on a second type of α-granule, e.g., a von Willebrand factor (VWF) type of α-granule.

In embodiments, the first compound and the second compound are loaded sequentially. In alternate embodiments, the compound and the second compound are loaded simultaneously.

Contact continues at a suitable temperature, media composition (including salt concentration, pH, nutrients), and length of time until the compounds are internalized by the respective α-granule types of the platelet. As such, a loaded platelet is obtained. Often the temperature is the body temperature from which a platelet is obtained or to be administered, e.g., 37° C. Similarly, the pH of the composition is near the pH of blood/plasma from which a platelet is obtained or to be administered, e.g., a pH of about 7.4.

Any agent listed in the present disclosure or known in the art may be used in this example. The agent may be an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

In some embodiments, the first agent and the second agent may, independently, be one of an EGFR inhibitor (e.g., Cetuximab), a VEGF inhibitor (e.g., Bevacizumab), a PDL1 inhibitor (e.g., Pembrolizumab), an FN1 inhibitor (e.g., Ocriplasmin), a multikinase inhibitor (e.g., regorafenib), a FGFR2 antagonist (e.g., thalidomide), thrombin and its analogues, CSF3R agonist (e.g., Filgrastim), PSMB5 inhibitor (e.g., Bortezomib), fumagillin, or an ALK/ROS1/NTRK inhibitor (e.g., crizotinib).

The first agent and the second agent may be the same or may be different.

As examples, the first and second agents may be: a VEGF inhibitor (e.g., Bevacizumab) and a PDL1 inhibitor (e.g., Pembrolizumab); or an EGFR inhibitor (e.g., Cetuximab) and a multikinase inhibitor (e.g., regorafenib); or fumagillin and a multikinase inhibitor (e.g., regorafenib).

Preferably, an isolated platelet comprises 1 to 1000 copies of the first compound and comprises 1 to 1000 copies of the second compound.

The loaded platelets thus manufactured may be combined with one or more pharmaceutically acceptable excipients to produce a pharmaceutical composition.

In some embodiment, a third compound comprising a third polypeptide and a third agent, e.g., an EGFR inhibitor (e.g., Cetuximab) and a multikinase inhibitor (e.g., regorafenib), and an ALK/ROS1/NTRK inhibitor (e.g., crizotinib) may be combined.

Additionally, a pharmaceutical composition may be produced by combining a plurality of platelets each comprising different first and second compounds along with one or more pharmaceutically acceptable excipients. Any first and/or second agents mentioned above and any combinations thereof may be used.

### Example 13: Illustrative Methods for Treating a Disease or Disorder by Administering to a Subject Isolated Platelets Loaded with Two or More Compounds, with Each Compound Being Loaded into a Distinct α-Granule Type

In this example, isolated platelets loaded with two or more compounds of the present disclosure, with each compound being loaded into a distinct α-granule type, are administered to a subject in need, e.g., who has a disease or a disorder.

Here, a subject in need is administered (e.g., by infusion or injection) a therapeutically effective amount of one or more pharmaceutical compositions, each comprising platelets loaded with two or more compounds, with each compound being loaded into a distinct α-granule type, of the present disclosure.

A platelet in the composition comprises, at least, a first compound and a second compound of the present disclosure. The first compound comprises a first agent and a first polypeptide. The first polypeptide comprises a PAL1 (SEQ ID NO: 1) glycosaminoglycan (GAG)-binding peptide which can bind a GAG in an α-granule of a platelet. The PAL1 preferentially binds, at least, to chondroitin sulfate (CS) on a first type of α-granule, e.g., a P-Selectin type of α-granule. The second compound comprises a second agent and a second polypeptide. The second polypeptide comprises a PAL2 (SEQ ID NO: 2) glycosaminoglycan (GAG)-binding peptide which can bind a GAG in an α-granule of a platelet. The PAL2 preferentially binds, at least, to heparin sulfate (HS) on a second type of α-granule, e.g., a von Willebrand factor (VWF) type of α-granule.

In some embodiments, a platelet in the composition comprises a third compound. The third compound comprises a third agent and a third polypeptide, with the third polypeptide comprising a third glycosaminoglycan (GAG)-binding peptide which is capable of binding a GAG in a third alpha granule type of a platelet. The third GAG-binding peptide preferentially binds serglycin, perlecan, dermatan sulfate, keratan sulfate, and/or GPIIb/IIIa.

Any agent listed in the present disclosure or known in the art may be used in this example The first, second, or third agent may, independently, be an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

In some embodiments, the two compounds may, independently, comprise an agent selected from EGFR inhibitor (e.g., Cetuximab), a VEGF inhibitor (e.g., Bevacizumab), a PDL1 inhibitor (e.g., Pembrolizumab), an FN1 inhibitor (e.g., Ocriplasmin), a multikinase inhibitor (e.g., regorafenib), a FGFR2 antagonist (e.g., thalidomide), thrombin and its analogues, CSF3R agonist (e.g., Filgrastim), PSMB5 inhibitor (e.g., Bortezomib), fumagillin, and an ALK/ROS1/NTRK inhibitor (e.g., crizotinib). In embodiments, with a third compound, the third agent may again be selected from this list.

In embodiments, when at least three compounds are used, a first, second, and third agent may be an EGFR inhibitor (e.g., Cetuximab) and a multikinase inhibitor (e.g., regorafenib), and an ALK/ROS1/NTRK inhibitor (e.g., crizotinib); this may be used for treating non-small cell lung cancer.

The platelets may be loaded with a combination compounds of the present disclosure. As examples, a first and second agent may be a VEGF inhibitor (e.g., Bevacizumab) and a PDL1 inhibitor (e.g., Pembrolizumab); this may be used for treating pancreatic cancer. Also, a first and second agent may be an EGFR inhibitor (e.g., Cetuximab) and a multikinase inhibitor (e.g., regorafenib); this may be used for treating lung cancer. A first and second agent may be a multikinase inhibitor (e.g., regorafenib) and fumagillin; this may be used for treating pancreatic cancer, lung cancer, or colon Cancer.

The subject may further be administered a second pharmaceutical composition comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, and/or a metalloproteinase, a peroxidase, and/or a phosphohydrolase. The second pharmaceutical composition promotes release of the compound from a platelet. The second pharmaceutical composition may be administered after the pharmaceutical composition is administered, e.g., at least twice before the second pharmaceutical composition is administered.

Alternately, the subject may be administered a second pharmaceutical composition and/or a third composition each comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, and/or a metalloproteinase, a peroxidase, and/or a phosphohydrolase. The second pharmaceutical composition promotes release of the first compound from a first type of α-granule and the third pharmaceutical composition promotes release of the second compound from a second type of α-granule. The second and third pharmaceutical compositions may be administered after the pharmaceutical composition is administered, e.g., at least twice before the second pharmaceutical composition is administered. The second composition may be administered after the third pharmaceutical composition is administered, or vice versa.

A subject may be administered additional therapeutic agents in conjunction with the pharmaceutical compositions comprising loaded platelets. As an example, a subject may be administered platelets loaded with a VEGF inhibitor (e.g., Bevacizumab) and also administered Remdesivir; this may be used to treat acute respiratory distress syndrome (ARDS), perhaps associated with COVID. A subject may be administered platelets loaded with one or both of a multikinase inhibitor (e.g., regorafenib) and fumagillin, and also administered a low-dose chemotherapy; this may be used for treating pancreatic cancer, lung cancer, or colon cancer. A subject may be administered platelets loaded with one or both of an EGFR inhibitor (e.g., Cetuximab) and a multikinase inhibitor (e.g., regorafenib) and also administered a low-dose chemotherapy; this may be used for treating lung cancer. A subject may be administered platelets loaded with one or both or all three of an EGFR inhibitor (e.g., Cetuximab), a multikinase inhibitor (e.g., regorafenib), and an ALK/ROS1/NTRK inhibitor (e.g., crizotinib) and also administered a low-dose chemotherapy; this may be used for treating non-small cell lung cancer.

The subject in need may have a disease or disorder selected from a cancer or an injury Inflammation may be a symptom of the disease or disorder. The disease or disorder may be a side effect of an implant, graft, stent, or prosthesis. The disease or disorder may be caused by a defective gene.

### Example 14: Illustrative Methods for Treating a Disease or Disorder by Administering to a Subject Two or More Compounds of the Present Disclosure

In this example, two or more compounds of the present disclosure are administered to a subject in need, e.g., who has a disease or a disorder.

Here, a subject in need is administered (e.g., by infusion or injection) therapeutically effective amount of a pharmaceutical composition comprising two or more compounds of the present disclosure. In this method, the two or more compounds are loaded into a platelet in vivo.

The first compound comprises a first agent and a first polypeptide. The first polypeptide comprises a PAL1 (SEQ ID NO: 1) glycosaminoglycan (GAG)-binding peptide which can bind a GAG in an α-granule of a platelet. The PAL1 preferentially binds, at least, to chondroitin sulfate (CS) on a first type of α-granule, e.g., a P-Selectin type of α-granule. The second compound comprises a second agent and a second polypeptide. The second polypeptide comprises a PAL2 (SEQ ID NO: 2) glycosaminoglycan (GAG)-binding peptide which can bind a GAG in an α-granule of a platelet. The PAL2 preferentially binds, at least, to heparin sulfate (HS) on a second type of α-granule, e.g., a von Willebrand factor (VWF) type of α-granule.

In some embodiments a third compound is loaded into the platelet. The third compound comprises a third agent and a third polypeptide, with the third polypeptide comprising a third glycosaminoglycan (GAG)-binding peptide which is capable of binding a GAG in a third alpha granule type of a platelet. The third GAG-binding peptide preferentially binds serglycin, perlecan, dermatan sulfate, keratan sulfate, and/or GPIIb/IIIa.

Any agent listed in the present disclosure or known in the art may be used in this example The first, second, or third agent may, independently, be an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

In some embodiments, the two compounds may, independently, comprise an agent selected from an EGFR inhibitor (e.g., Cetuximab), a VEGF inhibitor (e.g., Bevacizumab), a PDL1 inhibitor (e.g., Pembrolizumab), an FN1 inhibitor (e.g., Ocriplasmin), a multikinase inhibitor (e.g., regorafenib), a FGFR2 antagonist (e.g., thalidomide), thrombin and its analogues, CSF3R agonist (e.g., Filgrastim), PSMB5 inhibitor (e.g., Bortezomib), fumagillin, or an ALK/ROS1/NTRK inhibitor (e.g., crizotinib).

The subject may be administered more than two compounds; the additional compounds may have an agent selected from the immediately above list or from any agent known in the art, e.g., an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

In embodiments, when at least three compounds are used, a first, second, and third agent may be an EGFR inhibitor (e.g., Cetuximab) and a multikinase inhibitor (e.g., regorafenib), and an ALK/ROS1/NTRK inhibitor (e.g., crizotinib); this may be used for treating non-small cell lung cancer.

The subject may further be administered a second pharmaceutical composition comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, and/or a metalloproteinase, a peroxidase, and/or a phosphohydrolase. The second pharmaceutical composition promotes release of the compounds from a platelet. The second pharmaceutical composition may be administered after the pharmaceutical composition is administered, e.g., at least twice before the second pharmaceutical composition is administered.

Alternately, the subject may be administered a second pharmaceutical composition and/or a third composition each comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, and/or a metalloproteinase, a peroxidase, and/or a phosphohydrolase. The second pharmaceutical composition promotes release of the first compound from a first type of α-granule and the third pharmaceutical composition promotes release of the second compound from a second type of α-granule. The second and third pharmaceutical compositions may be administered after the pharmaceutical composition is administered, e.g., at least twice before the second pharmaceutical composition is administered. The second composition may be administered after the third pharmaceutical composition is administered, or vice versa.

A subject may be administered additional therapeutic agents in conjunction with the pharmaceutical compositions comprising a compound of the present disclosure. Additional therapeutic agents may be Remdesivir and/or a low-dose chemotherapy.

The subject in need may have a disease or disorder selected from a cancer or an injury Inflammation may be a symptom of the disease or disorder. The disease or disorder may be a side effect of an implant, graft, stent, or prosthesis. The disease or disorder may be caused by a defective gene.

What is claimed is:

1. A composition comprising:
a first compound comprising a first agent and a first polypeptide, wherein the first polypeptide comprises a first glycosaminoglycan (GAG)-binding peptide which is capable of binding a GAG in a first alpha granule type of a platelet; and
a second compound comprising a second agent and a second polypeptide, wherein the second polypeptide comprises a second glycosaminoglycan (GAG)-binding peptide which is capable of binding a GAG in a second alpha granule type of the platelet.

2. The composition of claim 1, wherein the first GAG-binding peptide preferentially binds to chondroitin sulfate (CS) and the second GAG-binding peptide preferentially binds to heparan sulfate (HS).

3. The composition of claim 2, wherein the first GAG-binding peptide preferentially binds to chondroitin sulfate A (CSA) and does not preferably bind to heparan sulfate (HS).

4. The composition of any one of claims 1 to 3, wherein the first alpha granule type is a P-selectin associated granule and the second alpha granule type von Willebrand factor associated granule.

5. The composition of any one of claims 1 to 4, wherein contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin derivative such as tissue plasminogen activator (tPA).

6. The composition of any one of claims 1 to 5, wherein the contents of the first alpha granule type are released at a lower concentration of thrombin than the concentration of thrombin needed to provide release of the contents of the second alpha granule type.

7. The composition of any one of claims 1 to 6, wherein the contents first alpha granule type is released before the contents of the second alpha granule type are released.

8. The composition of any one of claims 1 to 7, wherein the first and the second GAG-binding peptides are each between about 8 amino acids and about 14 amino acids in length.

9. The composition of any one of claims 1 to claim 8, wherein one or both of the first and the second GAG-binding peptides comprises at least one charged amino acid.

10. The composition of any one of claims 1 to claim 9, wherein both of the first and the second GAG-binding peptides comprise at least one charged amino acid.

11. The composition of any one of claims 1 to 10, wherein one or both of the first and the second GAG-binding peptides comprises at least one proline, arginine, and/or isoleucine.

12. The composition of claim 11, wherein both of the first and the second GAG-binding peptides comprise at least at least one proline, arginine, and/or isoleucine.

13. The composition of any one of claims 1 to 12, wherein the first and the second GAG-binding peptides independently comprise an amino acid sequence that is at least about 70% identical to one of SEQ ID NO: 1 to SEQ ID NO: 13.

14. The composition of any one of claims 1 to 13, wherein the first and the second GAG-binding peptides independently comprise an amino acid sequence that is at least about 80% identical to one of SEQ ID NO: 1 to SEQ ID NO: 13.

15. The composition of any one of claims 1 to 14, wherein the first and the second GAG-binding peptides independently comprise an amino acid sequence that is at least about 90% identical to one of SEQ ID NO: 1 to SEQ ID NO: 13.

16. The composition of any one of claims 1 to 15, wherein the first and the second GAG-binding peptides independently comprise a charged amino acid at position 1, position 4, position 7, or position 9 with respect to any one of SEQ ID NO: 1 to SEQ ID NO: 13.

17. The composition of any one of claims 1 to 16, wherein the first and the second GAG-binding peptides independently comprise a proline, arginine, and/or isoleucine at position 1, position 4, position 7, and/or position 9 with respect to any one of SEQ ID NO: 1 to SEQ ID NO: 13.

18. The composition of any one of claims 1 to 17, wherein the first and the second GAG-binding peptides independently comprise at least 10 amino acids.

19. The composition of any one of claims 1 to 18, wherein the first and/or the second GAG-binding peptides independently comprise 11 amino acids.

20. The composition of any one of claims 1 to 19, wherein the first and the second GAG-binding peptides independently consist of 11 amino acids.

21. The composition of any one of claims 1 to 20, wherein the first and the second GAG-binding peptides independently comprise the amino acid sequence of one of SEQ ID NO: 1 to SEQ ID NO: 13.

22. The composition of any one of claims 1 to 20, wherein the first GAG-binding peptide comprises an amino acid sequence that is at least 90% identical to SEQ ID NO: 1 and the second GAG-binding peptide comprises an amino acid sequence that is at least 90% identical to SEQ ID NO: 2.

23. The composition of claim 22, wherein the first GAG-binding peptide comprises the amino acid sequence of SEQ ID NO: 1 and the second GAG-binding peptide comprises the amino acid sequence of SEQ ID NO: 2.

24. The composition of claim 22 or claim 23, wherein the first GAG-binding peptide consists of the amino acid sequence of SEQ ID NO: 1 and the second GAG-binding peptide consists of the amino acid sequence of SEQ ID NO: 2.

25. The composition of any one of claims 1 to 24, wherein the first polypeptide consists of the first GAG-binding peptide and the second polypeptide consists of the second GAG-binding peptide.

26. The composition of any one of claims 1 to 25, wherein the N-terminal of the first polypeptide is directly or indirectly linked to the first agent and/or the N-terminal of the second polypeptide is directly or indirectly linked to second first agent.

27. The composition of any one of claims 1 to 26, wherein the C-terminal of the first polypeptide is directly or indirectly linked to the first agent and/or the C-terminal of the second polypeptide is directly or indirectly linked to second first agent.

28. The composition of any one of claims 1 to 27, wherein the first agent is indirectly linked to the first polypeptide via a first linker and/or the second agent is indirectly linked to the second polypeptide via a second linker.

29. The composition of claim 28, wherein the first linker and/or the second each comprise one or more atoms.

30. The composition of claim 28 or 29, wherein the first linker and/or the second each comprise a polymer of repeating units.

31. The composition of any one of claims 28 to 30, wherein the first linker and/or the second linker each comprise a chain of amino acids.

32. The composition of any one of claims 1 to 31, wherein the first agent is directly linked to the first polypeptide and/or the second agent is directly linked to the second polypeptide.

33. The composition of any one of claims 1 to 31, wherein the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

34. The composition of any one of claims 1 to 33, wherein the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

35. The composition of claim 34, wherein the first agent and/or the second agent comprises an antibody or a fluorescent moiety.

36. The composition of any one of claims 1 to 35, wherein the first agent and/or the second agent is harmful to mammalian cells and/or is toxic to a subject and/or the first agent and/or the second agent is susceptible to degradation when administered directly into the bloodstream of a subject.

37. The composition of any one of claims 1 to 36, wherein the first compound and/or the second compound further comprises a fluorescent moiety.

38. The composition of any one of claims 1 to 37, wherein the first GAG-binding peptide and/or the second GAG-binding peptide also preferentially binds serglycin, perlecan, dermatan sulfate, keratan sulfate, and/or GPIIb/IIIa.

39. The composition of any one of claims 1 to 38, further comprising a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

40. An isolated platelet comprising:
at least one copy of a first compound comprising a first agent and a first polypeptide, wherein the first polypeptide comprises a first glycosaminoglycan (GAG)-binding peptide which is capable of binding a GAG in a first alpha granule type of a platelet; and
at least one copy of a second compound comprising a second agent and a second polypeptide, wherein the second polypeptide comprises a second glycosaminoglycan (GAG)-binding peptide which is capable of binding a GAG in a second alpha granule type of the platelet.

41. The isolated platelet of claim 40, wherein the platelet is a synthetic, an allogeneic, an autologous, or a modified heterologous platelet.

42. The isolated platelet of claim 41, wherein the platelet is an autologous platelet.

43. The isolated platelet of claim 41, wherein the platelet is an allogeneic platelet.

44. The isolated platelet of claim 42 or claim 43, wherein the platelet is obtained from platelet rich plasma.

45. The isolated platelet of any one of claims 40 to 44, wherein the platelet comprises 1 to 1000 copies of the first compound and 1 to 1000 copies of the second compound.

46. The isolated platelet of claim 45, wherein the 1 to 1000 copies of the first compound are loaded into a first alpha granule type of a platelet and the 1 to 1000 copies of the second compound are loaded into a second alpha granule type of the platelet.

47. The isolated platelet of claim 46, wherein at least one copy of the first compound is loaded into a second alpha granule type of a platelet and at least one copy of the second compound is loaded into a first alpha granule type of the platelet.

48. The isolated platelet of any one of claims 40 to 47, wherein the first GAG-binding peptide preferentially binds to chondroitin sulfate (CS) and the second GAG-binding peptide preferentially binds to heparan sulfate (HS).

49. The isolated platelet of any one of claims 40 to 48, wherein the first GAG-binding peptide preferentially binds to chondroitin sulfate A (CSA) and does not preferably bind to heparan sulfate (HS).

50. The isolated platelet of any one of claims 40 to 49, wherein the first alpha granule type is a P-selectin associated granule and the second alpha granule type von Willebrand factor associated granule.

51. The isolated platelet of any one of claims 40 to 50, wherein contents of the first alpha granule type are released via the high-affinity thrombin receptor PAR1 and contents of the second alpha granule type are released via the low-affinity thrombin receptor PAR4, optionally, the contents of an alpha granule may be released in response to contact with a matrix metalloproteinase (MMP), peroxidase, phosphohydrolase, plasmin, or a plasmin such as tissue plasminogen activator (tPA).

52. The isolated platelet of any one of claims 40 to 51, wherein the contents of the first alpha granule type are released at a lower concentration of thrombin than the concentration of thrombin needed to provide release of the contents of the second alpha granule type.

53. The isolated platelet of any one of claims 40 to 52, wherein the contents first alpha granule type is released before the contents of the second alpha granule type are released.

54. The isolated platelet of any one of claims 40 to 53, wherein the first and the second GAG-binding peptides are each between about 8 amino acids and about 14 amino acids in length.

55. The isolated platelet of claim 54, wherein one or both of the first and the second GAG-binding peptides comprises at least one charged amino acid.

56. The isolated platelet of claim 55, wherein both of the first and the second GAG-binding peptides comprise at least one charged amino acid.

57. The isolated platelet of any one of claims 54 to 56, wherein one or both of the first and the second GAG-binding peptides comprises at least one proline, arginine, and/or isoleucine.

58. The isolated platelet of claim 57, wherein both of the first and the second GAG-binding peptides comprise at least at least one proline, arginine, and/or isoleucine.

59. The isolated platelet of any one of claims 54 to 58, wherein the first and the second GAG-binding peptides independently comprise an amino acid sequence that is at least about 70% identical to one of SEQ ID NO: 1 to SEQ ID NO: 13.

60. The isolated platelet of any one of claims 54 to 59, wherein the first and the second GAG-binding peptides independently comprise an amino acid sequence that is at least about 80% identical to one of SEQ ID NO: 1 to SEQ ID NO: 13.

61. The isolated platelet of any one of claims 54 to 60, wherein the first and the second GAG-binding peptides independently comprise an amino acid sequence that is at least about 90% identical to one of SEQ ID NO: 1 to SEQ ID NO: 13.

62. The isolated platelet of any one of claims 54 to 61, wherein the first and the second GAG-binding peptides independently comprise a charged amino acid at position 1, position 4, position 7, or position 9 with respect to any one of SEQ ID NO: 1 to SEQ ID NO: 13.

63. The isolated platelet of any one of claims 54 to 62, wherein the first and the second GAG-binding peptides independently comprise a proline, arginine, and/or isoleucine at position 1, position 4, position 7, and/or position 9 with respect to any one of SEQ ID NO: 1 to SEQ ID NO: 13.

64. The isolated platelet of any one of claims 54 to 63, wherein the first and the second GAG-binding peptides independently comprise at least 10 amino acids.

65. The isolated platelet of any one of claims 54 to 64, wherein the first and the second GAG-binding peptides independently comprise 11 amino acids.

66. The isolated platelet of any one of claims 54 to 65, wherein the first and the second GAG-binding peptides independently consist of 11 amino acids.

67. The isolated platelet of any one of claims 54 to 66, wherein the GAG-binding peptide consists of the amino acid sequence of one of SEQ ID NO: 1 to SEQ ID NO: 13.

68. The isolated platelet of any one of claims 54 to 66, wherein the first GAG-binding peptide comprises an amino acid sequence that is at least about 90% identical to SEQ ID NO: 1 and the second GAG-binding peptide comprises an amino acid sequence that is at least about 90% identical to SEQ ID NO: 2.

69. The isolated platelet of claim 68, wherein the first GAG-binding peptide comprises the amino acid sequence of SEQ ID NO: 1 and the second GAG-binding peptide comprises the amino acid sequence of SEQ ID NO: 2.

70. The isolated platelet of claim 68 or claim 69, wherein the first GAG-binding peptide consists of the amino acid sequence of SEQ ID NO: 1 and the second GAG-binding peptide consists of the amino acid sequence of SEQ ID NO: 2.

71. The isolated platelet of any one of claims 40 to 70, wherein the first polypeptide consists of the first GAG-binding peptide and the second polypeptide consists of the second GAG-binding peptide.

72. The isolated platelet of any one of claims 40 to 71, wherein the N-terminal of the first polypeptide is directly or indirectly linked to the first agent and/or the N-terminal of the second polypeptide is directly or indirectly linked to second first agent.

73. The isolated platelet of any one of claims 40 to 72, wherein the C-terminal of the first polypeptide is directly or indirectly linked to the first agent and/or the C-terminal of the second polypeptide is directly or indirectly linked to second first agent.

74. The isolated platelet of any one of claims 40 to 73, wherein the first agent is indirectly linked to the first polypeptide via a first linker and/or wherein the second agent is indirectly linked to the second polypeptide via a second linker.

75. The isolated platelet of claim 74, wherein the first linker and/or the second each comprise one or more atoms.

76. The isolated platelet of claim 74 or 75, wherein the first linker and/or the second each comprise a polymer of repeating units.

77. The isolated platelet of any one of claims 74 to 76, wherein the first linker and/or the second each comprise a chain of amino acids.

78. The isolated platelet of any one of claims 40 to 77, wherein the first agent is directly linked to the first polypeptide and/or the second agent is directly linked to the second polypeptide.

79. The isolated platelet of any one of claims 40 to 78, wherein the first agent is directly or indirectly linked to the first polypeptide and/or the second agent is directly or indirectly linked to the second polypeptide using a maleimide reaction, succinimidyl ester reaction, an enzymatic reaction, or another conjugation systems that does not affect protein structure or activity.

80. The isolated platelet of any one of claims 40 to 79, wherein the first agent and/or the second agent independently comprises an antibody, a chemotherapeutic agent, a cytotoxic compound, a small molecule, a fluorescent moiety, radioactive element, an immune checkpoint inhibitor, a growth factor, a growth inhibitor, a protease/proteinase, a coagulation factor, a lipid or phospholipid, an extracellular matrix protein, a hormone, an enzyme, a chemokine/chemoattractant, a neurotrophin, a tyrosine kinase (agonist or inhibitor), or a factor that inhibits cellular proliferation, angiogenesis, inflammation, immunity, or another physiological process mediated by or associated with a platelet.

81. The isolated platelet of claim 80, wherein the first agent and/or the second agent comprises an antibody and/or comprises a fluorescent moiety.

82. The isolated platelet of any one of claims 40 to 81, wherein the first agent and/or the second agent is harmful to mammalian cells and/or is toxic to a subject and/or the first agent and/or the second agent is susceptible to degradation when administered directly into the bloodstream of a subject.

83. The isolated platelet of any one of claims 40 to 82, wherein the first compound and/or the second compound further comprises a fluorescent moiety.

84. The isolated platelet of any one of claims 40 to 83, wherein the first GAG-binding peptide and/or the second GAG-binding peptide also preferentially binds serglycin, perlecan, dermatan sulfate, keratan sulfate, and/or GPIIb/IIIa, optionally, further comprising at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

85. The isolated platelet of any one of claims 40 to 84, wherein, the isolated platelet remains in a resting, fully functional platelet, rather than becoming activated by the loading process.

86. A pharmaceutical composition comprising the isolated platelet of any one of claims 40 to 85 and one or more pharmaceutically acceptable excipients.

87. The pharmaceutical composition of claim 86, further comprising a second isolated platelet comprising at least one copy of a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

88. The pharmaceutical composition of claim 86, further comprising a second isolated platelet comprising at least one copy of the first compound or further comprising a third isolated platelet comprising at least one copy of a second compound.

89. The pharmaceutical composition of claim 88, further comprising a second isolated platelet comprising at least one copy of the first compound and comprising a third isolated platelet comprising at least one copy of a second compound.

90. A use of the pharmaceutical composition of any one of claims 86 to 89 for treating a disease or a disorder.

91. A use of the isolated platelet of any one of claims 40 to 85 or the pharmaceutical composition of any one of claims 86 to 89 in the manufacture of a medicament for treating a disease or disorder.

92. The use of claim 90 or claim 91, wherein the disease or disorder is a cancer.

93. A method for treating a disease or disorder in a subject in need thereof, the method comprising a step of administering to the subject a therapeutically effective amount of the pharmaceutical composition of any one of claims 86 to 89.

94. A method for treating a disease or disorder in a subject in need thereof, the method comprising a step of administering to the subject a therapeutically effective amount of composition of any one of claims 1 to 39.

95. The method of claim 93 or claim 94 wherein the contents of the first alpha granule type is released at a target site before the contents of second alpha granule type is released.

96. The method of any one of claims 93 to 95, further comprising a step of administering to the subject a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase.

97. The method of claim 96, wherein the second pharmaceutical composition promotes release of a first compound from a first alpha granule type and the third pharmaceutical composition promotes release of a second compound from a second alpha granule type.

98. The method of claim 96 or claim 97, wherein the second pharmaceutical composition and/or the third pharmaceutical composition is administered after the pharmaceutical composition is administered.

99. The method of claim 98, wherein the pharmaceutical composition is administered at least twice before the second pharmaceutical composition and/or the third pharmaceutical composition is administered.

100. The method of any one of claims 93 to 99, wherein the disease or disorder is a cancer.

101. The method of any one of claims 93 to 99, wherein the disease of disorder is inflammation.

102. The method of any one of claims 93 to 99, wherein the disease of disorder is a side effect of an implant, graft, stent, or prosthesis.

103. The method of any one of claims 93 to 99, wherein the disease of disorder is caused by a defective gene or the disease or disorder is an injury.

104. The method of any one of claims 93 to 100, wherein the composition comprises an isolated platelet that remains in a resting, fully functional platelet.

105. A method for manufacturing a loaded platelet, the method comprising steps of:
obtaining a platelet,
contacting the platelet in vitro or ex vivo with a composition of any one of claims 1 to 39, and
allowing contact between the platelet and the composition to progress until the first compound is internalized by a first alpha granule type of the platelet and the second compound is internalized by a second alpha granule type of the platelet, thereby producing a loaded platelet.

106. A method for manufacturing a loaded platelet, the method comprising steps of:
obtaining a platelet,
contacting the platelet in vitro or ex vivo with a first compound comprising a first agent and a first polypeptide, wherein the first polypeptide comprises a first glycosaminoglycan (GAG)-binding peptide which is capable of binding a GAG in a first alpha granule type of a platelet; and
contacting the platelet in vitro or ex vivo with a second compound comprising a second agent and a second polypeptide, wherein the second polypeptide comprises a second glycosaminoglycan (GAG)-binding peptide which is capable of binding a GAG in a second alpha granule type of the platelet.

107. The method of claim 106, wherein the contacting the platelet with the first compound and the contacting the platelet with the second compound are contemporaneous.

108. The method of claim 106, wherein the contacting the platelet with the first compound and the contacting the platelet with the second compound are sequential.

109. The method of any one of claims 106 to 108, further comprising contacting the platelet in vitro or ex vivo with a third compound comprising a third agent and a third polypeptide, wherein the third polypeptide comprises a third glycosaminoglycan (GAG)-binding peptide which is capable of binding a GAG in a third alpha granule type of a platelet; and wherein the third GAG-binding peptide preferentially binds serglycin, perlecan, dermatan sulfate, keratan sulfate, and/or GPIIb/IIIa.

110. The method of any one of claims 106 to 109, wherein contacting the platelet with the first compound and or the second compound does not activate the platelet and, instead, the platelet remains as a resting, fully functional platelet.

111. A kit for treating a disease or disorder comprising the isolated platelet of any one of claims 40 to 85 and instructions for use.

112. A kit for treating a disease or disorder comprising the pharmaceutical composition of any one of claims 86 to 89 and instructions for use.

113. The kit of claim 111 or claim 112 further comprising a second pharmaceutical composition and/or a third pharmaceutical composition, independently, comprising one or more of heparanase, thrombin and its fragment peptides, a protease-activated receptor 1 (PAR1) agonist or antagonist peptide, a protease-activated receptor 4 (PAR4) agonist or antagonist peptide, plasmin and its fragments, a metalloproteinase, a peroxidase, and/or a phosphohydrolase.

114. A kit for manufacturing a loaded platelet comprising a composition of any one of claims 1 to 39 and instructions for use.


SEQUENCE LISTING (14 sequences)

SEQ ID NO: 1 - protein, 11 aa, synthetic construct
  REGION 1..11: note = Synthetic Peptide
ERRIWFPYRRF

SEQ ID NO: 2 - protein, 11 aa, synthetic construct
  REGION 1..11: note = Synthetic Peptide
RFRWPYRIREF

SEQ ID NO: 3 - protein, 11 aa, synthetic construct
  REGION 1..11: note = Synthetic Peptide
ARRIWFPYRRF

SEQ ID NO: 4 - protein, 11 aa, synthetic construct
  REGION 1..11: note = Synthetic Peptide
EARIWFPYRRF

SEQ ID NO: 5 - protein, 11 aa, synthetic construct
  REGION 1..11: note = Synthetic Peptide
ERAIWFPYRRF

SEQ ID NO: 6 - protein, 11 aa, synthetic construct
  REGION 1..11: note = Synthetic Peptide
ERRAWFPYRRF

SEQ ID NO: 7 - protein, 11 aa, synthetic construct
  REGION 1..11: note = Synthetic Peptide
ERRIAFPYRRF

SEQ ID NO: 8 - protein, 11 aa, synthetic construct
  REGION 1..11: note = Synthetic Peptide
ERRIWAPYRRF

SEQ ID NO: 9 - protein, 11 aa, synthetic construct
  REGION 1..11: note = Synthetic Peptide
ERRIWFAYRRF

SEQ ID NO: 10 - protein, 11 aa, synthetic construct
  REGION 1..11: note = Synthetic Peptide
ERRIWFPARRF

SEQ ID NO: 11 - protein, 11 aa, synthetic construct
  REGION 1..11: note = Synthetic Peptide
ERRIWFPYARF

SEQ ID NO: 12 - protein, 11 aa, synthetic construct
  REGION 1..11: note = Synthetic Peptide
ERRIWFPYRAF

SEQ ID NO: 13 - protein, 11 aa, synthetic construct
  REGION 1..11: note = Synthetic Peptide
ERRIWFPYRRA

SEQ ID NO: 14 - protein, 11 aa, synthetic construct
  REGION 1..11: note = Synthetic Peptide
EGGIWFPYGGF